SEQUENCE LISTING (42 sequences)

SEQ ID NO: 1 - protein, 10 aa, synthetic construct
  REGION 1..10: note = Synthetic Construct
GFTFSDSWIH

SEQ ID NO: 2 - protein, 18 aa, synthetic construct
  REGION 1..18: note = Synthetic Construct
AWISPYGGSTYYADSVKG

SEQ ID NO: 3 - protein, 9 aa, synthetic construct
  REGION 1..9: note = Synthetic Construct
RHWPGGFDY

SEQ ID NO: 4 - protein, 11 aa, synthetic construct
  REGION 1..11: note = Synthetic Construct
RASQDVSTAVA

SEQ ID NO: 5 - protein, 7 aa, synthetic construct
  REGION 1..7: note = Synthetic Construct
SASFLYS

SEQ ID NO: 6 - protein, 9 aa, synthetic construct
  REGION 1..9: note = Synthetic Construct
QQYLYHPAT

SEQ ID NO: 7 - protein, 118 aa, synthetic construct [sequence omitted]

SEQ ID NO: 8 - protein, 108 aa, synthetic construct
  REGION 1..108: note = Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 9 - protein, 447 aa, synthetic construct
  REGION 1..447: note = Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 214 aa, synthetic construct
  REGION 1..214: note = Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 439 aa, synthetic construct
  REGION 1..439: note = Synthetic Construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 12 - protein, 214 aa, synthetic construct
  REGION 1..214: note = Synthetic Construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 446 aa, synthetic construct
  REGION 1..446: note = Synthetic Construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 14 - protein, 218 aa, synthetic construct
  REGION 1..218: note = Synthetic Construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 449 aa, synthetic construct
  REGION 1..449: note = Synthetic Construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 216 aa, synthetic construct
  REGION 1..216: note = Synthetic Construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 17 - protein, 450 aa, synthetic construct
  REGION 1..450: note = Synthetic Construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 215 aa, synthetic construct
  REGION 1..215: note = Synthetic Construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - RNA, 129 nt, synthetic construct
  misc_feature 1..129: note = Synthetic Construct
  source 1..129: mol_type = other RNA
ggcgaactagtattcttctggtccccacagactcagagagaacccgccaccatgagagtgatggcccccagaaccctgatcctgctgctgtctggcgccctggccctgacagagacatgggccggaagc

SEQ ID NO: 20 - RNA, 488 nt, synthetic construct
  misc_feature 1..488: note = Synthetic Construct
  source 1..488: mol_type = other RNA
atcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctagtaactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgct

SEQ ID NO: 21 - RNA, 51 nt, synthetic construct
  misc_feature 1..51: note = Synthetic Construct
  source 1..51: mol_type = other RNA
ggcgaactagtattcttctggtccccacagactcagagagaacccgccacc

SEQ ID NO: 22 - DNA, 51 bp, synthetic construct
  misc_feature 1..51: note = Synthetic Construct
  source 1..51: mol_type = other DNA
ggcgaactagtattcttctggtccccacagactcagagagaacccgccacc

SEQ ID NO: 23 - RNA, 39 nt, synthetic construct
  misc_feature 1..39: note = Synthetic Construct
  source 1..39: mol_type = other RNA
ttcttctggtccccacagactcagagagaacccgccacc

SEQ ID NO: 24 - DNA, 39 bp, synthetic construct
  misc_feature 1..39: note = Synthetic Construct
  source 1..39: mol_type = other DNA
ttcttctggtccccacagactcagagagaacccgccacc

SEQ ID NO: 25 - RNA, 78 nt, synthetic construct
  misc_feature 1..78: note = Synthetic Construct
  source 1..78: mol_type = other RNA
atgagagtgatggcccccagaaccctgatcctgctgctgtctggcgccctggccctgacagagacatgggccggaagc

SEQ ID NO: 26 - DNA, 78 bp, synthetic construct
  misc_feature 1..78: note = Synthetic Construct
  source 1..78: mol_type = other DNA
atgagagtgatggcccccagaaccctgatcctgctgctgtctggcgccctggccctgacagagacatgggccggaagc

SEQ ID NO: 27 - protein, 26 aa, synthetic construct
  REGION 1..26: note = Synthetic Construct
MRVMAPRTLILLLSGALALTETWAGS

SEQ ID NO: 28 - RNA, 165 nt, synthetic construct
  misc_feature 1..165: note = Synthetic Construct
  source 1..165: mol_type = other RNA
atcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcc

SEQ ID NO: 29 - DNA, 165 bp, synthetic construct
  misc_feature 1..165: note = Synthetic Construct
  source 1..165: mol_type = other DNA
atcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcc

SEQ ID NO: 30 - protein, 55 aa, synthetic construct
  REGION 1..55: note = Synthetic Construct
IVGIVAGLAVLAVVVIGAVVATVMCRRKSSGGKGGSYSQAASSDSAQGSDVSLTA

SEQ ID NO: 31 - RNA, 317 nt, synthetic construct
  misc_feature 1..317: note = Synthetic Construct
  source 1..317: mol_type = other RNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgct

SEQ ID NO: 32 - DNA, 311 bp, synthetic construct
  misc_feature 1..311: note = Synthetic Construct
  source 1..311: mol_type = other DNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgct

SEQ ID NO: 33 - RNA, 136 nt, synthetic construct
  misc_feature 1..136: note = Synthetic Construct
  source 1..136: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcc

SEQ ID NO: 34 - DNA, 136 bp, synthetic construct
  misc_feature 1..136: note = Synthetic Construct
  source 1..136: mol_type = other DNA
ctggtactgcatgcacgcaatgctagctgcccctttcccgtcctgggtaccccgagtctcccccgacctcgggtcccaggtatgctcccacctccacctgccccactcaccacctctgctagttccagacacctcc

SEQ ID NO: 35 - RNA, 143 nt, synthetic construct
  misc_feature 1..143: note = Synthetic Construct
  source 1..143: mol_type = other RNA
caagcacgcagcaatgcagctcaaaacgcttagcctagccacacccccacgggaaacagcagtgattaacctttagcaataaacgaaagtttaactaagctatactaaccccagggttggtcaatttcgtgccagccacaccg

SEQ ID NO: 36 - DNA, 143 bp, synthetic construct
  misc_feature 1..143: note = Synthetic Construct
  source 1..143: mol_type = other DNA
caagcacgcagcaatgcagctcaaaacgcttagcctagccacacccccacgggaaacagcagtgattaacctttagcaataaacgaaagtttaactaagctatactaaccccagggttggtcaatttcgtgccagccacaccg

SEQ ID NO: 37 - RNA, 30 nt, synthetic construct
  misc_feature 1..30: note = Synthetic Construct
  source 1..30: mol_type = other RNA
ggcggctctggaggaggcggctccggaggc

SEQ ID NO: 38 - DNA, 30 bp, synthetic construct
  misc_feature 1..30: note = Synthetic Construct
  source 1..30: mol_type = other DNA
ggcggctctggaggaggcggctccggaggc

SEQ ID NO: 39 - protein, 10 aa, synthetic construct
  REGION 1..10: note = Synthetic Construct
GGSGGGGSGG

SEQ ID NO: 40 - DNA, 129 bp, synthetic construct
  misc_feature 1..129: note = Synthetic Construct
  source 1..129: mol_type = other DNA
ggcgaactagtattcttctggtccccacagactcagagagaacccgccaccatgagagtgatggcccccagaaccctgatcctgctgctgtctggcgccctggccctgacagagacatgggccggaagc

SEQ ID NO: 41 - DNA, 488 bp, synthetic construct
  misc_feature 1..488: note = Synthetic Construct
  source 1..488: mol_type = other DNA
atcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctagtaactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgct

SEQ ID NO: 42 - RNA, 740 nt, synthetic construct
  misc_feature 1..740: note = Synthetic Construct
  misc_feature 1..2: note = Linkage between the first two G residues is the unusual bond (5'->5')-pp(s)p-, e.g., as shown in Table 1 and in FIG. 3 of the specification
  misc_difference 132: note = n = A,T,C, G or U
  misc_difference 132: note = Exists as polynucleotide sequence(s) as defined in the specification and may encode neoepitopes, optionally separated by linkers, as defined in the specification (e.g., FIGS. 1-2)
  source 1..740: mol_type = other RNA
ggggcgaactagtattcttctggtccccacagactcagagagaacccgccaccatgagagtgatggcccccagaaccctgatcctgctgctgtctggcgccctggccctgacagagacatgggccggaagcnatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctagtaactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2025/0367275 A1
(43) Pub. Date: Dec. 4, 2025

(54) METHODS OF TREATING PANCREATIC CANCER WITH A PD-1 AXIS BINDING ANTAGONIST AND AN RNA VACCINE

(71) Applicants: Genentech, Inc., South San Francisco, CA (US); BioNTech SE, Mainz (DE)

(72) Inventors: Michael Robert MANCUSO, San Carlos, CA (US); Jill M. SCHARTNER, Brisbane, CA (US); Alireza TAFAZZOL, San Francisco, CA (US); Mahesh YADAV, San Bruno, CA (US); Leesun Hannah KIM, Burlingame, CA (US); Ugur SAHIN, Mainz (DE); Özlem TÜRECI, Mainz (DE)

(73) Assignees: Genentech, Inc., South San Francisco, CA (US); BioNTech SE, Mainz (DE)

(21) Appl. No.: 19/242,465

(22) Filed: Jun. 18, 2025

Related U.S. Application Data
(63) Continuation of application No. PCT/US2023/084762, filed on Dec. 19, 2023.
(60) Provisional application No. 63/476,246, filed on Dec. 20, 2022.
(60) Provisional application No. 63/508,248, filed on Jun. 14, 2023.

(51) Int. Cl. A61K 39/00; A61K 9/00; A61K 9/1272; A61K 9/51; A61K 31/337; A61K 31/475; A61K 31/513; A61K 31/519; A61K 31/555; A61K 33/243; A61P 35/00; C07K 14/74; C07K 16/28; C12Q 1/6886

(52) U.S. Cl. CPC A61K 39/001111; A61K 9/0019; A61K 9/1272; A61K 9/5123; A61K 31/337; A61K 31/475; A61K 31/513; A61K 31/519; A61K 31/555; A61K 33/243; A61P 35/00; C07K 14/70539; C07K 16/2818; C07K 16/2827; C12Q 1/6886; A61K 2039/505; A61K 2039/53; A61K 2039/54; A61K 2039/545; A61K 2039/55555; C07K 2317/565; C07K 2319/02; C12Q 2600/106

(57) ABSTRACT

The present disclosure provides methods for treating an individual with pancreatic cancer with an individualized cancer vaccine and a PD-1 axis antagonist.

### FIELD

The present disclosure relates to methods for treating an individual with pancreatic cancer with an individualized cancer vaccine and a PD-1 axis antagonist.

### CROSS-REFERENCE TO RELATED APPLICATIONS

This application claims the benefit of U.S. Provisional Patent Application No. 63/508,248, filed on Jun. 14, 2023, and U.S. Provisional Patent Application No. 63/476,246, filed on Dec. 20, 2022, the entire contents of each of which are incorporated herein by reference.

### REFERENCE TO AN ELECTRONIC SEQUENCE LISTING

The contents of the electronic sequence listing (146392064940seqlist.xml; Size: 62,168 bytes; and Date of Creation: Dec. 7, 2023) are herein incorporated by reference in their entirety.

### BACKGROUND

Pancreatic cancer is the seventh leading cause of cancer deaths worldwide, and the third leading cause of cancer deaths in the United States and Europe (Dalmartello et al., Ann Oncol 2022; 33:330-9; and Siegel et al., CA Cancer J Clin 2022; 72:7-33). Pancreatic ductal adenocarcinoma (PDAC), which develops in the exocrine tissue of the pancreas, is responsible for approximately 90% of pancreatic cancer cases. PDAC has a 5-year survival rate under 10% (Haeberle and Esposito. Transl Gastroenterol Hepatol 2019; 4:50). Currently, the only potentially curative treatment for PDAC is surgical resection (Rawla et al., World J Oncol 2019; 10:10⁻²⁷; Park et al., JAMA 2021; 326:851-62). However, the 5-year survival rate for patients with PDAC that do undergo resection is reported to be as low as 12%, depending on the patient population (Bilimoria et al., Cancer 2007; 110:1227-34; Ferrone et al., J Gastrointest Surg 2008; 12:701-6; Katz et al., Ann Surg Oncol 2009; 16:836-47; Ferrone et al., Surgery 2012; 152 (3 Suppl 1): S43-9; He et al., HPB (Oxford) 2014; 16:83-90; and Conroy et al., JAMA Oncol 2022;e223829. doi: 10.1001/jamaoncol.20223820).

Immunotherapies, such as immune checkpoint inhibitors, provide clinical benefit for patients with multiple types of solid tumors, including patients with mismatch repair deficient/microsatellite instability-high PDAC (Le et al., Science 2017; 357:409-13; and Marabelle et al., J Clin Oncol 2020; 38:1-10). However, the majority (>98%) of patients with PDAC have mismatch repair proficient/microsatellite-stable disease and do not respond to immune checkpoint inhibition (O'Reilly et al., JAMA Oncol 2019; 5:1431-8; and Bian and Almhanna. Transl Gastroenterol Hepatol 2021; 6:6). The poor immunogenicity of PDAC has been attributed to its immunosuppressive tumor microenvironment, paucity of tumor-infiltrating lymphocytes, and low tumor mutational burden, which lead to expression of a limited number of immunogenic neoantigens (Lutz et al., Cancer Immunol Res 2014; 2:616-31; and Schizas et al., Cancer Treat Rev 2020; 86:102016).

Therapeutic vaccines targeting immunogenic epitopes to activate the immune system against cancer are being developed and investigated, and may be beneficial for the treatment of cancers that have poor immunogenicity, such as pancreatic cancers, including PDAC. However, thus far, therapeutic vaccines, while promising, have historically fallen short of expectations. One of the potential reasons is that cancer-specific T cells become functionally exhausted during chronic exposure to cancer cells. Thus, combination therapy regimens employing two or more targeted cancer immunotherapy agents, e.g., an immune checkpoint inhibitor, a therapeutic vaccine targeting immunogenic epitopes, and chemotherapy, may be required to fully engage the anti-tumor potential of the host immune system. For example, a recent phase I study of an individualized RNA vaccine combined with atezolizumab and a chemotherapy regimen in pancreatic ductal adenocarcinoma showed an acceptable safety profile and promising RNA vaccine-induced immune responses (see, e.g., Balachandran et al., Journal of Clinical Oncology 40, no. 16_suppl (Jun. 1, 2022) 2516-2516). However, a need remains for improved methods for treating pancreatic cancers, such as PDAC.

All references cited herein, including patent applications, patent publications, and UniProtKB/Swiss-Prot Accession numbers are herein incorporated by reference in their entirety, as if each individual reference were specifically and individually indicated to be incorporated by reference.

### SUMMARY

Provided herein is a method for treating a pancreatic cancer tumor in a human patient in need thereof, comprising administering to the patient: (a) an individualized RNA vaccine comprising one or more polynucleotides encoding one or more neoepitopes resulting from cancer-specific somatic mutations present in a pancreatic cancer tumor specimen obtained from the patient, (b) a PD-1 axis binding antagonist, and (c) a chemotherapeutic treatment; wherein the RNA vaccine, the PD-1 axis binding antagonist, and the chemotherapeutic treatment are administered to the patient during a priming phase, a chemotherapy phase after the priming phase, and a boost phase after the chemotherapy phase, wherein: (i) the priming phase comprises administering to the patient at least one dose of the RNA vaccine and at least one dose of the PD-1 axis binding antagonist, (ii) the chemotherapy phase comprises administering to the patient the chemotherapeutic treatment, and (iii) the boost phase comprises administering to the patient at least one dose of the RNA vaccine and at least one dose of the PD-1 axis binding antagonist.

In some embodiments, the pancreatic cancer tumor is a pancreatic ductal adenocarcinoma (PDAC) tumor. In some embodiments, the pancreatic cancer tumor is resectable.

In some embodiments, the priming phase begins at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 weeks, at least about 14 weeks, or at least about 15 weeks after resection of the pancreatic cancer tumor from the patient. In some embodiments, the priming phase begins between about 6 weeks and about 12 weeks after resection of the pancreatic cancer tumor from the patient.

In some embodiments, the priming phase comprises administering one dose of the PD-1 axis binding antagonist. In some embodiments, the priming phase comprises administering the PD-1 axis binding antagonist on day 1 of week 3 of the priming phase.

In some embodiments, the priming phase comprises administering at least two doses of the PD-1 axis binding antagonist. In some embodiments, the priming phase comprises administering the PD-1 axis binding antagonist once every four weeks. In some embodiments, the priming phase comprises administering the PD-1 axis binding antagonist on day 1 of week 1 of the priming phase and every four weeks thereafter. In some embodiments, the priming phase comprises administering two doses of the PD-1 axis binding antagonist. In some embodiments, the priming phase comprises administering the PD-1 axis binding antagonist on day 1 of week 1 and on day 1 of week 5 of the priming phase.

In some embodiments, the priming phase comprises administering any of 2, 3, 4, 5, 6, 7, or 8 doses of the RNA vaccine. In some embodiments, the priming phase comprises administering 2 or 3 doses of the RNA vaccine. In some embodiments, priming phase comprises administering between 6 and 8 doses of the RNA vaccine, or up to six doses of the RNA vaccine. In some embodiments, the priming phase comprises administering 6 doses of the RNA vaccine. In some embodiments, the priming phase comprises administering the RNA vaccine once per week. In some embodiments, the priming phase comprises administering the RNA vaccine on day 1 of week 1 of the priming phase and once per week thereafter. In some embodiments, the priming phase comprises administering six doses of the RNA vaccine. In some embodiments, the priming phase comprises administering the RNA vaccine on day 1 of weeks 1, 2, 3, 4, 5, and 6 of the priming phase.

In some embodiments, each dose of the PD-1 axis binding antagonist administered to the patient during the priming phase is administered on the same day as administration of a dose of the RNA vaccine. In some embodiments, the priming phase comprises six weeks. In some embodiments, the RNA vaccine is administered on day 1 of weeks 1, 2, 3, 4, 5, and 6 of the priming phase, and the PD-1 axis binding antagonist is administered on day 1 of week 3 of the priming phase. In some embodiments, the RNA vaccine is administered on day 1 of weeks 1, 2, 3, 4, 5, and 6 of the priming phase, and the PD-1 axis binding antagonist is administered on day 1 of weeks 1 and 5 of the priming phase.

In some embodiments, the chemotherapy phase comprises administering the chemotherapeutic treatment for at least about 8 weeks, at least about 9 weeks, at least about 10 weeks, at least about 11 weeks, at least about 12 weeks, at least about 13 weeks, at least about 14 weeks, at least about 15 weeks, at least about 16 weeks, at least about 17 weeks, at least about 18 weeks, at least about 19 weeks, at least about 20 weeks, at least about 21 weeks, at least about 22 weeks, at least about 23 weeks, at least about 24 weeks, at least about 25 weeks, at least about 26 weeks, at least about 27 weeks, at least about 28 weeks, at least about 29 weeks, at least about 30 weeks, or more. In some embodiments, the chemotherapy phase comprises administering the chemotherapeutic treatment for 23 weeks. In some embodiments, the chemotherapeutic treatment is administered once every two weeks. In some embodiments, the chemotherapy phase comprises administering the chemotherapeutic treatment on day 1 of week 1 of the chemotherapy phase and every two weeks thereafter. In some embodiments, the chemotherapy phase comprises administering at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, or at least 24, or more, administrations of the chemotherapeutic treatment. In some embodiments, the chemotherapy phase comprises administering 12 administrations of the chemotherapeutic treatment. In some embodiments, the chemotherapy phase comprises 24 weeks. In some embodiments, the chemotherapy phase comprises administering the chemotherapeutic treatment on day 1 of weeks 1, 3, 5, 7, 9, 11, 13, 15, 17, 19, 21, and 23 of the chemotherapy phase.

In some embodiments, the chemotherapy phase begins at least about 1 week, at least about 2 weeks, at least about 3 weeks, or at least about 4 weeks after the end of the priming phase and after the last administration of the RNA vaccine. In some embodiments, the chemotherapy phase begins no later than week 9, timing starting with week 1 of the priming phase. In some embodiments, the priming phase comprises six weeks, and wherein the chemotherapy phase begins no later than week 9, timing starting with week 1 of the priming phase. In some embodiments, the priming phase comprises six weeks, and wherein the chemotherapy phase comprises administering the chemotherapeutic treatment starting on day 1 of week 7 and every two weeks thereafter, timing starting with week 1 of the priming phase.

In some embodiments, the chemotherapy phase comprises administering 12 administrations of the chemotherapeutic treatment. In some embodiments, the chemotherapy phase comprises administering the chemotherapeutic treatment on day 1 of weeks 7, 9, 11, 13, 15, 17, 19, 21, 23, 25, 27, and 29, timing starting with week 1 of the priming phase.

In some embodiments, the boost phase comprises administering 2, 3, 4, 5, 6, 7, or 8 doses of the RNA vaccine. In some embodiments, the boost phase comprises administering 2, 3, 4, 5, 6, 7, or 8 doses of the PD-1 axis binding antagonist. In some embodiments, the boost phase comprises administering 6 doses of the PD-1 axis binding antagonist and 6 doses of the RNA vaccine. In some embodiments, the boost phase comprises administering the PD-1 axis binding antagonist and the RNA vaccine once every four weeks. In some embodiments, the boost phase comprises administering the PD-1 axis binding antagonist on day 1 of week 1 of the boost phase and every four weeks thereafter. In some embodiments, the boost phase comprises administering the RNA vaccine on day 1 of week 1 of the boost phase and every four weeks thereafter. In some embodiments, administrations of the RNA vaccine and the PD-1 axis binding antagonist during the boost phase occur on the same day. In some embodiments, the boost phase comprises administering the PD-1 axis binding antagonist and the RNA vaccine on day 1 of week 1 of the boost phase and every four weeks thereafter. In some embodiments, the boost phase comprises 21 weeks. In some embodiments, the RNA vaccine and the PD-1 axis binding antagonist are administered on day 1 of weeks 1, 5, 9, 13, 17, and 21 of the boost phase.

In some embodiments, the boost phase begins at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 weeks, at least about 14 weeks, or at least about 15 weeks after the end of the chemotherapy phase. In some embodiments, the boost phase begins up to about 12 weeks after the end of the chemotherapy phase, optionally up to about 12 weeks after the last administration of the chemotherapeutic treatment. In some embodiments, the boost phase begins: between about 3 weeks to about 12 weeks after the end of the chemotherapy phase, optionally between about 3 weeks to about 12 weeks after the last administration of the chemotherapeutic treatment; or about three weeks or about four weeks after the end of the chemotherapy phase, optionally about three weeks or about four weeks after the last administration of the chemotherapeutic treatment.

In some embodiments, the boost phase begins on week 27, timing starting with week 1 of the chemotherapy phase. In some embodiments, the boost phase begins on week 33, timing starting with week 1 of the priming phase. In some embodiments, the boost phase comprises administering the RNA vaccine and the PD-1 axis binding antagonist on day 1 of week 33 and every four weeks thereafter, timing starting with week 1 of the priming phase.

In some embodiments, the RNA vaccine and the PD-1 axis binding antagonist are administered for six administrations during the boost phase. In some embodiments, the boost phase comprises administering the RNA vaccine and the PD-1 axis binding antagonist on day 1 of weeks 33, 37, 41, 45, 49, and 53, timing starting with week 1 of the priming phase.

In some embodiments, (a) the priming phase comprises administering the RNA vaccine on day 1 of weeks 1, 2, 3, 4, 5, and 6 of the priming phase, and the PD-1 axis binding antagonist on day 1 of week 3 of the priming phase; (b) the chemotherapy phase comprises administering the chemotherapeutic treatment on day 1 of weeks 7, 9, 11, 13, 15, 17, 19, 21, 23, 25, 27, and 29, timing starting with week 1 of the priming phase; and (c) the boost phase comprises administering the RNA vaccine and the PD-1 axis binding antagonist on day 1 of weeks 33, 37, 41, 45, 49, and 53, timing starting with week 1 of the priming phase.

In some embodiments, (a) the priming phase comprises administering the RNA vaccine on day 1 of weeks 1, 2, 3, 4, 5, and 6 of the priming phase, and the PD-1 axis binding antagonist on day 1 of weeks 1 and 5 of the priming phase; (b) the chemotherapy phase comprises administering the chemotherapeutic treatment on day 1 of weeks 7, 9, 11, 13, 15, 17, 19, 21, 23, 25, 27, and 29, timing starting with week 1 of the priming phase; and (c) the boost phase comprises administering the RNA vaccine and the PD-1 axis binding antagonist on day 1 of weeks 33, 37, 41, 45, 49, and 53, timing starting with week 1 of the priming phase.

In some embodiments, the priming phase begins between about 6 weeks and about 12 weeks after resection of the pancreatic cancer tumor from the patient.

In some embodiments, the PD-1 axis binding antagonist is a PD-1 binding antagonist. In some embodiments, the PD-1 binding antagonist is an anti-PD-1 antibody. In some embodiments, the anti-PD-1 antibody is nivolumab or pembrolizumab. In some embodiments, the PD-1 axis binding antagonist is a PD-L1 binding antagonist. In some embodiments, the PD-L1 binding antagonist is an anti-PD-L1 antibody. In some embodiments, the anti-PD-L1 antibody is avelumab or durvalumab. In some embodiments, the anti-PD-L1 antibody comprises: (a) a heavy chain variable region (VH) that comprises an HVR-H1 comprising an amino acid sequence GFTFSDSWIH (SEQ ID NO:1), an HVR-H2 comprising an amino acid sequence AWISPYGGSTYYADSVKG (SEQ ID NO:2), and HVR-H3 comprising an amino acid sequence RHWPGGFDY (SEQ ID NO:3), and (b) a light chain variable region (VL) that comprises an HVR-L1 comprising an amino acid sequence RASQDVSTAVA (SEQ ID NO: 4), an HVR-L2 comprising an amino acid sequence SASFLYS (SEQ ID NO:5), and an HVR-L3 comprising an amino acid sequence QQYLYHPAT (SEQ ID NO:6). In some embodiments, the anti-PD-L1 antibody comprises a heavy chain variable region (VH) comprising an amino acid sequence of SEQ ID NO:7 and a light chain variable region (VL) comprising an amino acid sequence of SEQ ID NO:8. In some embodiments, the anti-PD-L1 antibody is atezolizumab.

In some embodiments, the PD-1 axis binding antagonist is administered intravenously to the patient. In some embodiments, the anti-PD-L1 antibody is administered to the patient at a dose of about 1200 mg or about 1680 mg. In some embodiments, the anti-PD-L1 antibody is atezolizumab, and the atezolizumab is administered intravenously to the patient at a dose of about 1680 mg.

In some embodiments, the chemotherapeutic treatment comprises one or more of gemcitabine, leucovorin, 5-fluorouracil, capecitabine, irinotecan, liposomal irinotecan, a platinum-based chemotherapeutic agent, a taxane, and any combination thereof. In some embodiments, the platinum-based chemotherapeutic agent is cisplatin, oxaliplatin, or both. In some embodiments, the taxane is paclitaxel, docetaxel, albumin-bound paclitaxel, or any combination thereof. In some embodiments, the chemotherapeutic treatment comprises leucovorin, 5-fluorouracil, irinotecan, and oxaliplatin. In some embodiments, the chemotherapeutic treatment is a FOLFIRINOX treatment or an mFOLFIRINOX treatment.

In some embodiments, the chemotherapeutic treatment comprises: oxaliplatin at a dose of about 85 mg/m2; leucovorin at a dose of about 400 mg/m2; irinotecan at a dose of about 150 mg/m2; and/or 5-fluorouracil at a dose of about 2400 mg/m2. In some embodiments, the chemotherapeutic treatment is administered intravenously to the patient.

In some embodiments, the RNA vaccine comprises one or more polynucleotides encoding 5-20 or 10⁻²⁰ neoepitopes resulting from cancer-specific somatic mutations present in the tumor specimen. In some embodiments, the one or more polynucleotides of the RNA vaccine are formulated with one or more lipids. In some embodiments, the one or more polynucleotides of the RNA vaccine and the one or more lipids form a lipid nanoparticle. In some embodiments, the one or more polynucleotides of the RNA vaccine and the one or more lipids form a lipoplex. In some embodiments, the lipid nanoparticle or lipoplex comprises one or more lipids that form a multilamellar structure that encapsulates the one or more polynucleotides of the RNA vaccine.

In some embodiments, the one or more lipids comprise at least one cationic lipid and at least one helper lipid. In some embodiments, the one or more lipids comprise (R) N,N,N-trimethyl-2,3-dioleyloxy-1-propanaminium chloride (DOTMA) and 1,2-dioleoyl-sn-glycero-3-phosphoethanolamine (DOPE). In some embodiments, at physiological pH the overall charge ratio of positive charges to negative charges of the lipid nanoparticle or lipoplex is 1.3:2 (0.65).

In some embodiments, the one or more polynucleotides of the RNA vaccine are RNA molecules, optionally messenger RNA molecules. In some embodiments, the RNA vaccine is administered to the patient at a dose of about 15 μg, about 21 μg, about 21.3 μg, about 25 μg, about 38 μg, or about 50 μg. In some embodiments, the RNA vaccine is administered to the patient at a dose of about 25 μg. In some embodiments, the RNA vaccine dose is administered to the patient in two equal half-doses. In some embodiments, the two equal half-doses are administered sequentially, optionally with an observation period between the administered equal half-doses. In some embodiments, the dose of about 25 μg is split into two equal half-doses of about 12.5 μg, each administered over 1 minute, optionally with a 5-minute observation period between the administered equal half-doses. In some embodiments, the RNA vaccine is administered intravenously to the patient.

In some embodiments, the RNA vaccine comprises an RNA molecule comprising, in the 5′→3′ direction: (1) a 5′ cap; (2) a 5′ untranslated region (UTR); (3) a polynucleotide sequence encoding a secretory signal peptide; (4) a polynucleotide sequence encoding the one or more neoepitopes resulting from cancer-specific somatic mutations present in the tumor specimen; (5) a polynucleotide sequence encoding at least a portion of a transmembrane and cytoplasmic domain of a major histocompatibility complex (MHC) molecule; (6) a 3′ UTR comprising: (a) a 3′ untranslated region of an Amino-Terminal Enhancer of Split (AES) mRNA or a fragment thereof; and (b) non-coding RNA of a mitochondrially encoded 12S RNA or a fragment thereof; and (7) a poly(A) sequence.

In some embodiments, the RNA molecule further comprises a polynucleotide sequence encoding an amino acid linker; wherein the polynucleotide sequences encoding the amino acid linker and a first of the one or more neoepitopes form a first linker-neoepitope module; and wherein the polynucleotide sequences forming the first linker-neoepitope module are between the polynucleotide sequence encoding the secretory signal peptide and the polynucleotide sequence encoding the at least portion of the transmembrane and cytoplasmic domain of the MHC molecule in the 5′→3′ direction.

In some embodiments, the amino acid linker comprises the sequence GGSGGGGSGG (SEQ ID NO:39). In some embodiments, the polynucleotide sequence encoding the amino acid linker comprises the sequence GGCGGCUCUGGAGGAGGCGGCUCCGGAGGC (SEQ ID NO:37).

In some embodiments, the RNA molecule further comprises, in the 5′→3′ direction: at least a second linker-neoepitope module, wherein the at least second linker-neoepitope module comprises a polynucleotide sequence encoding an amino acid linker and a polynucleotide sequence encoding a neoepitope; wherein the polynucleotide sequences forming the second linker-neoepitope module are between the polynucleotide sequence encoding the neoepitope of the first linker-neoepitope module and the polynucleotide sequence encoding the at least portion of the transmembrane and cytoplasmic domain of the MHC molecule in the 5′→3′ direction; and wherein the neoepitope of the first linker-neoepitope module is different from the neoepitope of the second linker-neoepitope module.

In some embodiments, the RNA molecule comprises 5 linker-neoepitope modules, and wherein the 5 linker-neoepitope modules each encode a different neoepitope. In some embodiments, the RNA molecule comprises 10 linker-neoepitope modules, and wherein the 10 linker-neoepitope modules each encode a different neoepitope. In some embodiments, the RNA molecule comprises 20 linker-neoepitope modules, and wherein the 20 linker-neoepitope modules each encode a different neoepitope.

In some embodiments, the RNA molecule further comprises a second polynucleotide sequence encoding an amino acid linker, wherein the second polynucleotide sequence encoding the amino acid linker is between the polynucleotide sequence encoding the neoepitope that is most distal in the 3′ direction and the polynucleotide sequence encoding the at least portion of the transmembrane and cytoplasmic domain of the MHC molecule.

In some embodiments, the 5′ cap comprises a DI diastereoisomer of the structure:

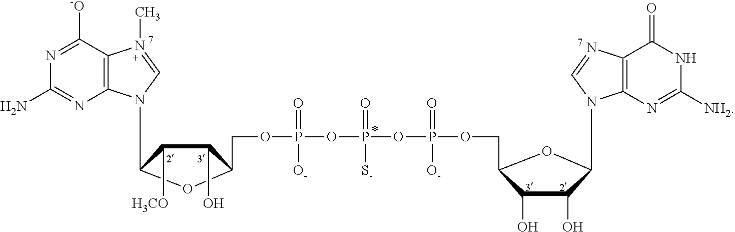

In some embodiments, the 5′ UTR comprises the sequence UUCUUCUGGUCCCCACAGACUCAGAGAGAACCCGCCACC (SEQ ID NO:23). In some embodiments, the 5′ UTR comprises the sequence

(SEQ ID NO: 21)
GGCGAACUAGUAUUCUUCUGGUCCCCACAGACUCAGAGAGAACCC
GCCACC.

In some embodiments, the secretory signal peptide comprises the amino acid sequence MRVMAPRTLILLLSGALALTETWAGS (SEQ ID NO:27). In some embodiments, the polynucleotide sequence encoding the secretory signal peptide comprises the sequence

(SEQ ID NO: 25)
AUGAGAGUGAUGGCCCCCAGAACCCUGAUCCUGCUGCUGUCUGGC
GCCCUGGCCCUGACAGAGACAUGGGCCGGAAGC.

In some embodiments, the at least portion of the transmembrane and cytoplasmic domain of the MHC molecule comprises the amino acid sequence IVGIVAGLAVLAVVVIGAVVATVMCRRKSSGGKGGSYSQAASSDSAQGSDVSLTA (SEQ ID NO: 30). In some embodiments, the polynucleotide sequence encoding the at least portion of the transmembrane and cytoplasmic domain of the MHC molecule comprises the sequence

(SEQ ID NO: 28)
AUCGUGGGAAUUGUGGCAGGACUGGCAGUGCUGGCCGUGGUGGUG
AUCGGAGCCGUGGUGGCUACCGUGAUGUGCAGACGGAAGUCCAGC
GGAGGCAAGGGCGGCAGCUACAGCCAGGCCGCCAGCUCUGAUAGC
GCCCAGGGCAGCGACGUGUCACUGACAGCC.

In some embodiments, the 3′ untranslated region of the AES mRNA comprises the sequence

(SEQ ID NO: 33)
CUGGUACUGCAUGCACGCAAUGCUAGCUGCCCCUUUCCCGUCCUG
GGUACCCCGAGUCUCCCCCGACCUCGGGUCCCAGGUAUGCUCCCA
CCUCCACCUGCCCCACUCACCACCUCUGCUAGUUCCAGACACCUC
C.

In some embodiments, the non-coding RNA of the mitochondrially encoded 12S RNA comprises the sequence

(SEQ ID NO: 35)
CAAGCACGCAGCAAUGCAGCUCAAAACGCUUAGCCUAGCCACACC
CCCACGGGAAACAGCAGUGAUUAACCUUUAGCAAUAAACGAAAGU
UUAACUAAGCUAUACUAACCCCAGGGUUGGUCAAUUUCGUGCCAG
CCACACCG.

In some embodiments, the 3′ UTR comprises the sequence

(SEQ ID NO: 31)
CUCGAGCUGGUACUGCAUGCACGCAAUGCUAGCUGCCCCUUUCCC
GUCCUGGGUACCCCGAGUCUCCCCCGACCUCGGGUCCCAGGUAUG
CUCCCACCUCCACCUGCCCCACUCACCACCUCUGCUAGUUCCAGA
CACCUCCCAAGCACGCAGCAAUGCAGCUCAAAACGCUUAGCCUAG
CCACACCCCCACGGGAAACAGCAGUGAUUAACCUUUAGCAAUAAA
CGAAAGUUUAACUAAGCUAUACUAACCCCAGGGUUGGUCAAUUUC
GUGCCAGCCACACCGAGACCUGGUCCAGAGUCGCUAGCCGCGUCG
CU.

In some embodiments, the poly(A) sequence comprises 120 adenine nucleotides.

In some embodiments, the RNA vaccine comprises an RNA molecule comprising, in the 5′→3′ direction: the polynucleotide sequence GGCGAACUAGUAUUCUUCUGGUCCCCACAGACUCAGAGAGAACCCGCCACCAUGAGAG UGAUGGCCCCCAGAACCCUGAUCCUGCUGCUGUCUGGCGCCCUGGCCCUGACAGAGAC AUGGGCCGGAAGC (SEQ ID NO: 19); a polynucleotide sequence encoding the one or more neoepitopes resulting from cancer-specific somatic mutations present in the tumor specimen; and the polynucleotide sequence

(SEQ ID NO: 20)
AUCGUGGGAAUUGUGGCAGGACUGGCAGUGCUGGCCGUGGUGGUGAUCGG
AGCCGUGGUGGCUACCGUGAUGUGCAGACGGAAGUCCAGCGGAGGCAAGG
GCGGCAGCUACAGCCAGGCCGCCAGCUCUGAUAGCGCCCAGGGCAGCGAC
GUGUCACUGACAGCCUAGUAACUCGAGCUGGUACUGCAUGCACGCAAUGC
UAGCUGCCCCUUUCCCGUCCUGGGUACCCCGAGUCUCCCCCGACCUCGGG
UCCCAGGUAUGCUCCCACCUCCACCUGCCCCACUCACCACCUCUGCUAGU
UCCAGACACCUCCCAAGCACGCAGCAAUGCAGCUCAAAACGCUUAGCCUA
GCCACACCCCCACGGGAAACAGCAGUGAUUAACCUUUAGCAAUAAACGAA
AGUUUAACUAAGCUAUACUAACCCCAGGGUUGGUCAAUUUCGUGCCAGCC
ACACCGAGACCUGGUCCAGAGUCGCUAGCCGCGUCGCU.

In some embodiments, the pancreatic cancer tumor is a resectable PDAC tumor, assessed by preoperative imaging in the patient with computed tomography (CT) scan with contrast or magnetic resonance imaging (MRI) prior to administration of the RNA vaccine, the PD-1 axis binding antagonist, and the chemotherapeutic treatment. In some embodiments, the pancreatic cancer tumor is a resectable PDAC tumor comprising one or more characteristics selected from the group consisting of: clear fat plane around the celiac and superior mesenteric arteries; patent superior mesenteric and portal veins; no encasement of the superior mesenteric vein or portal veins; no encasement of the superior mesenteric or hepatic arteries; absence of metastatic disease; and absence of extra-regional nodal disease. In some embodiments, the patient has a histologically confirmed diagnosis of PDAC prior to administration of the RNA vaccine, the PD-1 axis binding antagonist, and the chemotherapeutic treatment.

In some embodiments, the patient has adenosquamous carcinoma of the pancreas prior to administration of the RNA vaccine, the PD-1 axis binding antagonist, and the chemotherapeutic treatment.

In some embodiments, the pancreatic cancer tumor has tumor, lymph node, metastasis (TNM) pathological staging values of T1-T3, NO-N2, or MO prior to administration of the RNA vaccine, the PD-1 axis binding antagonist, and the chemotherapeutic treatment.

In some embodiments, the pancreatic cancer tumor is a resectable PDAC tumor, and wherein: the patient had no evidence of PDAC disease after resection of the PDAC tumor, and/or the patient had a macroscopically complete resection of the PDAC tumor, prior to administration of the RNA vaccine, the PD-1 axis binding antagonist, and the chemotherapeutic treatment, optionally wherein the patient had a RO or RI resection of the PDAC tumor. In some embodiments, the patient had unequivocal absence of PDAC after resection of the PDAC tumor, optionally wherein the absence of PDAC is assessed by CT or MRI scans, one or more biochemical assays and/or clinical findings. In some embodiments, the pancreatic cancer tumor is a resectable PDAC tumor, and wherein following resection of the tumor, the patient did not have unresolved >Grade 3 postoperative complications prior to administration of the RNA vaccine, the PD-1 axis binding antagonist, and the chemotherapeutic treatment, optionally wherein the complications are assessed according to the Clavien Dindo Classification of Surgical Complications.

In some embodiments, the patient has a CA19-9 level of 180 U/mL or greater prior to administration of the RNA vaccine, the PD-1 axis binding antagonist, and the chemotherapeutic treatment. In some embodiments, the patient has a CA19-9 level of less than 180 U/mL prior to administration of the RNA vaccine, the PD-1 axis binding antagonist, and the chemotherapeutic treatment.

In some embodiments, at least five neoepitopes resulting from cancer-specific somatic mutations are present in the tumor specimen obtained from the patient prior to administration of the RNA vaccine, the PD-1 axis binding antagonist, and the chemotherapeutic treatment.

In some embodiments, the patient has an Eastern Cooperative Oncology Group (ECOG) Performance Status of 0 or 1 prior to administration of the RNA vaccine, the PD-1 axis binding antagonist, and the chemotherapeutic treatment.

In some embodiments, the patient does not have intraductal papillary mucinous neoplasm-associated PDAC prior to administration of the RNA vaccine, the PD-1 axis binding antagonist, and the chemotherapeutic treatment.

In some embodiments, the patient does not have a pancreatic endocrine tumor or acinar cell adenocarcinoma, pancreatic cystadenocarcinoma, or pancreatic malignant ampulloma prior to administration of the RNA vaccine, the PD-1 axis binding antagonist, and the chemotherapeutic treatment.

In some embodiments, the patient has not received an adjuvant, neoadjuvant, or induction treatment for pancreatic cancer, or a systemic anti-cancer treatment for pancreatic cancer, prior to administration of the RNA vaccine, the PD-1 axis binding antagonist, and the chemotherapeutic treatment; optionally wherein the pancreatic cancer is PDAC. In some embodiments, the patient has not had a cytotoxic chemotherapy, immunotherapy, investigational therapy, or radiation therapy prior to administration of the RNA vaccine, the PD-1 axis binding antagonist, and the chemotherapeutic treatment.

In some embodiments, the patient has a spleen prior to administration of the RNA vaccine, the PD-1 axis binding antagonist, and the chemotherapeutic treatment. In some embodiments, the patient has not had loss of spleen due to splenectomy, splenic injury/infarction, or functional asplenia prior to administration of the RNA vaccine, the PD-1 axis binding antagonist, and the chemotherapeutic treatment. In some embodiments, the patient has not had a distal pancreatectomy with splenectomy prior to administration of the RNA vaccine, the PD-1 axis binding antagonist, and the chemotherapeutic treatment.

In some embodiments, the patient does not have preexisting neuropathy prior to administration of the RNA vaccine, the PD-1 axis binding antagonist, and the chemotherapeutic treatment.

In some embodiments, the patient does not have an aUGTIAl genotype associated with poor metabolizer phenotype prior to administration of the RNA vaccine, the PD-1 axis binding antagonist, and the chemotherapeutic treatment.

In some embodiments, the patient does not have an autoimmune disease, immune deficiency, or primary immunodeficiency prior to administration of the RNA vaccine, the PD-1 axis binding antagonist, and the chemotherapeutic treatment. In some embodiments, the patient has not been treated with: monoamine oxidase inhibitors (MAOIs) within 3 weeks, a systemic immunostimulatory agent within 4 weeks or 5 drug-elimination half-lives, whichever is longer, or a systemic immunosuppressive medication within 2 weeks, prior to administration of the RNA vaccine, the PD-1 axis binding antagonist, and the chemotherapeutic treatment.

In some embodiments, the patient has not had an allogeneic stem cell or solid organ transplantation prior to administration of the RNA vaccine, the PD-1 axis binding antagonist, and the chemotherapeutic treatment.

In some embodiments, the method further comprises assessing disease-free survival (DFS) of the patient after treatment with the RNA vaccine, the PD-1 axis binding antagonist, and the chemotherapeutic treatment. In some embodiments, administration of the RNA vaccine, the PD-1 axis binding antagonist, and the chemotherapeutic treatment results in an improvement in DFS of the patient as compared to DFS of a corresponding patient not administered the RNA vaccine, the PD-1 axis binding antagonist, and the chemotherapeutic treatment.

In some embodiments, the method further comprises assessing overall survival (OS) of the patient after treatment with the RNA vaccine, the PD-1 axis binding antagonist, and the chemotherapeutic treatment. In some embodiments, administration of the RNA vaccine, the PD-1 axis binding antagonist, and the chemotherapeutic treatment results in an improvement in OS of the patient as compared to OS of a corresponding patient not administered the RNA vaccine, the PD-1 axis binding antagonist, and the chemotherapeutic treatment.

In some embodiments, the method further comprises performing one or more clinical assessments of the patient before, during and/or after treatment with the RNA vaccine, the PD-1 axis binding antagonist, and the chemotherapeutic treatment, wherein the one or more clinical assessments are selected from the group consisting of European Organisation for Research and Treatment of Cancer QLQ-C30 Questionnaire (EORTC QLQ C30), European Organisation for Research and Treatment of Cancer QLQ-PAN26 Questionnaire (EORTC QLQ PAN26), National Cancer Institute's Patient-Reported Outcomes Common Terminology Criteria for Adverse Events (PRO CTCAE), and European Organisation for Research and Treatment of Cancer Item Library 46 Questionnaire (EORTC IL46). In some embodiments, administration of the RNA vaccine, the PD-1 axis binding antagonist, and the chemotherapeutic treatment results in an improvement in the one or more clinical assessments as compared to the one or more clinical assessments in the patient prior to administration of the RNA vaccine, the PD-1 axis binding antagonist, and the chemotherapeutic treatment, and/or as compared to the one or more clinical assessments in a corresponding patient not administered the RNA vaccine, the PD-1 axis binding antagonist, and the chemotherapeutic treatment.

In some embodiments, the method further comprises assessing antigen- and/or tumor-specific T-cell responses in the patient before, during and/or after treatment with the RNA vaccine, the PD-1 axis binding antagonist, and the chemotherapeutic treatment. In some embodiments, administration of the RNA vaccine, the PD-1 axis binding antagonist, and the chemotherapeutic treatment results in an improvement in antigen- and/or tumor-specific T-cell responses in the patient as compared to prior to administration of the RNA vaccine, the PD-1 axis binding antagonist, and the chemotherapeutic treatment, and/or as compared to a corresponding patient not administered the RNA vaccine, the PD-1 axis binding antagonist, and the chemotherapeutic treatment.

In some embodiments, the corresponding patient is a patient with a corresponding pancreatic cancer tumor, optionally wherein the pancreatic cancer tumor is a PDAC tumor, and the corresponding patient has a PDAC tumor. In some embodiments, the corresponding patient was treated with a standard of care treatment for pancreatic cancer, PDAC, or resectable or resected PDAC. In some embodiments, the standard of care treatment comprises a gemcitabine combination therapy or an mFOLFIRINOX chemotherapy.

In some embodiments, the corresponding patient was treated with a control treatment comprising an mFOLFIRINOX chemotherapy. In some embodiments, the mFOLFIRINOX chemotherapy comprises oxaliplatin at dose of about 85 mg/m², leucovorin at dose of about 400 mg/m², irinotecan at dose of about 150 mg/m², and 5-fluorouracil at dose of about 2400 mg/m², administered intravenously in 14-day cycles, on day 1 of each cycle for a total of up to 12 cycles.

In one aspect is provided an individualized RNA vaccine for use in a method for treating a pancreatic cancer tumor in a human patient in need thereof, wherein the RNA vaccine is to be administered in combination with a PD-1 axis binding antagonist and a chemotherapeutic treatment according to the method described herein, wherein the RNA vaccine comprises one or more polynucleotides encoding one or more neoepitopes resulting from cancer-specific somatic mutations present in a pancreatic cancer tumor specimen obtained from the patient. In another aspect is provided a PD-1 axis binding antagonist for use in a method for treating a pancreatic cancer tumor in a human patient in need thereof, wherein the PD-1 axis binding antagonist is to be administered in combination with an individualized RNA vaccine and a chemotherapeutic treatment according to the method described herein, wherein the RNA vaccine comprises one or more polynucleotides encoding one or more neoepitopes resulting from cancer-specific somatic mutations present in a pancreatic cancer tumor specimen obtained from the patient.

In one aspect is provided a use of an individualized RNA vaccine in the manufacture of a medicament for treating a pancreatic cancer tumor in a human patient in need thereof, wherein the RNA vaccine is to be administered in combination with a PD-1 axis binding antagonist and a chemotherapeutic treatment according to the method described herein, and wherein the RNA vaccine comprises one or more polynucleotides encoding one or more neoepitopes resulting from cancer-specific somatic mutations present in a pancreatic cancer tumor specimen obtained from the patient. In another aspect is provided a use of a PD-1 axis binding antagonist in the manufacture of a medicament for treating a pancreatic cancer tumor in a human patient in need thereof, wherein the PD-1 axis binding antagonist is to be administered in combination with an individualized RNA vaccine and a chemotherapeutic treatment according to the method described herein, and wherein the RNA vaccine comprises one or more polynucleotides encoding one or more neoepitopes resulting from cancer-specific somatic mutations present in a pancreatic cancer tumor specimen obtained from the patient.

In one aspect is provided a kit comprising an individualized RNA vaccine, for use in a method for treating a pancreatic cancer tumor in a human patient in need thereof, wherein the RNA vaccine is to be administered in combination with a PD-1 axis binding antagonist and a chemotherapeutic treatment according to the method described herein, wherein the RNA vaccine comprises one or more polynucleotides encoding one or more neoepitopes resulting from cancer-specific somatic mutations present in a pancreatic cancer tumor specimen obtained from the patient. In another aspect is provided a kit comprising a PD-1 axis binding antagonist for use in a method for treating a pancreatic cancer tumor in a human patient in need thereof, wherein the PD-1 axis binding antagonist is to be administered in combination with an individualized RNA vaccine and a chemotherapeutic treatment according to the method described herein, wherein the RNA vaccine comprises one or more polynucleotides encoding one or more neoepitopes resulting from cancer-specific somatic mutations present in a pancreatic cancer tumor specimen obtained from the patient.

In one aspect is provided a method of selecting a human patient having a cancer tumor as likely to respond to a therapy comprising an individualized RNA vaccine, the method comprising: a) measuring the number and/or frequency of de novo significantly expanded (SE) TCR clones in a sample from the patient by T cell receptor sequencing; b) comparing the number and/or frequency of de novo SE TCR clones measured in a) to a reference number and/or frequency; and c) selecting the patient as more likely to respond to the therapy comprising the individualized RNA vaccine when the number and/or frequency of de novo SE TCR clones in the sample from the patient is above the reference number and/or frequency; wherein the individualized RNA vaccine comprises one or more polynucleotides encoding one or more neoepitopes resulting from cancer-specific somatic mutations present in a cancer tumor specimen obtained from the patient, and wherein a number and/or frequency of de novo SE TCR clones above the reference number and/or frequency indicates that the patient is more likely to respond to the therapy comprising the individualized RNA vaccine. In some embodiments, the method further comprises selecting the therapy comprising the individualized RNA vaccine or recommending a therapy comprising the individualized RNA vaccine. In another aspect is provided a method of selecting a human patient having a cancer tumor as likely to respond to a therapy comprising an individualized RNA vaccine, the method comprising: a) comparing the number and/or frequency of de novo significantly expanded (SE) TCR clones in a sample from the patient to a reference number and/or frequency; and b) selecting the patient as more likely to respond to the therapy comprising the individualized RNA vaccine when the number and/or frequency of de novo SE TCR clones in the sample from the patient is above the reference number and/or frequency; wherein the individualized RNA vaccine comprises one or more polynucleotides encoding one or more neoepitopes resulting from cancer-specific somatic mutations present in a cancer tumor specimen obtained from the patient, wherein the number and/or frequency of de novo SE TCR clones is measured by T cell receptor sequencing, and wherein a number and/or frequency of de novo SE TCR clones above the reference number and/or frequency indicates that the patient is more likely to respond to the therapy comprising the individualized RNA vaccine.

In one aspect is provided a method of treating a human patient having a cancer tumor, the method comprising: a) measuring the number and/or frequency of de novo significantly expanded (SE) TCR clones in a sample from the patient by T cell receptor sequencing; b) comparing the number and/or frequency of de novo SE TCR clones measured in a) to a reference number and/or frequency; and c) selecting the patient as more likely to respond to the therapy comprising the individualized RNA vaccine when the number and/or frequency of de novo SE TCR clones in the sample from the patient is above the reference number and/or frequency; wherein the individualized RNA vaccine comprises one or more polynucleotides encoding one or more neoepitopes resulting from cancer-specific somatic mutations present in a cancer tumor specimen obtained from the patient, and wherein a number and/or frequency of de novo SE TCR clones above the reference number and/or frequency indicates that the patient is more likely to respond to the therapy comprising the individualized RNA vaccine. In another aspect is provided a method of treating a human patient having a cancer tumor, the method comprising: a) comparing the number and/or frequency of de novo significantly expanded (SE) TCR clones in a sample from the patient to a reference number and/or frequency; and b) selecting the patient as more likely to respond to the therapy comprising the individualized RNA vaccine when the number and/or frequency of de novo SE TCR clones in the sample from the patient is above the reference number and/or frequency, thereby treating the cancer tumor; wherein the individualized RNA vaccine comprises one or more polynucleotides encoding one or more neoepitopes resulting from cancer-specific somatic mutations present in a cancer tumor specimen obtained from the patient, wherein the number and/or frequency of de novo SE TCR clones is measured by T cell receptor sequencing, and wherein a number and/or frequency of de novo SE TCR clones above the reference number and/or frequency indicates that the patient is more likely to respond to the therapy comprising the individualized RNA vaccine.

In some embodiments, the method further comprises administering the therapy comprising the individualized RNA vaccine to the patient when the number and/or frequency of de novo SE TCR clones in the sample from the patient is above the reference number and/or frequency, thereby treating the cancer tumor. In some embodiments, the method further comprises selecting the therapy comprising the individualized RNA vaccine when the number and/or frequency of de novo SE TCR clones in the sample from the patient is above the reference number and/or frequency, thereby treating the cancer tumor. In some embodiments, the number and/or frequency is measured after six doses of the individualized cancer vaccine. In some embodiments, the reference number is six de novo SE TCR clones. In some embodiments, the reference frequency is 10⁻⁴ de novo SE TCR clones.

In some embodiments, the cancer tumor is a pancreatic cancer tumor. In some embodiments, the cancer tumor is a pancreatic ductal adenocarcinoma (PDAC) tumor.

In some embodiments, the therapy comprising the individualized RNA vaccine further comprises a PD-1 axis binding antagonist. In some embodiments, the therapy further comprises a chemotherapeutic treatment, wherein the RNA vaccine, the PD-1 axis binding antagonist, and the chemotherapeutic treatment are administered to the patient during a priming phase, a chemotherapy phase after the priming phase, and a boost phase after the chemotherapy phase, wherein: (i) the priming phase comprises administering to the patient at least one dose of the RNA vaccine and at least one dose of the PD-1 axis binding antagonist, (ii) the chemotherapy phase comprises administering to the patient the chemotherapeutic treatment, and (iii) the boost phase comprises administering to the patient at least one dose of the RNA vaccine and at least one dose of the PD-1 axis binding antagonist. In some embodiments, the PD-1 axis binding antagonist is atezolizumab. In some embodiments, the chemotherapeutic treatment is a FOLFIRINOX treatment or an mFOLFIRINOX treatment.

In some embodiments, prior to the administering step, the patient is selected by a method comprising: a) measuring the number and/or frequency of de novo significantly expanded (SE) TCR clones in a sample from the patient by T cell receptor sequencing; b) comparing the number and/or frequency of de novo SE TCR clones measured in a) to a reference number and/or frequency; and c) selecting the patient as more likely to respond to the therapy comprising the individualized RNA vaccine when the number and/or frequency of de novo SE TCR clones in the sample from the patient is above the reference number and/or frequency; wherein the individualized RNA vaccine comprises one or more polynucleotides encoding one or more neoepitopes resulting from cancer-specific somatic mutations present in a cancer tumor specimen obtained from the patient, and wherein a number and/or frequency of de novo SE TCR clones above the reference number and/or frequency indicates that the patient is more likely to respond to the therapy comprising the individualized RNA vaccine.

In one aspect is provided an in vitro use of number and/or frequency of de novo significantly expanded (SE) TCR clones for selecting a patient having a cancer tumor more likely to respond to a therapy comprising an individualized RNA vaccine, wherein a number and/or frequency of de novo SE TCR clones in the sample from the patient above a reference number and/or frequency selects that the patient is more likely to respond to the therapy comprising the individualized RNA vaccine.

In one aspect is provided a use of a number and/or frequency of de novo significantly expanded (SE) TCR clones for the manufacture of a diagnostic for assessing the likelihood of a response of a patient having a cancer tumor to a therapy comprising an individualized RNA vaccine.

### BRIEF DESCRIPTION OF THE DRAWINGS

FIG. 1 provides a schematic of the design of the Phase II study described in Example 1. Patients with resectable PDAC undergo screening parts A and B, then undergo randomization into one of two arms: arm 1 wherein patients are administered the individualized RNA vaccine in combination with atezolizumab and mFOLFIRINOX, and arm 2 wherein patients are administered mFOLFIRINOX alone. mFOLFIRINOX: modified leucovorin, 5 fluorouracil (5-FU), irinotecan, oxaliplatin; PDAC: pancreatic ductal adenocarcinoma; Q4W: every 4 weeks.

FIGS. 2A-2B provide diagrams of the design of the study treatment phases and dosing schedules for arms 1 and 2 of the study described in Example 1. FIG. 2A provides a diagram of the design of the study treatment phases and dosing schedules of dosing regimen A for arms 1 and 2. Subjects in arm 1, dosing regimen A, of the Phase II study are administered the individualized RNA vaccine (at a dose of 25 μg once a week for six weeks by IV infusion) in combination with atezolizumab (administration on Day 1 of Weeks 1 and 5 at a dose of 1680 mg by IV infusion) in the Priming Phase; mFOLFIRINOX (administration in 14-day cycles starting Week 7 for 12 rounds) in the Chemotherapy Phase; and RNA vaccine (administration in 28-day cycles starting Week 33 for six rounds at a dose of 25 μg by IV infusion) in combination with atezolizumab (administration in 28-day cycles starting Week 33 for six rounds at a dose of 1680 mg by IV infusion) in the Boost Phase. Subjects in arm 2 of the Phase II study are administered mFOLFIRINOX alone in 14-day cycles starting Week 1 for a total of up to 12 rounds. FIG. 2B provides a diagram of the design of the study treatment phases and dosing schedules of dosing regimen B for arms 1 and 2. Subjects in arm 1, dosing regimen B, of the Phase II study are administered the individualized RNA vaccine (at a dose of 25 μg once a week for six weeks by IV infusion) in combination with atezolizumab (administration on Day 1 of Week 3 at a dose of 1680 mg by IV infusion) in the Priming Phase; mFOLFIRINOX (administration in 14-day cycles starting Week 7 for 12 rounds) in the Chemotherapy Phase; and RNA vaccine (administration in 28-day cycles starting Week 33 for six rounds at a dose of 25 μg by IV infusion) in combination with atezolizumab (administration in 28-day cycles starting Week 33 for six rounds at a dose of 1680 mg by IV infusion) in the Boost Phase. Subjects in arm 2 of the Phase II study are administered mFOLFIRINOX alone in 14-day cycles starting Week 1 for a total of up to 12 rounds. In FIGS. 2A-2B, B: boost; C: chemotherapy; mFOLFIRINOX: modified leucovorin, 5 fluorouracil (5-FU), irinotecan, oxaliplatin; P: priming.

FIG. 3 provides a relative schedule of RNA vaccine and chemotherapy combination therapy in a murine syngeneic MC38 tumor model. The chemotherapy regimen involved intraperitoneal injection (i.p.) of modified mouse FOLFIRINOX. The RNA vaccine (referred to as RNA-LPX) involved three doses administered by intravenous injection (i.v.) on days 0, 7, and 14 (gray arrows). The chemotherapy regimen was started at various times (black arrows) relative to the RNA vaccine as described in Example 2. Blood was collected from n=5 mice per group each week to analyze T cell responses. Deca: “Decatope”; LPX: lipoplex; FOLFIRINOX: leucovorin, 5 fluorouracil (5-FU), irinotecan, oxaliplatin. n=10 mice per group.

FIG. 4 provides a line graph showing the tumor growth curve for each of 8 treatment groups. LPX: lipoplex; IV: intravenous injection; Q14Dx3: once every 14 days for three rounds; QWx3: once every week for three rounds; FOLFIRINOX: leucovorin, 5 fluorouracil (5-FU), irinotecan, oxaliplatin. n=10 mice per group.

FIG. 5 provides line graphs that show the tumor growth curve for each individual mouse in a treatment group with the group best-fit curve and the reference fit overlaid on the graphs. Each graph represents the results from one of the 8 treatment groups. Black arrows indicate the time points when mice received chemotherapy treatment. Gray arrows represent the time point when mice received Decatope 1+2 RNA-LPX vaccination. Reference fit and group fit curves are indicated with labeled arrows. LPX: lipoplex; IV: intravenous injection; Q14Dx3: once every 14 days for three rounds; QWx3: once every week for three rounds; FOLFIRINOX: leucovorin, 5 fluorouracil (5-FU), irinotecan, oxaliplatin. n=10 mice per group.

FIG. 6 is a diagram of the design of the Phase Ia/Ib study described in Example 3. Subjects in the Phase Ia dose escalation study were administered the RNA vaccine as a monotherapy at doses of 25 μg, 38 μg, 50 μg, 75 μg, or 100 μg. During initial treatment (induction stage), the RNA vaccine was administered on Days 1, 8, and 15 of Cycle 1, on Days 1, 8 and 15 of Cycle 2, on Days 1 and 15 of Cycle 3, and on Day 1 of Cycle 7 (each cycle was 21 days). During the maintenance stage after initial treatment, the RNA vaccine was administered on Day 1 of Cycle 13, and every 8 cycles thereafter (i.e., every 24 weeks thereafter, or every 168 days thereafter) until disease progression (PD) (each cycle was 21 days). Subjects in the Phase Ib study were administered the RNA vaccine in doses of 15 μg (not shown), 25 μg, 38 μg, or 50 μg in combination with 1200 mg atezolizumab. The Phase Ib study included a dose escalation phase for the RNA vaccine and an expansion phase in which patients with the indicated checkpoint inhibitor naïve or checkpoint inhibitor experienced tumor types were administered the RNA vaccine at a dose of 15 μg or 25 μg in combination with atezolizumab. During initial treatment (induction stage), atezolizumab was administered on Day 1 of each of Cycles 1-12; and the RNA vaccine was administered on Days 1, 8, and 15 of Cycle 1, on Days 1, 8 and 15 of Cycle 2, on Days 1 and 15 of Cycle 3, and on Day 1 of Cycle 7 (each cycle was 21 days). During the maintenance stage after initial treatment, atezolizumab was administered every 3 weeks until disease progression (PD), starting on Day 1 of Cycle 13; and the RNA vaccine was administered on Day 1 of Cycle 13, and every 8 cycles thereafter (i.e., every 24 weeks thereafter, or every 168 days thereafter) until disease progression (PD) (each cycle was 21 days).

FIG. 7 provides a diagram of the MHC multimer staining assays used to evaluate neoantigen-specific CD8+ T cell immune responses following administration of the RNA vaccine as a monotherapy (Phase Ia) or in combination with atezolizumab (Phase Ib), as described in Example 3 herein.

FIGS. 8A-8B show the results of MHC multimer staining assays that evaluated neoantigen-specific CD8+ T cell immune responses following treatment with an individualized RNA vaccine alone or combined with atezolizumab according to the Phase Ia/Ib study described in Example 3 herein. FIG. 8A shows the frequency of antigen-specific CD8+ T cells in patients in the Phase Ia study administered the individualized RNA vaccine as a monotherapy. The individualized RNA vaccine dosing regimen is shown below the plot. FIG. 8B shows the frequency of antigen-specific CD8+ T cells in patients in the Phase Ib study administered the individualized RNA vaccine combined with atezolizumab. The individualized RNA vaccine and atezolizumab (“Atezo”) dosing regimen is shown below the plot. In FIGS. 8A-8B, each line represents a unique antigen specific CD8+ T cell response measured longitudinally. C, cycle.

FIG. 9 shows the results of MHC multimer staining assays that evaluated neoantigen-specific CD8+ T cell immune responses following a boost dose of individualized RNA vaccine according to the Phase Ia/Ib study described in Example 3 herein. The frequency of antigen-specific CD8+ T cells pre-boost at Cycle 6, Day 1 (C6D1) or Cycle 12, Day 1 (C12D1), and post-boost at Cycle 8, Day 1 (C8D1) or Cycle 14, Day 1 (C14D1) is shown. Timing of boost dose for the individualized RNA vaccine is shown by the arrows and text below the plot (Cycle 7, Day 1 [C7D1], and Cycle 13, Day 1 [C13D1]). Each line represents a unique antigen specific CD8+ T cell response measured longitudinally.

FIG. 10 shows the schematic of significantly expanded T cell receptor (TCR) clones in one sample to identify expanded clones upon vaccination. Each dot represents a unique TCR clone, identified based on the nucleotide sequence, and the frequency of that clone in the pre-treatment (i.e., baseline) sample and in the on-treatment sample after administration of 6 doses of vaccine is plotted in Log10 scale on the x and y axes, respectively. The points are colored based on the significance status, as the frequency of two time points were compared in a statistical beta-binomial model based on an adjusted p-value of 0.01. The significantly expanded (SE) clones are colored in black and labeled as de novo, if the clone was not detected in the baseline sample, or as pre-existing if the clone was detected in the baseline but the frequency was expanded post vaccination. The treatment schedule of vaccine and Atezo is also plotted for this patient.

FIG. 11 shows the higher number of de novo significantly expanded (SE) clones in the immune responders to the vaccine (i.e., ELISpot positive). The number of expanded TCR clones at different stages of treatment: 3-Vax (after about 3 doses of vaccine), 6-Vax (after about 6 doses of vaccine), 8-Vax (after about 8 doses of vaccine), and 9/10-Vax (after about 9 or 10 doses of vaccine) are plotted for each patient. Each dot represents data from a single patient at the indicated stage of vaccination. The immunogenicity of patients was assessed by the ELISpot assay. The p-values are based on Wilcoxon's Mann-Whitney test.

### DETAILED DESCRIPTION

### I. Definitions

Before describing the invention in detail, it is to be understood that this invention is not limited to particular compositions or biological systems, which can, of course, vary. It is also to be understood that the terminology used herein is for the purpose of describing particular embodiments only, and is not intended to be limiting.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

The term “PD-1 axis binding antagonist” refers to a molecule that inhibits the interaction of a PD-1 axis binding partner with either one or more of its binding partner, so as to remove T-cell dysfunction resulting from signaling on the PD-1 signaling axis—with a result being to restore or enhance T-cell function (e.g., proliferation, cytokine production, target cell killing). As used herein, a PD-1 axis binding antagonist includes a PD-1 binding antagonist, a PD-L1 binding antagonist and a PD-L2 binding antagonist.

The term “PD-1 binding antagonist” refers to a molecule that decreases, blocks, inhibits, abrogates or interferes with signal transduction resulting from the interaction of PD-1 with one or more of its binding partners, such as PD-L1, PD-L2. In some embodiments, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In one embodiment, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some embodiments, the PD-1 binding antagonist is an anti-PD-1 antibody. Specific examples of PD-1 binding antagonists are provided infra.

The term “PD-L1 binding antagonist” refers to a molecule that decreases, blocks, inhibits, abrogates or interferes with signal transduction resulting from the interaction of PD-L1 with either one or more of its binding partners, such as PD-1, B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, the PD-L1 binding antagonists include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1, B7-1. In one embodiment, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some embodiments, a PD-L1 binding antagonist is an anti-PD-L1 antibody. Specific examples of PD-L1 binding antagonists are provided infra.

The term “PD-L2 binding antagonist” refers to a molecule that decreases, blocks, inhibits, abrogates or interferes with signal transduction resulting from the interaction of PD-L2 with either one or more of its binding partners, such as PD-1. In some embodiments, a PD-L2 binding antagonist is a molecule that inhibits the binding of PD-L2 to one or more of its binding partners. In a specific aspect, the PD-L2 binding antagonist inhibits binding of PD-L2 to PD-1. In some embodiments, the PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one embodiment, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some embodiments, a PD-L2 binding antagonist is an immunoadhesin.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration at least the same as the treatment duration, at least 1.5×, 2.0×, 2.5×, or 3.0× length of the treatment duration.

The term “pharmaceutical formulation” refers to a preparation which is in such form as to permit the biological activity of the active ingredient to be effective, and which contains no additional components which are unacceptably toxic to a subject to which the formulation would be administered. Such formulations are sterile. “Pharmaceutically acceptable” excipients (vehicles, additives) are those which can reasonably be administered to a subject mammal to provide an effective dose of the active ingredient employed.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of individuals.

As used herein, “delaying progression of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late-stage cancer, such as development of metastasis, may be delayed.

An “effective amount” is at least the minimum amount required to effect a measurable improvement or prevention of a particular disorder. An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, “in conjunction with” or “in combination with” refers to administration of one treatment modality in addition to another treatment modality. As such, “in conjunction with” or “in combination with” refers to administration of one treatment modality before, during, or after administration of the other treatment modality to the individual.

A “disorder” is any condition that would benefit from treatment including, but not limited to, chronic and acute disorders or diseases including those pathological conditions which predispose the mammal to the disorder in question.

The terms “cell proliferative disorder” and “proliferative disorder” refer to disorders that are associated with some degree of abnormal cell proliferation. In one embodiment, the cell proliferative disorder is cancer. In one embodiment, the cell proliferative disorder is a tumor.

“Tumor,” as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder” and “tumor” are not mutually exclusive as referred to herein.

A “subject”, “patient” or an “individual” for purposes of treatment refers to any animal classified as a mammal, including humans, domestic and farm animals, and zoo, sports, or pet animals, such as dogs, horses, cats, cows, etc. Preferably, the mammal is human.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

An “isolated” antibody is one which has been identified and separated and/or recovered from a component of its natural environment. Contaminant components of its natural environment are materials which would interfere with research, diagnostic or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. Isolated antibody includes the antibody in situ within recombinant cells since at least one component of the antibody's natural environment will not be present. Ordinarily, however, isolated antibody will be prepared by at least one purification step.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The term “constant domain” refers to the portion of an immunoglobulin molecule having a more conserved amino acid sequence relative to the other portion of the immunoglobulin, the variable domain, which contains the antigen binding site. The constant domain contains the CH1, CH2 and CH3 domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The “light chains” of antibodies (immunoglobulins) from any mammalian species can be assigned to one of two clearly distinct types, called kappa (“K”) and lambda (“2”), based on the amino acid sequences of their constant domains.

The term IgG “isotype” or “subclass” as used herein is meant any of the subclasses of immunoglobulins defined by the chemical and antigenic characteristics of their constant regions.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The terms “full length antibody,” “intact antibody” and “whole antibody” are used herein interchangeably to refer to an antibody in its substantially intact form, not antibody fragments as defined below. The terms particularly refer to an antibody with heavy chains that contain an Fc region.

A “naked antibody” for the purposes herein is an antibody that is not conjugated to a cytotoxic moiety or radiolabel.

“Antibody fragments” comprise a portion of an intact antibody, preferably comprising the antigen binding region thereof. In some embodiments, the antibody fragment described herein is an antigen-binding fragment. Examples of antibody fragments include Fab, Fab′, F(ab′) 2, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′) 2 fragment that has two antigen-combining sites and is still capable of cross-linking antigen.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CHI domain including one or more cysteines from the antibody hinge region. Fab′-SH is the designation herein for Fab′ in which the cysteine residue(s) of the constant domains bear a free thiol group. F(ab′) 2 antibody fragments originally were produced as pairs of Fab′ fragments which have hinge cysteines between them. Other chemical couplings of antibody fragments are also known.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv, see, e.g., Pluckthün, in The Pharmacology of Monoclonal Antibodies, vol. 113, Rosenburg and Moore eds., (Springer-Verlag, New York, 1994), pp. 269-315.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al., Proc. Natl. Acad. Sci. USA 90:6444-6448 (1993). Triabodies and tetrabodies are also described in Hudson et al., Nat. Med. 9:129-134 (2003).

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352:624-628 (1991); Marks et al., J. Mol. Biol. 222:581-597 (1992); Sidhu et al., J. Mol. Biol. 338 (2): 299-310 (2004); Lee et al., J. Mol. Biol. 340 (5): 1073-1093 (2004); Fellouse, Proc. Natl. Acad. Sci. USA 101 (34): 12467-12472 (2004); and Lee et al., J. Immunol. Methods 284 (1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90:2551 (1993); Jakobovits et al., Nature 362:255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10:779-783 (1992); Lonberg et al., Nature 368:856-859 (1994); Morrison, Nature 368:812-813 (1994); Fishwild et al., Nature Biotechnol. 14:845-851 (1996); Neuberger, Nature Biotechnol. 14:826 (1996); and Lonberg and Huszar, Intern. Rev. Immunol. 13:65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATTZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR of the recipient are replaced by residues from a HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147 (1): 86-95 (1991). See also van Dijk and van de Winkel, Curr. Opin. Pharmacol., 5:368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

A “species-dependent antibody” is one which has a stronger binding affinity for an antigen from a first mammalian species than it has for a homologue of that antigen from a second mammalian species. Normally, the species-dependent antibody “binds specifically” to a human antigen (e.g., has a binding affinity (Kd) value of no more than about 1×10⁻⁷ M, preferably no more than about 1×10⁻⁸ M and preferably no more than about 1×10⁻⁹ M) but has a binding affinity for a homologue of the antigen from a second nonhuman mammalian species which is at least about 50 fold, or at least about 500 fold, or at least about 1000 fold, weaker than its binding affinity for the human antigen. The species-dependent antibody can be any of the various types of antibodies as defined above, but preferably is a humanized or human antibody.

The term “hypervariable region,” “HVR,” or “HV,” when used herein refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat |
| | | | | Numbering) |
| H1 | H31-H35 | H26-H35 | H26-H32 | H30-H35 (Chothia |
| | | | | Numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework” or “FR” residues are those variable domain residues other than the HVR residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody.

The expression “linear antibodies” refers to the antibodies described in Zapata et al. (1995 Protein Eng, 8 (10): 1057-1062). Briefly, these antibodies comprise a pair of tandem Fd segments (VH-CH1-VH-CH1) which, together with complementary light chain polypeptides, form a pair of antigen binding regions. Linear antibodies can be bispecific or monospecific.

As use herein, the term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding.

The term “sample,” as used herein, refers to a composition that is obtained or derived from a subject and/or individual of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. Samples include, but are not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof. In some embodiments, the sample is a sample obtained from the cancer of an individual (e.g., a tumor sample) that comprises tumor cells and, optionally, tumor-infiltrating immune cells. For example, the sample can be a tumor specimen that is embedded in a paraffin block, or that includes freshly cut, serial unstained sections. In some embodiments, the sample is from a biopsy and includes 50 or more viable tumor cells (e.g., from a core-needle biopsy and optionally embedded in a paraffin block; excisional, incisional, punch, or forceps biopsy; or a tumor tissue resection).

By “tissue sample”, “tissue specimen” or “cell sample” is meant a collection of similar cells obtained from a tissue, for example a tumor, of a subject or individual. The source of the tissue or cell sample may be solid tissue (e.g., a tumor) as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

A “reference sample”, “reference cell”, “reference tissue”, “control sample”, “control cell”, or “control tissue”, as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject or individual. For example, healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual.

A “reference level”, “reference number”, or “reference frequency”, as used herein, refers to a number, a frequency, an amount, etc. with a predetermined value. In this context, “level” encompasses the absolute number or amount, the relative number or amount, the frequency, the proportion, the percentage, etc. as well as any value or parameter which correlates thereto or can be derived therefrom. As the skilled artisan will appreciate, the reference level, reference number, or reference frequency is predetermined and set to meet routine requirements in terms of e.g., specificity and/or sensitivity. These requirements can vary, e.g., from regulatory body to regulatory body. For example, it may be that assay sensitivity and/or specificity has to be set to certain limits, e.g., 80%, 90%, 95%, 98%, 99%, or 100%. These requirements may also be defined in terms of positive or negative predictive values. Nonetheless, based on the teaching given in the present invention, it will be possible for a skilled artisan to arrive at the reference level, reference number, or reference frequency meeting those requirements. For example, the reference level, reference number, or reference frequency can be determined in reference samples obtained from patients prior to treatment administration or obtained from healthy individuals. The reference level, reference number, or reference frequency in one embodiment has been predetermined in reference samples from the disease entity to which the patient belongs. In certain embodiments, the reference level, reference number, or reference frequency can be statistically calculated or set as determined from the overall distribution of the values in reference samples from a disease entity investigated. In one embodiment the reference level, reference number, or reference frequency is set as a cutoff value as determined from the overall distribution of the values in a disease entity investigated, such that the cutoff value is indicative of the value at which, e.g., the rate of predicting immunogenicity and/or immune response to the therapy described herein reaches a specificity of 100% and a sensitivity of 80%. The reference level, reference number, or reference frequency may vary from patient to patient or may vary depending on various physiological parameters of the patient such as age, gender, or subpopulation, as well as on the number of times treated or vaccinated and the methods used for the determination of (e.g.) the referred to herein. In one embodiment, the reference sample is from essentially the same type of cells, tissue, organ, or body fluid source as the sample from the individual or patient subjected to the method of the invention, e.g., if according to the invention, blood is used as a sample to determine the level of de novo SE TCR clones in the individual, the reference level, reference number, or reference frequency is also determined in blood or a part thereof.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and progression free survival); resulting in an objective response (including a complete response or a partial response); or improving signs or symptoms of cancer.

A patient who “does not have an effective response” to treatment refers to a patient who does not have any one of extending survival (including overall survival and progression free survival); resulting in an objective response (including a complete response or a partial response); or improving signs or symptoms of cancer.

A patient who is “likely to respond” to a therapy refers to a patient who has been identified based on one or more specific biological features or traits relative to the disease, disorder, or condition (such as cancer) that are correlated with treatment responsiveness (or effective response to treatment). This correlation can be identified statistically such that a patient identified as “likely to respond” can refer to a patient with a calculable statistical probability of likelihood to show an effective response to treatment.

The phrase “responsive to” in the context of the present invention indicates that a patient suffering from, being suspected to suffer or being prone to suffer from, or diagnosed with a disorder as described herein, shows a positive response to a treatment, for example an individualized RNA vaccine treatment described herein. Treatment response can be defined based on progression free survival (PFS), overall survival (OS), and overall response rate (ORR), including partial response or complete response to treatment.

The term “individualized” context of the present invention indicates that a therapy or treatment is unique to each patient, for example has been designed for, constructed, or otherwise manufactured based on the specifications, e.g., the genomic profile, immunological profile, metabolic profile, cancer type, cancer antigen profile, somatic mutation profile, age, sex, etc. or the treatment needs of an individual patient. For example, the RNA vaccine of the present disclosure is individualized to each patient such that the individualized RNA vaccine targets one or more neoepitopes resulting from cancer-specific somatic mutations present in, e.g., a pancreatic cancer tumor specimen from each patient that may be unique to each patient.

A “functional Fc region” possesses an “effector function” of a native sequence Fc region. Exemplary “effector functions” include Clq binding; CDC; Fc receptor binding; ADCC; phagocytosis; down regulation of cell surface receptors (e.g., B cell receptor; BCR), etc. Such effector functions generally require the Fc region to be combined with a binding domain (e.g., an antibody variable domain) and can be assessed using various assays as disclosed, for example, in definitions herein.

A cancer or biological sample which “has human effector cells” is one which, in a diagnostic test, has human effector cells present in the sample (e.g., infiltrating human effector cells).

A cancer or biological sample which “has FcR-expressing cells” is one which, in a diagnostic test, has FcR-expressing present in the sample (e.g., infiltrating FcR-expressing cells). In some embodiments, FcR is FcγR. In some embodiments, FcR is an activating FcγR.

The phrase “selecting a patient” or “identifying a patient” as used herein refers to using the information or data generated relating to the number and/or frequency of TCR clones (such as significantly expanded (SE) TCR clones, e.g., de novo SE TCR clones) in a sample of a patient to identify or select the patient as likely to benefit from a therapy comprising an individualized RNA vaccine. The information or data used or generated may be in any form, e.g., written, oral, or electronic. In some embodiments, using the information or data generated includes communicating, presenting, reporting, storing, sending, transferring, supplying, transmitting, dispensing, or combinations thereof. In some embodiments, communicating, presenting, reporting, storing, sending, transferring, supplying, transmitting, dispensing, or combinations thereof are performed by a computing device, analyzer unit or combination thereof. In some further embodiments, communicating, presenting, reporting, storing, sending, transferring, supplying, transmitting, dispensing, or combinations thereof are performed by a laboratory or medical professional. In some embodiments, the information or data includes a comparison of the number and/or frequency of TCR clones (such as significantly expanded (SE) TCR clones, e.g., de novo SE TCR clones) to a reference level. In some embodiments, the information or data includes an indication that TCR clones (such as significantly expanded (SE) TCR clones, e.g., de novo SE TCR clones) are present in the sample. In some embodiments, the information or data includes an indication that the patient is more likely to respond to a therapy comprising an individualized RNA vaccine.

### II. Methods of Treating Pancreatic Cancer

Certain aspects of the present disclosure relate to methods for treating pancreatic cancer in a patient, such as a human patient in need thereof, by administering to the patient an effective amount of an individualized RNA vaccine, a PD-1 axis binding antagonist, and a chemotherapeutic treatment. In some embodiments, the individualized RNA vaccine comprises one or more polynucleotides encoding one or more neoepitopes resulting from cancer-specific somatic mutations present in a pancreatic cancer tumor specimen from the patient, e.g., as described in greater detail below.

Any of the individualized RNA vaccines, PD-1 axis binding antagonists, and chemotherapeutic treatments described herein may be used in the methods of the disclosure.

The RNA vaccine according to the present disclosure is designed to induce neoantigen-and/or tumor-specific immune responses in the patient, such as neoantigen-specific T cell responses, and may augment the magnitude and quality of pre-existing neoantigen-specific T cells. Blocking the PD-L1/PD-1 pathway in the patient, e.g., by administration of a PD-1 axis binding antagonist, in combination with the RNA vaccine may enhance priming and/or reactivation of T-cells, and/or improve the activity of dysfunctional T-cells after tumor antigen exposure, thereby enhancing RNA vaccine-induced immune responses. However, cytotoxic chemotherapies, which are standard-of-care treatments for pancreatic cancer, could have an adverse impact on RNA vaccine-induced immune responses if administered concurrently. Thus, administering the RNA vaccine during a priming phase (e.g., including up to 6 priming RNA vaccine doses, for example 2 or 3 priming RNA vaccine doses) prior to chemotherapy can result in improved neoantigen- and/or tumor-specific immune responses in the patient (see, e.g., Example 2, herein), which may be maximized and/or maintained when followed-up with a boost phase (e.g., including up to 6 booster RNA vaccine doses). RNA vaccine-induced immune responses can be further enhanced by administering the RNA vaccine during the priming and boost phases in combination with a PD-1 axis binding antagonist, which could lead to more robust anti-tumor immune responses and improved clinical efficacy.

Accordingly, in some embodiments, the methods for treating pancreatic cancer provided herein comprise administering an individualized RNA vaccine, a PD-1 axis binding antagonist, and a chemotherapeutic treatment during a treatment period that comprises three phases: a priming phase, a chemotherapy phase after the priming phase, and a boost phase after the chemotherapy phase, wherein the priming phase comprises administering at least one dose of the RNA vaccine (e.g., up to 6 priming RNA vaccine doses, for example 2 or 3 priming RNA vaccine doses) and at least one dose of the PD-1 axis binding antagonist, the chemotherapy phase comprises administering the chemotherapeutic treatment after administration of the RNA vaccine during the priming phase, and the boost phase comprises administering at least one dose of the RNA vaccine (e.g., up to 6 booster RNA vaccine doses) and at least one dose of the PD-1 axis binding antagonist.

In some embodiments, a pancreatic cancer, e.g., a pancreatic cancer tumor, to be treated according to the methods of the present disclosure is an exocrine pancreatic cancer or a neuroendocrine pancreatic cancer. In some embodiments, the pancreatic cancer is an adenocarcinoma (e.g., a pancreatic ductal adenocarcinoma), an acinar cell carcinoma, a squamous cell carcinoma, an adenosquamous carcinoma, a colloid carcinoma, a giant cell tumor, a hepatoid carcinoma, a mucinous cystic neoplasm, an intraductal papillary mucinous neoplasm, a pancreatoblastoma, a serous cystadenoma, a signet ring cell carcinoma, an undifferentiated carcinoma, or solid and pseudopapillary tumors. In some embodiments, the pancreatic cancer is a pancreatic neuroendocrine tumor. In some embodiments, the pancreatic neuroendocrine tumor is an insulinoma, a gastrinoma, a glucagonoma, a VIPoma, a somatostatinoma, or a PPoma. In some embodiments, the pancreatic cancer, e.g., the pancreatic cancer tumor, to be treated according to the methods of the disclosure is a resectable pancreatic cancer, a borderline resectable pancreatic cancer, a locally advanced pancreatic cancer, a metastatic pancreatic cancer, or a recurrent pancreatic cancer. In some embodiments, the pancreatic cancer, e.g., the pancreatic cancer tumor, to be treated according to the methods of the disclosure is resectable. In some embodiments, the pancreatic cancer is a pancreatic ductal adenocarcinoma (PDAC). In some embodiments, the PDAC is resectable.

### (i) Priming Phases

In some embodiments, the methods for treating pancreatic cancer provided herein comprise administering to a patient, such as a human patient in need thereof, an individualized RNA vaccine and a PD-1 axis binding antagonist during a priming phase of treatment.

In some embodiments, the priming phase begins at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 weeks, at least about 14 weeks, or at least about 15 weeks after resection of a pancreatic cancer tumor from the patient, such as a PDAC tumor. In some embodiments, the priming phase begins between about 6 and about 12 weeks after resection of a pancreatic cancer tumor from the patient, such as a PDAC tumor.

In some embodiments, the priming phase comprises any of between about 1 and about 12 weeks, between about 1 and about 8 weeks, or between about 1 and about 6 weeks. In some embodiments, the priming phase comprises 6 weeks.

In some embodiments, the priming phase comprises administering to the patient at least one dose of the RNA vaccine and at least one dose of the PD-1 axis binding antagonist.

In some embodiments, the priming phase comprises administering to the patient any of at least 2, at least 3, at least 4, at least 5, at least 6, at least 7, at least 8, at least 9, at least 10, at least 11, at least 12, or more, doses of the RNA vaccine. In some embodiments, between 1 and 8, between 6 and 8, between 2 and 6, or either 2 or 3 doses of the RNA vaccine are administered to the patient during the priming phase. In some embodiments, 2 doses of the RNA vaccine are administered to the patient during the priming phase. In some embodiments, 3 doses of the RNA vaccine are administered to the patient during the priming phase. In some embodiments, 6 doses of the RNA vaccine are administered to the patient during the priming phase. In some embodiments, 8 doses of the RNA vaccine are administered to the patient during the priming phase. In other embodiments, no more than 6 doses of the RNA vaccine are administered to the patient during the priming phase. In some embodiments, a dose of RNA vaccine may be administered as a single composition, or may be administered in more than one composition. For example, in some cases, an RNA vaccine dose is administered as two separate compositions administered sequentially.

In some embodiments, the priming phase comprises administering the RNA vaccine once per week (QW), once every two weeks (Q2W), once every three weeks (Q3W), once every four weeks (Q4W), once every five weeks (Q5W), once every six weeks (Q6W), once every seven weeks (Q7W), or once every eight weeks (Q8W). In some embodiments, the priming phase comprises administering the RNA vaccine once per week (QW), e.g., once every 7 days. In some embodiments, administration of the RNA vaccine during priming phase begins on day 1 of week 1 of the priming phase. In some embodiments, the priming phase comprises administering the RNA vaccine on day 1 of week 1 of the priming phase and once per week (QW), e.g., once every 7 days, thereafter.

In some embodiments, the priming phase comprises administering six doses of the RNA vaccine. For example, in some cases, the RNA vaccine is administered on day 1 of week 1, day 1 of week 2, day 1 of week 3, day 1 of week 4, day 1 of week 5, and day 1 of week 6 of the priming phase.

In some embodiments, the priming phase comprises administering to the patient one dose of the PD-1 axis binding antagonist. In some embodiments, one dose of the PD-1 axis binding antagonist is administered to the patient during the priming phase. In some embodiments, the dose of the PD-1 axis binding antagonist administered to the patient during the priming phase is administered on the same day as the administration of a dose of the RNA vaccine.

In some embodiments, the priming phase comprises administering to the patient at least two doses of the PD-1 axis binding antagonist. In some embodiments, the priming phase comprises administering to the patient any of at least 2, at least 3, at least 4, at least 5, at least 6, at least 7, at least 8, at least 9, at least 10, at least 11, at least 12, or more, doses of the PD-1 axis binding antagonist. In some embodiments, between 1 and 8, between 6 and 8, between 2 and 6, or either 2 or 3 doses of the PD-1 axis binding antagonist are administered to the patient during the priming phase. In some embodiments, 2 doses of the PD-1 axis binding antagonist are administered to the patient during the priming phase. In some embodiments, any dose of the PD-1 axis binding antagonist administered to the patient during the priming phase is administered on the same day as the administration of a dose of the RNA vaccine.

In some embodiments, the priming phase comprises administering the PD-1 axis binding antagonist once per week (QW), once every two weeks (Q2W), once every three weeks (Q3W), once every four weeks (Q4W), once every five weeks (Q5W), once every six weeks (Q6W), once every seven weeks (Q7W), or once every eight weeks (Q8W). In some embodiments, the priming phase comprises administering the PD-1 axis binding antagonist once every four weeks (Q4W), e.g., once every 28 days. In some embodiments, administration of the PD-1 axis binding antagonist during priming phase begins on day 1 of week 1 of the priming phase. In some embodiments, the priming phase comprises administering the PD-1 axis binding antagonist on day 1 of week 1 of the priming phase and once every four weeks (Q4W), e.g., once every 28 days, thereafter.

In some embodiments, the priming phase comprises administering one dose of the PD-1 axis binding antagonist. For example, in some cases, the PD-1 axis binding antagonist is administered on day 1 of week 3 of the priming phase.

In some embodiments, the priming phase comprises administering the RNA vaccine on day 1 of week 1, day 1 of week 2, day 1 of week 3, day 1 of week 4, day 1 of week 5, and day 1 of week 6 of the priming phase, and the PD-1 axis binding antagonist on day 1 of week 3 of the priming phase.

In some embodiments, the priming phase comprises administering two doses of the PD-1 axis binding antagonist. For example, in some cases, the PD-1 axis binding antagonist is administered on day 1 of week 1, and day 1 of week 5 of the priming phase.

In some embodiments, the priming phase comprises administering the RNA vaccine on day 1 of week 1, day 1 of week 2, day 1 of week 3, day 1 of week 4, day 1 of week 5, and day 1 of week 6 of the priming phase, and the PD-1 axis binding antagonist on day 1 of week 1 and day 1 of week 5 of the priming phase.

Exemplary priming phases are provided in Table 1, below.

TABLE 1
Exemplary priming phases A and B.

| Priming phase week: | | | | | |
|---|---|---|---|---|---|
| Week 1 | Week 2 | Week 3 | Week 4 | Week 5 | Week 6 |

Priming Phase A (1-week or 7-day cycles)

| | | | | | | |
|---|---|---|---|---|---|---|
| Administration | Day 1, | Day 1, | Day 1, | Day 1, | Day 1, | Day 1, |
| of RNA | week 1 | week 2 | week 3 | week 4 | week 5 | week 6 |
| vaccine | | | | | | |
| Administration | Day 1, | — | — | — | Day 1, | — |
| of PD-1 axis | week 1 | | | | week 5 | |
| binding | | | | | | |
| antagonist | | | | | | |

Priming Phase B (1-week or 7-day cycles)

| | | | | | | |
|---|---|---|---|---|---|---|
| Administration | Day 1, | Day 1, | Day 1, | Day 1, | Day 1, | Day 1, |
| of RNA | week 1 | week 2 | week 3 | week 4 | week 5 | week 6 |
| vaccine | | | | | | |
| Administration | — | — | Day 1, | — | — | — |
| of PD-1 axis | | | week 3 | | | |
| binding | | | | | | |
| antagonist | | | | | | |

In some embodiments, the RNA vaccine is administered to the patient during the priming phase at a dose of between about 15 μg to about 50 μg (e.g., any of about 15 μg, about 20 μg, about 25 μg, about 30 μg, about 35 μg, about 38 μg, about 40 μg, about 45 μg, or about 50 μg). In some embodiments, the RNA vaccine is administered to the patient at a dose of about 15 μg, about 21 μg, about 21.3 μg, about 25 μg, about 38 μg, or about 50 μg. In some embodiments, the RNA vaccine is administered to the patient at a dose of 25 μg. In some embodiments, the RNA vaccine is administered to the patient at a dose of about 21 μg. In some embodiments, the RNA vaccine is administered to the patient at a dose of about 21.3 μg. In certain embodiments, the RNA vaccine is administered intravenously to the patient. In some embodiments, a total dose of RNA vaccine may be administered as a single composition, or may be administered in more than one composition. For example, in some cases, the RNA vaccine is administered at a total dose of 25 μg, which is split into two compositions that are administered sequentially. In some embodiments, the RNA vaccine dose is administered to the patient in two equal half-doses. In some embodiments, the two equal half-doses are administered sequentially, optionally with an observation period between the administered equal half-doses. In some embodiments, the dose of about 25 μg is split into two equal half-doses of about 12.5 μg, each administered over 1 minute, optionally with a 5-minute observation period between the administered equal half-doses. In some embodiments, the RNA vaccine comprises one or more polynucleotides encoding 5-20 or 10⁻²⁰ neoepitopes resulting from cancer-specific somatic mutations present in a tumor specimen from the patient. In some embodiments, the one or more polynucleotides of the RNA vaccine are formulated with one or more lipids. In some embodiments, the RNA vaccine is formulated as a lipid nanoparticle, wherein the one or more polynucleotides of the RNA vaccine and one or more lipids form the lipid nanoparticle. In some embodiments, the RNA vaccine is formulated as a lipoplex, wherein the one or more polynucleotides of the RNA vaccine and one or more lipids form the lipoplex.

In some specific embodiments, the PD-1 axis binding antagonist is an anti-PD-L1 antibody, e.g., as described below. In some embodiments, the anti-PD-L1 antibody is avelumab, durvalumab, or atezolizumab. In one embodiment, the anti-PD-L1 antibody is atezolizumab. In some embodiments, the anti-PD-L1 antibody is administered to the patient at a dose of about 1200 mg or about 1680 mg. In some embodiments, the anti-PD-L1 antibody is administered to the patient at a dose of about 1680 mg. In certain embodiments, the PD-1 axis binding antagonist is administered intravenously to the patient.

### (ii) Chemotherapy Phases

In some embodiments, the methods for treating pancreatic cancer provided herein comprise administering to a patient, such as a human patient in need thereof, a chemotherapeutic treatment during a chemotherapy phase following a priming phase, e.g., as described above.

In some embodiments, the chemotherapy phase begins at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 weeks, at least about 14 weeks, or at least about 15 weeks, after the end of the priming phase, such as after the last administration of a dose of the RNA vaccine during the priming phase. In some embodiments, the chemotherapy phase begins between about 1 week and about 9 weeks (e.g., any of 1, 2, 3, 4, 5, 6, 7, 8, or 9 weeks) after the end of the priming phase, such as after the last administration of a dose of the RNA vaccine during the priming phase. In some embodiments, the chemotherapy phase begins on any of week 7, week 8, or week 9, timing starting with week 1 of the priming phase. In some embodiments, the chemotherapy phase begins on week 7, timing starting with week 1 of the priming phase. In some embodiments, the chemotherapy phase begins no later than week 9, timing starting with week 1 of the priming phase. In some embodiments, the priming phase includes six weeks (e.g., as described above), and the chemotherapy phase begins on any of week 7, week 8, or week 9, timing starting with week 1 of the priming phase. In some embodiments, the priming phase includes six weeks (e.g., as described above), and the chemotherapy phase begins no later than week 9, timing starting with week 1 of the priming phase. In some embodiments, the priming phase includes six weeks (e.g., as described above), and the chemotherapy phase begins on week 7, timing starting with week 1 of the priming phase. In some embodiments, the priming phase comprises administering the RNA vaccine on day 1 of week 1, day 1 of week 2, day 1 of week 3, day 1 of week 4, day 1 of week 5, and day 1 of week 6 of the priming phase, the PD-1 axis binding antagonist on day 1 of week 1 and day 1 of week 5 of the priming phase, e.g., as described above, and the chemotherapy phase begins on day 1 of week 7, day 1 of week 8, or day 1 of week 9. In some embodiments, the priming phase comprises administering the RNA vaccine on day 1 of week 1, day 1 of week 2, day 1 of week 3, day 1 of week 4, day 1 of week 5, and day 1 of week 6 of the priming phase, the PD-1 axis binding antagonist on day 1 of week 3 of the priming phase, e.g., as described above, and the chemotherapy phase begins on day 1 of week 7, day 1 of week 8, or day 1 of week 9. In some embodiments, the chemotherapy phase begins on day 1 of week 7.

In some embodiments, the chemotherapy phase comprises administering the chemotherapeutic treatment to the patient once per week (QW), once every two weeks (Q2W), once every three weeks (Q3W), once every four weeks (Q4W), once every five weeks (Q5W), once every six weeks (Q6W), once every seven weeks (Q7W), or once every eight weeks (Q8W). In some embodiments, the chemotherapy phase comprises administering the chemotherapeutic treatment to the patient once every two weeks (Q2W), e.g., once every 14 days. In some embodiments, administration of the chemotherapeutic treatment during the chemotherapy phase begins on day 1 of week 1 of the chemotherapy phase. In some embodiments, the chemotherapy phase comprises administering the chemotherapeutic treatment to the patient on day 1 of week 1 of the chemotherapy phase, and once every two weeks (Q2W), e.g., once every 14 days, thereafter.

In some embodiments, the chemotherapy phase comprises any of at least about 8 weeks, at least about 9 weeks, at least about 10 weeks, at least about 11 weeks, at least about 12 weeks, at least about 13 weeks, at least about 14 weeks, at least about 15 weeks, at least about 16 weeks, at least about 17 weeks, at least about 18 weeks, at least about 19 weeks, at least about 20 weeks, at least about 21 weeks, at least about 22 weeks, at least about 23 weeks, at least about 24 weeks, at least about 25 weeks, at least about 26 weeks, at least about 27 weeks, at least about 28 weeks, at least about 29 weeks, at least about 30 weeks, or more. In some embodiments, the chemotherapy phase comprises about 23 weeks. In some embodiments, the chemotherapy phase comprises about 24 weeks.

In some embodiments, the chemotherapy phase comprises administering to the patient at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, or at least 24, or more, administrations of the chemotherapeutic treatment. In some embodiments, the chemotherapy phase comprises administering to the patient at least 12 administrations of the chemotherapeutic treatment. In some embodiments, the chemotherapy phase comprises administering to the patient 12 administrations of the chemotherapeutic treatment. In some embodiments, the chemotherapy phase comprises administering to the patient the chemotherapeutic treatment starting on day 1 of week 1 of the chemotherapy phase, and every two weeks (Q2W), e.g., every 14 days, thereafter for a total of 12 administration of the chemotherapeutic treatment. For example, in some cases, the chemotherapy phase comprises administering to the patient the chemotherapeutic treatment on day 1 of week 1, day 1 of week 3, day 1 of week 5, day 1 of week 7, day 1 of week 9, day 1 of week 11, day 1 of week 13, day 1 of week 15, day 1 of week 17, day 1 of week 19, day 1 of week 21, and day 1 of week 23 of the chemotherapy phase.

In some embodiments, the chemotherapy phase comprises administering to the patient the chemotherapeutic treatment in 2-week cycles (e.g., 14-day cycles) starting on day 1 of week 1 of the chemotherapy phase. In some embodiments, the chemotherapy phase comprises administering the chemotherapeutic treatment to the patient for at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, or at least 24, or more, 2-week cycles (e.g., 14-day cycles). In some embodiments, the chemotherapy phase comprises at least 12 2-week cycles (e.g., 14-day cycles) of the chemotherapeutic treatment. In some embodiments, the chemotherapy phase comprises 12 2-week cycles (e.g., 14-day cycles) of the chemotherapeutic treatment. In some embodiments, the chemotherapy phase comprises administering to the patient the chemotherapeutic treatment in 2-week cycles (e.g., 14-day cycles) starting on day 1 of cycle 1 of the chemotherapy phase, for a total of 12 cycles of the chemotherapeutic treatment. For example, in some cases, the chemotherapy phase comprises administering to the patient the chemotherapeutic treatment on day 1 of cycle 1, day 1 of cycle 2, day 1 of cycle 3, day 1 of cycle 4, day 1 of cycle 5, day 1 of cycle 6, day 1 of cycle 7, day 1 of cycle 8, day 1 of cycle 9, day 1 of cycle 10, day 1 of cycle 11, and day 1 of cycle 12 of the chemotherapy phase.

In some embodiments, the chemotherapy phase begins on week 7 (e.g., day 1 of week 7), timing starting with week 1 of the priming phase. In some embodiments, the priming phase includes six weeks (e.g., as described above), and the chemotherapy phase begins on week 7 (e.g., day 1 of week 7), timing starting with week 1 of the priming phase. In some embodiments, the chemotherapy phase comprises administering to the patient the chemotherapeutic treatment on day 1 of week 7, day 1 of week 9, day 1 of week 11, day 1 of week 13, day 1 of week 15, day 1 of week 17, day 1 of week 19, day 1 of week 21, day 1 of week 23, day 1 of week 25, day 1 of week 27, and day 1 of week 29, timing starting with week 1 of the priming phase.

An exemplary chemotherapy phase is provided in Table 2, below.

TABLE 2
Exemplary chemotherapy phases.
Chemotherapy (chemo) Phase (2-week or 14-day cycles)

| Chemo | | Chemo | |
|---|---|---|---|
| phase week | | phase week | |
| (starting | | (starting | Administration of |
| with week 1 | Chemo | with week 1 | chemotherapeutic |
| of chemo | phase | of priming | treatment (week: W, |
| phase) | cycle | phase) | cycle: C) |
| Week 1 | Cycle 1 | Week 7 | Day 1 of W 1 (starting with |
| Week 2 | | Week 8 | W 1 of chemo phase); |
| | | | Day 1 of C1; or |
| | | | Day 1 of W 7 (starting with |
| | | | W 1 of priming phase). |
| Week 3 | Cycle 2 | Week 9 | Day 1 of W 3 (starting with |
| Week 4 | | Week 10 | W 1 of chemo phase); |
| | | | Day 1 of C2; or |
| | | | Day 1 of W 9 (starting with |
| | | | W 1 of priming phase). |
| Week 5 | Cycle 3 | Week 11 | Day 1 of W 5 (starting with |
| Week 6 | | Week 12 | W 1 of chemo phase); |
| | | | Day 1 of C3; or |
| | | | Day 1 of W 11 (starting with |
| | | | W 1 of priming phase). |
| Week 7 | Cycle 4 | Week 13 | Day 1 of W 7 (starting with |
| Week 8 | | Week 14 | W 1 of chemo phase); |
| | | | Day 1 of C4; or |
| | | | Day 1 of W 13 (starting with |
| | | | W 1 of priming phase). |
| Week 9 | Cycle 5 | Week 15 | Day 1 of W 9 (starting with |
| | | | W 1 of chemo phase); |
| | | | Day 1 of C5; or |
| Week 10 | | Week 16 | Day 1 of W 15 (starting with |
| | | | W 1 of priming phase). |
| Week 11 | Cycle 6 | Week 17 | Day 1 of W 11 (starting with |
| Week 12 | | Week 18 | W 1 of chemo phase); |
| | | | Day 1 of C6; or |
| | | | Day 1 of W 17 (starting with |
| | | | W 1 of priming phase). |
| Week 13 | Cycle 7 | Week 19 | Day 1 of W 13 (starting with |
| Week 14 | | Week 20 | W 1 of chemo phase); |
| | | | Day 1 of C7; or |
| | | | Day 1 of W 19 (starting with |
| | | | W 1 of priming phase). |
| Week 15 | Cycle 8 | Week 21 | Day 1 of W 15 (starting with |
| Week 16 | | Week 22 | W 1 of chemo phase); |
| | | | Day 1 of C8; or |
| | | | Day 1 of W 21 (starting with |
| | | | W 1 of priming phase). |
| Week 17 | Cycle 9 | Week 23 | Day 1 of W 17 (starting with |
| Week 18 | | Week 24 | W 1 of chemo phase); |
| | | | Day 1 of C9; or |
| | | | Day 1 of W 23 (starting with |
| | | | W 1 of priming phase). |
| Week 19 | Cycle 10 | Week 25 | Day 1 of W 19 (starting with |
| Week 20 | | Week 26 | W 1 of chemo phase); |
| | | | Day 1 of C10; or |
| | | | Day 1 of W 25 (starting with |
| | | | W 1 of priming phase). |
| Week 21 | Cycle 11 | Week 27 | Day 1 of W 21 (starting with |
| Week 22 | | Week 28 | W 1 of chemo phase); |
| | | | Day 1 of C11; or |
| | | | Day 1 of W 27 (starting with |
| | | | W 1 of priming phase). |
| Week 23 | Cycle 12 | Week 29 | Day 1 of W 23 (starting with |
| Week 24 | | Week 30 | W 1 of chemo phase); |
| | | | Day 1 of C12; or |
| | | | Day 1 of W 29 (starting with |
| | | | W 1 of priming phase). |

In some embodiments, the chemotherapeutic treatment administered during the chemotherapy phase is any chemotherapy known in the art or described herein, such as a chemotherapy for pancreatic cancer, and may be administered during the chemotherapy phase according to any of the chemotherapy phase administration regimens described herein, or according to standard dosing regimens known in the art for such a chemotherapy.

In some specific embodiments, the chemotherapeutic treatment comprises one or more of gemcitabine, leucovorin, 5-fluorouracil, capecitabine, irinotecan, liposomal irinotecan, a platinum-based chemotherapeutic agent, a taxane, and any combination thereof. In some embodiments, the chemotherapeutic treatment comprises leucovorin (e.g., leucovorin calcium, folinic acid or calcium folinate), 5-fluorouracil (e.g., fluorouracil), irinotecan (e.g., irinotecan hydrochloride), and oxaliplatin. In some specific embodiments, the chemotherapeutic treatment is a FOLFIRINOX treatment. FOLFIRINOX is a combination chemotherapy treatment that includes a combination of leucovorin (e.g., leucovorin calcium, folinic acid or calcium folinate), 5-fluorouracil (e.g., fluorouracil), irinotecan (e.g., irinotecan hydrochloride), and oxaliplatin. In some embodiments, the FOLFIRINOX chemotherapeutic treatment comprises administering to the patient oxaliplatin at a dose of 85 mg/m², leucovorin at a dose of 400 mg/m², irinotecan at a dose of 180 mg/m², 5-fluorouracil bolus at a dose of 400 mg/m², and 5-fluorouracil at a dose of 2400 mg/m² (e.g., administered as an intravenous infusion over about 46 hours). In some embodiments, the chemotherapeutic treatment is a modified FOLFIRINOX treatment (mFOLFIRINOX). mFOLFIRINOX is a combination chemotherapy treatment that includes a combination of leucovorin (e.g., leucovorin calcium, folinic acid or calcium folinate), 5-fluorouracil (e.g., fluorouracil), irinotecan (e.g., irinotecan hydrochloride), and oxaliplatin, but is modified with respect to FOLFIRINOX, e.g., to reduce the dose of one or more of the individual agents in the combination and/or removal of the 5-fluorouracil bolus. For example, in some cases, the mFOLFIRINOX chemotherapy comprises administering to the patient oxaliplatin at a dose of about 85 mg/m², leucovorin at a dose of about 400 mg/m², irinotecan at a dose of about 150 mg/m², and/or 5-fluorouracil at a dose of about 2400 mg/m² (e.g., administered as an intravenous infusion over about 46 hours). In some embodiments, the chemotherapy phase comprises administering to the patient oxaliplatin at a dose of about 85 mg/m², leucovorin at a dose of about 400 mg/m², irinotecan at a dose of about 150 mg/m², and 5-fluorouracil at a dose of about 2400 mg/m². In some specific embodiments, the chemotherapy phase comprises administering to the patient oxaliplatin at a dose of 85 mg/m² intravenously over about 2 hours (e.g., ±5 minutes), leucovorin at a dose of 400 mg/m² intravenously over about 2 hours (e.g., ±15 minutes), irinotecan at a dose of 150 mg/m² intravenously over about 90 minutes (e.g., ±5 minutes), for example beginning about 30 minutes after leucovorin infusion is started, and 5-fluorouracil at a dose of 2400 mg/m² intravenously as a continuous infusion over about 46 hours (e.g., ±2 hours).

(iii) Boost Phases

In some embodiments, the methods for treating pancreatic cancer provided herein comprise administering to a patient, such as a human patient in need thereof, at least one dose of an RNA vaccine and at least one dose of a PD-1 axis binding antagonist during a boost phase after the end of a chemotherapy phase, e.g., as described above.

In some embodiments, the boost phase begins at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 weeks, at least about 14 weeks, or at least about 15 weeks after the end of the chemotherapy phase, e.g., after the last administration of chemotherapeutic treatment. In some embodiments, the boost phase begins between about 3 weeks and about 12 weeks, or between about 4 weeks and about 12 weeks after the end of the chemotherapy phase, e.g., after the last administration of chemotherapeutic treatment. In some embodiments, the boost phase begins 3 weeks after the end of the chemotherapy phase, e.g., after the last administration of chemotherapeutic treatment. In some embodiments, the boost phase begins 4 weeks after the end of the chemotherapy phase, e.g., after the last administration of chemotherapeutic treatment. In some embodiments, the boost phase begins no later than 12 weeks after the end of the chemotherapy phase, e.g., after the last administration of chemotherapeutic treatment.

In some embodiments, the boost phase begins on week 27 (e.g., day 1 of week 27), timing starting with week 1 of the chemotherapy phase. In some embodiments, the chemotherapy phase comprises administering to the patient the chemotherapeutic treatment on day 1 of week 1, day 1 of week 3, day 1 of week 5, day 1 of week 7, day 1 of week 9, day 1 of week 11, day 1 of week 13, day 1 of week 15, day 1 of week 17, day 1 of week 19, day 1 of week 21, and day 1 of week 23 of the chemotherapy phase, e.g., as described above, and the boost phase begins on week 27 (e.g., day 1 of week 27).

In other embodiments, the boost phase begins on week 33 (e.g., day 1 of week 33), timing starting with week 1 of the priming phase. In some embodiments, the chemotherapy phase comprises administering to the patient the chemotherapeutic treatment on day 1 of week 7, day 1 of week 9, day 1 of week 11, day 1 of week 13, day 1 of week 15, day 1 of week 17, day 1 of week 19, day 1 of week 21, day 1 of week 23, day 1 of week 25, day 1 of week 27, and day 1 of week 29, timing starting with week 1 of the priming phase, e.g., as described above, and the boost phase begins on week 33 (e.g., day 1 of week 33).

In some embodiments, the boost phase comprises administering the RNA vaccine to the patient once per week (QW), once every two weeks (Q2W), once every three weeks (Q3W), once every four weeks (Q4W), once every five weeks (Q5W), once every six weeks (Q6W), once every seven weeks (Q7W), or once every eight weeks (Q8W). In some embodiments, the boost phase comprises administering the RNA vaccine to the patient once every four weeks (Q4W), e.g., once every 28 days. In some embodiments, the boost phase comprises administering the PD-1 axis binding antagonist to the patient once per week (QW), once every two weeks (Q2W), once every three weeks (Q3W), once every four weeks (Q4W), once every five weeks (Q5W), once every six weeks (Q6W), once every seven weeks (Q7W), or once every eight weeks (Q8W). In some embodiments, the boost phase comprises administering the PD-1 axis binding antagonist to the patient once every four weeks (Q4W), e.g., once every 28 days. In some embodiments, administrations of the RNA vaccine and the PD-1 axis binding antagonist during the boost phase occur on the same day. In some embodiments, the boost phase comprises administering the RNA vaccine and the PD-1 axis binding antagonist to the patient once per week (QW), once every two weeks (Q2W), once every three weeks (Q3W), once every four weeks (Q4W), once every five weeks (Q5W), once every six weeks (Q6W), once every seven weeks (Q7W), or once every eight weeks (Q8W). In some embodiments, the boost phase comprises administering the RNA vaccine and the PD-1 axis binding antagonist to the patient once every four weeks (Q4W), e.g., once every 28 days.

In some embodiments, administration of the RNA vaccine and/or the PD-1 axis binding antagonist during the boost phase begins on day 1 of week 1 of the boost phase. In some embodiments, the boost phase comprises administering the RNA vaccine and/or the PD-1 axis binding antagonist to the patient on day 1 of the boost phase, and once every four weeks (Q4W), e.g., once every 28 days, thereafter. In some embodiments, administration of the RNA vaccine and the PD-1 axis binding antagonist during the boost phase begins on day 1 of week 1 of the boost phase. In some embodiments, the boost phase comprises administering the RNA vaccine and the PD-1 axis binding antagonist to the patient on day 1 of the boost phase, and once every four weeks (Q4W), e.g., once every 28 days, thereafter.

In some embodiments, the boost phase comprises administering to the patient at least 2, at least 3, at least 4, at least 5, at least 6, at least 7, at least 8, at least 9, at least 10, at least 11, at least 12, or more, doses of the RNA vaccine. In some embodiments, between 1 and 8, between 6 and 8, between 2 and 6, or either 2 or 3 doses of the RNA vaccine are administered to the patient during the boost phase. In some embodiments, 2 doses of the RNA vaccine are administered to the patient during the boost phase. In some embodiments, 3 doses of the RNA vaccine are administered to the patient during the boost phase. In some embodiments, 6 doses of the RNA vaccine are administered to the patient during the boost phase. In some embodiments, 8 doses of the RNA vaccine are administered to the patient during the boost phase. In other embodiments, at least 6 doses of the RNA vaccine are administered to the patient during the boost phase. In other embodiments, no more than 6 doses of the RNA vaccine are administered to the patient during the boost phase. In some embodiments, a dose of RNA vaccine may be administered as a single composition, or may be administered in more than one composition. For example, in some cases, an RNA vaccine dose is administered as two separate compositions administered sequentially. In some embodiments, the boost phase comprises administering to the patient at least 2, at least 3, at least 4, at least 5, at least 6, at least 7, at least 8, at least 9, at least 10, at least 11, at least 12, or more, doses of the PD-1 axis binding antagonist. In some embodiments, between 1 and 8, between 6 and 8, between 2 and 6, or either 2 or 3 doses of the PD-1 axis binding antagonist are administered to the patient during the boost phase. In some embodiments, 2 doses of the PD-1 axis binding antagonist are administered to the patient during the boost phase. In some embodiments, 3 doses of the PD-1 axis binding antagonist are administered to the patient during the boost phase. In some embodiments, 6 doses of the PD-1 axis binding antagonist are administered to the patient during the boost phase. In some embodiments, 8 doses of the PD-1 axis binding antagonist are administered to the patient during the boost phase. In other embodiments, at least 6 doses of the PD-1 axis binding antagonist are administered to the patient during the boost phase. In other embodiments, no more than 6 doses of the PD-1 axis binding antagonist are administered to the patient during the boost phase.

In some embodiments, the boost phase comprises administering to the patient the RNA vaccine starting on day 1 of week 1 of the boost phase, and every four weeks (Q4W), e.g., every 28 days, for example, for a total of 6 administrations of the RNA vaccine. For example, in some cases, the boost phase comprises administering to the patient the RNA vaccine on day 1 of week 1, day 1 of week 5, day 1 of week 9, day 1 of week 13, day 1 of week 17, and day 1 of week 21 of the boost phase. In some embodiments, the boost phase comprises administering to the patient the PD-1 axis binding antagonist starting on day 1 of week 1 of the boost phase, and every four weeks (Q4W), e.g., every 28 days, for example, for a total of 6 administrations of the PD-1 axis binding antagonist. For example, in some cases, the boost phase comprises administering to the patient the PD-1 axis binding antagonist on day 1 of week 1, day 1 of week 5, day 1 of week 9, day 1 of week 13, day 1 of week 17, and day 1 of week 21 of the boost phase. In some embodiments, the boost phase comprises administering to the patient the RNA vaccine and the PD-1 axis binding antagonist starting on day 1 of week 1 of the boost phase, and every four weeks (Q4W), e.g., every 28 days, for example, for a total of 6 administrations of the RNA vaccine and the PD-1 axis binding antagonist. For example, in some cases, the boost phase comprises administering to the patient the RNA vaccine and the PD-1 axis binding antagonist on day 1 of week 1, day 1 of week 5, day 1 of week 9, day 1 of week 13, day 1 of week 17, and day 1 of week 21 of the boost phase. In some embodiments, the boost phase comprises 21 weeks.

In some embodiments, the boost phase comprises administering to the patient the RNA vaccine and/or the PD-1 axis binding antagonist in 4-week cycles (e.g., 28-day cycles), for example, starting on day 1 of week 1 of the boost phase. In some embodiments, the boost phase comprises administering the RNA vaccine to the patient for at least 1, at least 2, at least 3, at least 4, at least 5, at least 6, at least 7, at least 8, at least 9, at least 10, at least 11, at least 12, or more, 4-week cycles (e.g., 28-day cycles). In some embodiments, the RNA vaccine is administered in 4-week cycles (e.g., 28-day cycles) for between 1 and 8, between 6 and 8, between 2 and 6, or either 2 or 3 cycles during the boost base. In some embodiments, the RNA vaccine is administered in 4-week cycles (e.g., 28-day cycles) for 2 cycles during the boost phase. In some embodiments, the RNA vaccine is administered in 4-week cycles (e.g., 28-day cycles) for 3 cycles during the boost phase. In some embodiments, the RNA vaccine is administered in 4-week cycles (e.g., 28-day cycles) for 6 cycles during the boost phase. In some embodiments, the RNA vaccine is administered in 4-week cycles (e.g., 28-day cycles) for 8 cycles during the boost phase. In other embodiments, the RNA vaccine is administered in 4-week cycles (e.g., 28-day cycles) for at least 6 cycles during the boost phase. In other embodiments, the RNA vaccine is administered in 4-week cycles (e.g., 28-day cycles) for no more than 6 cycles during the boost phase.

In some embodiments, the boost phase comprises administering the PD-1 axis binding antagonist to the patient for at least 1, at least 2, at least 3, at least 4, at least 5, at least 6, at least 7, at least 8, at least 9, at least 10, at least 11, at least 12, or more, 4-week cycles (e.g., 28-day cycles). In some embodiments, the PD-1 axis binding antagonist is administered in 4-week cycles (e.g., 28-day cycles) for between 1 and 8, between 6 and 8, between 2 and 6, or either 2 or 3 cycles during the boost base. In some embodiments, the PD-1 axis binding antagonist is administered in 4-week cycles (e.g., 28-day cycles) for 2 cycles during the boost phase. In some embodiments, the PD-1 axis binding antagonist is administered in 4-week cycles (e.g., 28-day cycles) for 3 cycles during the boost phase. In some embodiments, the PD-1 axis binding antagonist is administered in 4-week cycles (e.g., 28-day cycles) for 6 cycles during the boost phase. In some embodiments, the PD-1 axis binding antagonist is administered in 4-week cycles (e.g., 28-day cycles) for 8 cycles during the boost phase. In other embodiments, the PD-1 axis binding antagonist is administered in 4-week cycles (e.g., 28-day cycles) for at least 6 cycles during the boost phase. In other embodiments, the PD-1 axis binding antagonist is administered in 4-week cycles (e.g., 28-day cycles) for no more than 6 cycles during the boost phase.

In some embodiments, the boost phase comprises administering to the patient the RNA vaccine in 4-week cycles (e.g., 28-day cycles) starting on day 1 of week 1 of the boost phase, e.g., for a total of 6 cycles. For example, in some cases, the boost phase comprises administering to the patient the RNA vaccine on day 1 of cycle 1, day 1 of cycle 2, day 1 of cycle 3, day 1 of cycle 4, day 1 of cycle 5, and day 1 of cycle 6 of the boost phase.

In some embodiments, the boost phase comprises administering to the patient the PD-1 axis binding antagonist in 4-week cycles (e.g., 28-day cycles) starting on day 1 of week 1 of the boost phase, e.g., for a total of 6 cycles. For example, in some cases, the boost phase comprises administering to the patient the PD-1 axis binding antagonist on day 1 of cycle 1, day 1 of cycle 2, day 1 of cycle 3, day 1 of cycle 4, day 1 of cycle 5, and day 1 of cycle 6 of the boost phase.

In some embodiments, the boost phase comprises administering to the patient the RNA vaccine and the PD-1 axis binding antagonist in 4-week cycles (e.g., 28-day cycles) starting on day 1 of week 1 of the boost phase, e.g., for a total of 6 cycles. For example, in some cases, the boost phase comprises administering to the patient the RNA vaccine and the PD-1 axis binding antagonist on day 1 of cycle 1, day 1 of cycle 2, day 1 of cycle 3, day 1 of cycle 4, day 1 of cycle 5, and day 1 of cycle 6 of the boost phase.

In some embodiments, the boost phase begins on week 33 (e.g., day 1 of week 33), timing starting with week 1 of the priming phase, e.g., as described above. In some embodiments, the boost phase comprises administering to the patient the RNA vaccine starting on day 1 of week 33, timing starting with week 1 of the priming phase, e.g., as described above, and every four weeks (Q4W; e.g., every 28 days) thereafter, for example, for a total of 6 administration of the RNA vaccine. For example, in some cases, the boost phase comprises administering to the patient the RNA vaccine on day 1 of week 33, day 1 of week 37, day 1 of week 41, day 1 of week 45, day 1 of week 49, and day 1 of week 53 of the boost phase, timing starting with week 1 of the priming phase. In some embodiments, the boost phase comprises administering to the patient the PD-1 axis binding antagonist starting on day 1 of week 33, timing starting with week 1 of the priming phase, e.g., as described above, and every four weeks (Q4W; e.g., every 28 days) thereafter, for example, for a total of 6 administration of the PD-1 axis binding antagonist. For example, in some cases, the boost phase comprises administering to the patient the PD-1 axis binding antagonist on day 1 of week 33, day 1 of week 37, day 1 of week 41, day 1 of week 45, day 1 of week 49, and day 1 of week 53 of the boost phase, timing starting with week 1 of the priming phase. In some embodiments, the boost phase comprises administering to the patient the RNA vaccine and the PD-1 axis binding antagonist starting on day 1 of week 33, timing starting with week 1 of the priming phase, e.g., as described above, and every four weeks (Q4W; e.g., every 28 days) thereafter, for example, for a total of 6 administration of the RNA vaccine and the PD-1 axis binding antagonist. For example, in some cases, the boost phase comprises administering to the patient the RNA vaccine and the PD-1 axis binding antagonist on day 1 of week 33, day 1 of week 37, day 1 of week 41, day 1 of week 45, day 1 of week 49, and day 1 of week 53 of the boost phase, timing starting with week 1 of the priming phase.

An exemplary boost phase is provided in Table 3, below.

TABLE 3
Exemplary boost phases.
Boost Phase (4-week or 28-day cycles)

| Boost | | Boost | |
|---|---|---|---|
| phase week | | phase week | |
| (starting | | (starting | |
| with week 1 | Boost | with week 1 | Administration of RNA vaccine and |
| of boost | phase | of priming | PD-1 axis binding antagonist |
| phase) | cycle | phase) | (week: W, cycle: C) |
| Week 1 | Cycle | Week 33 | Day 1 of W 1 (starting with week 1 |
| Week 2 | 1 | Week 34 | of boost phase); |
| Week 3 | | Week 35 | Day 1 of C1; or |
| Week 4 | | Week 36 | Day 1 of W 33 (starting with week 1 |
| | | | of priming phase). |
| Week 5 | Cycle | Week 37 | Day 1 of W 5 (starting with week 1 |
| Week 6 | 2 | Week 38 | of boost phase); |
| Week 7 | | Week 39 | Day 1 of C2; or |
| Week 8 | | Week 40 | Day 1 of W 37 (starting with week 1 |
| | | | of priming phase). |
| Week 9 | Cycle | Week 41 | Day 1 of W 9 (starting with week 1 |
| Week 10 | 3 | Week 42 | of boost phase); |
| Week 11 | | Week 43 | Day 1 of C3; or |
| Week 12 | | Week 44 | Day 1 of W 41 (starting with week 1 |
| | | | of priming phase). |
| Week 13 | Cycle | Week 45 | Day 1 of W 13 (starting with week 1 |
| Week 14 | 4 | Week 46 | of boost phase); |
| Week 15 | | Week 47 | Day 1 of C4; or |
| Week 16 | | Week 48 | Day 1 of W 45 (starting with week 1 |
| | | | of priming phase). |
| Week 17 | Cycle | Week 49 | Day 1 of W 17 (starting with week 1 |
| Week 18 | 5 | Week 50 | of boost phase); |
| Week 19 | | Week 51 | Day 1 of C5; or |
| Week 20 | | Week 52 | Day 1 of W 49 (starting with week 1 |
| | | | of priming phase). |
| Week 21 | Cycle | Week 53 | Day 1 of W 21 (starting with week 1 |
| Week 22 | 6 | Week 54 | of boost phase); |
| Week 23 | | Week 55 | Day 1 of C6; or |
| Week 24 | | Week 56 | Day 1 of W 53 (starting with week 1 |
| | | | of priming phase). |

In some embodiments, the RNA vaccine is administered to the patient during the boost phase at a dose of between about 15 μg to about 50 μg (e.g., any of about 15 μg, about 20 μg, about 25 μg, about 30 μg, about 35 μg, about 38 μg, about 40 μg, about 45 μg, or about 50 μg). In some embodiments, the RNA vaccine is administered to the patient at a dose of about 15 μg, about 21 μg, about 21.3 μg, about 25 μg, about 38 μg, or about 50 μg. In some embodiments, the RNA vaccine is administered to the patient at a dose of 25 μg. In some embodiments, the RNA vaccine is administered to the patient at a dose of about 21 μg. In some embodiments, the RNA vaccine is administered to the patient at a dose of about 21.3 μg. In certain embodiments, the RNA vaccine is administered intravenously to the patient. In some embodiments, a total dose of RNA vaccine may be administered as a single composition, or may be administered in more than one composition. For example, in some cases, the RNA vaccine is administered at a total dose of 25 μg, which is split into two compositions that are administered sequentially. In some embodiments, the RNA vaccine dose is administered to the patient in two equal half-doses. In some embodiments, the two equal half-doses are administered sequentially, optionally with an observation period between the administered equal half-doses. In some embodiments, the dose of about 25 μg is split into two equal half-doses of about 12.5 μg, each administered over 1 minute, optionally with a 5-minute observation period between the administered equal half-doses. In some embodiments, the RNA vaccine comprises one or more polynucleotides encoding 5-20 or 10⁻²⁰ neoepitopes resulting from cancer-specific somatic mutations present in a tumor specimen from the patient. In some embodiments, the one or more polynucleotides of the RNA vaccine are formulated with one or more lipids. In some embodiments, the RNA vaccine is formulated as a lipid nanoparticle, wherein the one or more polynucleotides of the RNA vaccine and one or more lipids form the lipid nanoparticle. In some embodiments, the RNA vaccine is formulated as a lipoplex, wherein the one or more polynucleotides of the RNA vaccine and one or more lipids form the lipoplex.

In some specific embodiments, the PD-1 axis binding antagonist is an anti-PD-L1 antibody, e.g., as described below. In some embodiments, the anti-PD-L1 antibody is avelumab, durvalumab, or atezolizumab. In one embodiment, the anti-PD-L1 antibody is atezolizumab. In some embodiments, the anti-PD-L1 antibody is administered to the patient at a dose of about 1200 mg or about 1680 mg. In some embodiments, the anti-PD-L1 antibody is administered to the patient at a dose of about 1680 mg. In certain embodiments, the PD-1 axis binding antagonist is administered intravenously to the patient.

Any of the priming phases, chemotherapy phases, and boost phases described herein may be used in any combination in the methods for treating pancreatic cancer provided herein.

For example, in some embodiments, the methods of treating pancreatic cancer provided herein comprise administering to the patient an individualized RNA vaccine, a PD-1 axis binding antagonist, and a chemotherapeutic treatment during a treatment period that comprises a priming phase, a chemotherapy phase after the priming phase, and a boost phase after the chemotherapy phase, wherein: the priming phase comprises administering the RNA vaccine on day 1 of weeks 1, 2, 3, 4, 5, and 6 of the priming phase, and the PD-1 axis binding antagonist on day 1 of week 3 of the priming phase; the chemotherapy phase comprises administering a chemotherapeutic treatment on day 1 of weeks 7, 9, 11, 13, 15, 17, 19, 21, 23, 25, 27, and 29, timing starting with week 1 of the priming phase; and the boost phase comprises administering the RNA vaccine and the PD-1 axis binding antagonist on day 1 of weeks 33, 37, 41, 45, 49, and 53, timing starting with week 1 of the priming phase. In another example, the methods of treating pancreatic cancer provided herein comprise administering to the patient an individualized RNA vaccine, a PD-1 axis binding antagonist, and a chemotherapeutic treatment during a treatment period that comprises a priming phase, a chemotherapy phase after the priming phase, and a boost phase after the chemotherapy phase, wherein: the priming phase comprises administering the RNA vaccine on day 1 of weeks 1, 2, 3, 4, 5, and 6 of the priming phase, and the PD-1 axis binding antagonist on day 1 of weeks 1 and 5 of the priming phase; the chemotherapy phase comprises administering a chemotherapeutic treatment on day 1 of weeks 7, 9, 11, 13, 15, 17, 19, 21, 23, 25, 27, and 29, timing starting with week 1 of the priming phase; and the boost phase comprises administering the RNA vaccine and the PD-1 axis binding antagonist on day 1 of weeks 33, 37, 41, 45, 49, and 53, timing starting with week 1 of the priming phase. In some embodiments, the priming phase begins at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 weeks, at least about 14 weeks, or at least about 15 weeks after resection of a pancreatic cancer tumor from the patient, such as a pancreatic ductal adenocarcinoma (PDAC) tumor. In some embodiments, the priming phase begins between about 6 and about 12 weeks after resection of a pancreatic cancer tumor from the patient, such as a pancreatic ductal adenocarcinoma (PDAC) tumor.

Individuals with a Tumor

In some embodiments, the pancreatic cancer tumor is a resectable PDAC tumor, which is assessed by preoperative imaging in the human patient with computed tomography (CT) scan with contrast or magnetic resonance imaging (MRI) prior to administration of the RNA vaccine, the PD-1 axis binding antagonist, and the chemotherapeutic treatment. In some embodiments, CT scan with contrast may be contraindicated. In some embodiments wherein CT scan with contrast is contraindicated, CT scan without contrast is used with MRI scan prior to administration of the RNA vaccine, the PD-1 axis binding antagonist, and the chemotherapeutic treatment. In some embodiments, other imaging techniques are performed if clinically indicated. Other imaging techniques may include, but are not limited to, positron emission tomography (PET), ultrasound, 3D ultrasound, radiography, etc. Contrast agents can include, for example, gadolinium, iron oxide, manganese (II), iodine (e.g., Iohexol), barium-sulfate etc. In some embodiments, the pancreatic cancer tumor is a resectable PDAC tumor comprising one or more characteristics selected from the group consisting of: clear fat plane around the celiac and superior mesenteric arteries; patent superior mesenteric and portal veins; no encasement of the superior mesenteric vein or portal veins; no encasement of the superior mesenteric or hepatic arteries; absence of metastatic disease; and absence of extra-regional nodal disease.

In some embodiments, the human patient has a histologically confirmed diagnosis of PDAC prior to administration of the RNA vaccine, the PD-1 axis binding antagonist, and the chemotherapeutic treatment. In some embodiments, the human patient has adenosquamous carcinoma of the pancreas prior to administration of the RNA vaccine, the PD-1 axis binding antagonist, and the chemotherapeutic treatment. In some embodiments, the human patient does not have intraductal papillary mucinous neoplasm-associated PDAC prior to administration of the RNA vaccine, the PD-1 axis binding antagonist, and the chemotherapeutic treatment. In some embodiments, the human patient does not have a pancreatic endocrine tumor or acinar cell adenocarcinoma, pancreatic cystadenocarcinoma, or pancreatic malignant ampulloma prior to administration of the RNA vaccine, the PD-1 axis binding antagonist, and the chemotherapeutic treatment.

In some embodiments, the pancreatic cancer tumor has tumor, lymph node, metastasis (TNM) pathological staging values of T1-T3, NO-N2, or MO prior to administration of the RNA vaccine, the PD-1 axis binding antagonist, and the chemotherapeutic treatment. In some embodiments, the staging values are assessed as per the American Joint Committee on Cancer (AJCC) Cancer Staging Manual, 8th edition (Amin, M. B. et al., eds., American Joint Committee on Cancer (AJCC) Cancer Staging Manual; 8th ed. New York: Springer 2017).

In some embodiments, the pancreatic cancer tumor is a resectable PDAC tumor, wherein: the human patient had no evidence of PDAC disease after resection of the PDAC tumor, and/or the human patient had a macroscopically complete resection of the PDAC tumor prior to administration of the RNA vaccine, the PD-1 axis binding antagonist, and the chemotherapeutic treatment. In some embodiments, the human patient further had an RO or RI resection of the PDAC tumor. In some embodiments, the human patient had unequivocal absence of PDAC after resection of the PDAC tumor. In some embodiments, the absence of PDAC is assessed by CT or MRI scans, one or more biochemical assays, and/or clinical findings. In some embodiments, the one of more biochemical assays include, but are not limited to, carcinoembryonic antigen (CEA) and CA19-9 assays.

In some embodiments, the pancreatic cancer tumor is a resectable PDAC tumor, wherein following resection of the tumor, the human patient did not have unresolved >Grade 3 postoperative complications prior to administration of the RNA vaccine, the PD-1 axis binding antagonist, and the chemotherapeutic treatment. In some embodiments, the complications are assessed according to the Clavien-Dindo Classification of Surgical Complications. The Clavien-Dindo Classification of Surgical Complications identifies five stages: grade 1, wherein any deviation from the normal postoperative course does not require the need for pharmacological treatment or surgical, endoscopic, and radiological interventions; grade 2, wherein pharmacological treatment with drugs may be required; grade 3, wherein may be required surgical, endoscopic, and radiological intervention with or without general anesthesia; grade 4, wherein life-threatening complications occur that may require IC/ICU management and may include single or multiple organ dysfunction; and grade 5, wherein the patient dies.

In some embodiments, the human patient has a CA19-9 level of 180 U/mL or greater prior to administration of the RNA vaccine, the PD-1 axis binding antagonist, and the chemotherapeutic treatment. In some embodiments, the human patient has a CA19-9 level of less than 180 U/mL prior to administration of the RNA vaccine, the PD-1 axis binding antagonist, and the chemotherapeutic treatment. CA19-9, also known as carbohydrate antigen 19-9 or cancer antigen 19-9, is a pancreatic cancer antigen that may be present in blood samples taken from a human pancreatic cancer patient or a human suspected of having pancreatic cancer.

In some embodiments, at least five neoepitopes resulting from cancer-specific somatic mutations are present in the tumor specimen obtained from the human patient prior to administration of the RNA vaccine, the PD-1 axis binding antagonist, and the chemotherapeutic treatment.

In some embodiments, the human patient has an Eastern Cooperative Oncology Group (ECOG) Performance Status of 0 or 1 prior to administration of the RNA vaccine, the PD-1 axis binding antagonist, and the chemotherapeutic treatment. ECOG Performance Status evaluates a patient's ability to care for themself, their daily activity, and their physical ability (for example, walking, working, etc.).

In some embodiments, the human patient has not received an adjuvant, neoadjuvant, or induction treatment for pancreatic cancer, or a systemic anti-cancer treatment for pancreatic cancer, prior to administration of the RNA vaccine, the PD-1 axis binding antagonist, and the chemotherapeutic treatment. In some embodiments, the pancreatic cancer is PDAC.

In some embodiments, the human patient has not had a cytotoxic chemotherapy, immunotherapy, investigational therapy, or radiation therapy prior to administration of the RNA vaccine, the PD-1 axis binding antagonist, and the chemotherapeutic treatment.

In some embodiments, the human patient has a spleen prior to administration of the RNA vaccine, the PD-1 axis binding antagonist, and the chemotherapeutic treatment. In some embodiments, the human patient has not had loss of spleen due to splenectomy, splenic injury/infarction, or functional asplenia prior to administration of the RNA vaccine, the PD-1 axis binding antagonist, and the chemotherapeutic treatment. In some embodiments, the human patient has not had a distal pancreatectomy with splenectomy prior to administration of the RNA vaccine, the PD-1 axis binding antagonist, and the chemotherapeutic treatment.

In some embodiments, the human patient does not have preexisting neuropathy prior to administration of the RNA vaccine, the PD-1 axis binding antagonist, and the chemotherapeutic treatment.

In some embodiments, the human patient does not have an aUGT1A1 genotype associated with poor metabolizer phenotype prior to administration of the RNA vaccine, the PD-1 axis binding antagonist, and the chemotherapeutic treatment. The UGTIAl gene codes for a UDP-glucuronosyltransferase enzyme that aids in the metabolism of drugs such as, for example, irinotecan (SN-38), acetaminophen (paracetamol), carvedilol, etoposide, lamotrigine and simvastatin. Human patients presenting with an aUGTIAl genotype may be at increased risk for irinotecan toxicity following chemotherapy treatment with mFOLFIRINOX (see, e.g., Correia Marques, S. and Ikediobi, O.N. (2010), Hum Genomics; 4 (4): 238-249).

In some embodiments, the human patient does not have an autoimmune disease, immune deficiency, or primary immunodeficiency prior to administration of the RNA vaccine, the PD-1 axis binding antagonist, and the chemotherapeutic treatment. In some embodiments, the human patient has not been treated with: monoamine oxidase inhibitors (MAOIs) within 3 weeks, a systemic immunostimulatory agent within 4 weeks or 5 drug-elimination half-lives, whichever is longer, or a systemic immunosuppressive medication within 2 weeks, prior to administration of the RNA vaccine, the PD-1 axis binding antagonist, and the chemotherapeutic treatment. In some embodiments, the human patient has not had an allogeneic stem cell or solid organ transplantation prior to administration of the RNA vaccine, the PD-1 axis binding antagonist, and the chemotherapeutic treatment.

### Response to Administration

In some embodiments, the method described herein further comprises assessing disease-free survival (DFS) of the human patient after treatment with the RNA vaccine, the PD-1 axis binding antagonist, and the chemotherapeutic treatment. DFS is measured as the time from patient randomization to the first of either the first recurrence of PDAC or first occurrence of new cancer, as determined by the investigator, or death from any cause. In some embodiments, new cancers do not include malignancies that have a negligible risk of metastasis or death (e.g., 5-year OS rate >90%), including but not limited to adequately treated carcinoma in situ of the cervix, non-melanoma skin cancer, localized prostate cancer, ductal carcinoma in situ, or Stage I uterine cancer. In some embodiments, administration of the RNA vaccine, the PD-1 axis binding antagonist, and the chemotherapeutic treatment results in an improvement in DFS of the human patient as compared to DFS of a corresponding human patient who was not administered the RNA vaccine, the PD-1 axis binding antagonist, and the chemotherapeutic treatment.

In some embodiments, the method described herein further comprises assessing overall survival (OS) of the human patient after treatment with the RNA vaccine, the PD-1 axis binding antagonist, and the chemotherapeutic treatment. OS is measured as the time from patient randomization to death from any cause. In some embodiments, administration of the RNA vaccine, the PD-1 axis binding antagonist, and the chemotherapeutic treatment results in an improvement in OS of the human patient as compared to OS of a corresponding human patient who was not administered the RNA vaccine, the PD-1 axis binding antagonist, and the chemotherapeutic treatment.

In some embodiments, the method described herein further comprises performing one or more clinical assessments of the human patient before, during and/or after treatment with the RNA vaccine, the PD-1 axis binding antagonist, and the chemotherapeutic treatment, wherein the one or more clinical assessments are selected from the group consisting of European Organisation for Research and Treatment of Cancer QLQ-C30 Questionnaire (EORTC QLQ C30), European Organisation for Research and Treatment of Cancer QLQ-PAN26 Questionnaire (EORTC QLQ PAN26), National Cancer Institute's Patient-Reported Outcomes Common Terminology Criteria for Adverse Events (PRO CTCAE), and European Organisation for Research and Treatment of Cancer Item Library 46 Questionnaire (EORTC IL46). In some embodiments, the clinical assessments are administered in the following order: EORTC QLQ-C30, EORTC QLQ-PAN26, PRO CTCAE, and EORTC IL46. The QLQ-C30 consists of 30 questions that assess five aspects of patient functioning (physical, emotional, role, cognitive, and social), three symptom scales (fatigue, nausea and vomiting, pain), global health and quality of life, and six single items (dyspnea, insomnia, appetite loss, constipation, diarrhea, and financial difficulties) with a recall period of the previous week. Scale scores can be obtained for the multi-item scales. The QLQ-PAN26 consists of 26 questions that assess nine pancreatic cancer-related and treatment-related symptoms (pain, eating-related items, cachexia, hepatic symptoms, side effects, altered bowel habits, ascites, indigestion, and flatulence) and five emotional domains specific to pancreatic cancer (body image, healthcare satisfaction, sexuality, fear of future health, an ability to plan for the future) (see, e.g., Mackay et al., HPB (Oxford); 24:443-451 (2022)). PRO CTCAE is used to characterize the presence, frequency of occurrence, severity, and/or degree of interference with daily function of 78 patient-reportable symptomatic treatment toxicities (see, e.g., Basch et al., J Natl Cancer Inst; 106: dju244 (2014); and Dueck et al., JAMA Oncol; 1:1051-1059 (2015)). The PRO-CTCAE contains 124 questions that are rated either dichotomously (for determination of presence vs. absence) or on a 5-point Likert scale (for determination of frequency of occurrence, severity, and interference with daily function). Treatment toxicities can occur with observable signs (e.g., vomiting) or non-observable symptoms (e.g., nausea). The standard PRO-CTCAE recall period is the previous 7 days. IL46 is a validated single-item question used to assess the overall impact of side effects and is used along with the PRO CTCAE to assess treatment tolerability. Symptomatic adverse events from the PRO-CTCAE item bank include, but may not be limited to, mouth/throat sores, nausea, vomiting, diarrhea, shortness of breath, cough, rash, hair loss, hand-foot syndrome, neuropathy, dizziness, headache, arthralgia, fatigue, bruising, chills, nosebleeds, injection-or IV-site pain. In some embodiments, administration of the RNA vaccine, the PD-1 axis binding antagonist, and the chemotherapeutic treatment results in an improvement in the one or more clinical assessments as compared to the one or more clinical assessments in the human patient prior to administration of the RNA vaccine, the PD-1 axis binding antagonist, and the chemotherapeutic treatment, and/or as compared to the one or more clinical assessments in a corresponding human patient who is not administered the RNA vaccine, the PD-1 axis binding antagonist, and the chemotherapeutic treatment.

In some embodiments, the method described herein further comprises assessing antigen-and/or tumor-specific T-cell responses in the human patient before, during and/or after treatment with the RNA vaccine, the PD-1 axis binding antagonist, and the chemotherapeutic treatment. In some embodiments, administration of the RNA vaccine, the PD-1 axis binding antagonist, and the chemotherapeutic treatment results in an improvement in antigen- and/or tumor-specific T-cell responses in the human patient as compared to prior to administration of the RNA vaccine, the PD-1 axis binding antagonist, and the chemotherapeutic treatment, and/or as compared to a corresponding human patient who was not administered the RNA vaccine, the PD-1 axis binding antagonist, and the chemotherapeutic treatment. In some embodiments, antigen- and/or tumor-specific T-cell responses in the human patient can be assessed by, for example, immune monitoring assays, including but not limited to: an IFN-γ release assay (e.g., ELISpot); tumor or immune biomarkers (e.g., PDL1, CD8) assays; whole exome sequencing, whole genome sequencing, or RNA sequencing and TCR sequencing to analyze mutational changes; monitor infiltration of immune cells and track antigen-specific T cells; analysis of circulating tumor DNA (ctDNA); analysis of cytokines, phenotype, and function of antigen-specific T cells, T-cell-receptor repertoire, immune cell subset number, proportion, functional status of (including T-cell subsets and myeloid-derived suppressor cells); titers of antibodies against reference antigens; single cell transcriptomic analysis; and detection of T cells specific to neoantigens encoded in the individualized cancer vaccine.

In some embodiments, the corresponding human patient is a human patient with a corresponding pancreatic cancer tumor. In some embodiments, the pancreatic cancer tumor is a PDAC tumor, and the corresponding human patient has a PDAC tumor. In some embodiments, the corresponding human patient was treated with a standard of care treatment for pancreatic cancer, PDAC, or resectable or resected PDAC. In some embodiments, the standard of care treatment comprises a gemcitabine combination therapy or an mFOLFIRINOX chemotherapy. mFOLFIRINOX has demonstrated a favorable benefit-risk profile, establishing it as current standard of care following resection of PDAC for fit patients (Conroy et al., N Engl J Med; 379:2395-2406 (2018); and Conroy et al., JAMA Oncol 2022;e223829 (2022), doi: 10.1001/jamaoncol.20223820 online ahead of print). mFOLFIRINOX therapy includes the following individual agents: leucovorin, 5 FU, irinotecan, and oxaliplatin. In some embodiments, the corresponding human patient was treated with a control treatment comprising an mFOLFIRINOX chemotherapy. In some embodiments, the mFOLFIRINOX chemotherapy comprises oxaliplatin at dose of about 85 mg/m², leucovorin at dose of about 400 mg/m², irinotecan at dose of about 150 mg/m², and 5-fluorouracil at dose of about 2400 mg/m², administered intravenously in 14-day cycles, on day 1 of each cycle for a total of up to 12 cycles. Compositions for Use in Treating Pancreatic Cancer

In one aspect, there is provided an individualized RNA vaccine for use in a method for treating a pancreatic cancer tumor in a human patient in need thereof, wherein the RNA vaccine is to be administered in combination with a PD-1 axis binding antagonist and a chemotherapeutic treatment according to the method described herein, wherein the RNA vaccine comprises one or more polynucleotides encoding one or more neoepitopes resulting from cancer-specific somatic mutations present in a pancreatic cancer tumor specimen obtained from the human patient. In another aspect, there is provided a use of an individualized RNA vaccine in the manufacture of a medicament for treating a pancreatic cancer tumor in a human patient in need thereof, wherein the RNA vaccine is to be administered in combination with a PD-1 axis binding antagonist and a chemotherapeutic treatment according to the method described herein, wherein the RNA vaccine comprises one or more polynucleotides encoding one or more neoepitopes resulting from cancer-specific somatic mutations present in a pancreatic cancer tumor specimen obtained from the human patient.

In one aspect, there is provided a PD-1 axis binding antagonist for use in a method for treating a pancreatic cancer tumor in a human patient in need thereof, wherein the PD-1 axis binding antagonist is to be administered in combination with an individualized RNA vaccine and a chemotherapeutic treatment according to the method described herein, wherein the RNA vaccine comprises one or more polynucleotides encoding one or more neoepitopes resulting from cancer-specific somatic mutations present in a pancreatic cancer tumor specimen obtained from the human patient. In another aspect, there is provided a use of a PD-1 axis binding antagonist in the manufacture of a medicament for treating a pancreatic cancer tumor in a human patient in need thereof, wherein the PD-1 axis binding antagonist is to be administered in combination with an individualized RNA vaccine and a chemotherapeutic treatment according to the method described herein, wherein the RNA vaccine comprises one or more polynucleotides encoding one or more neoepitopes resulting from cancer-specific somatic mutations present in a pancreatic cancer tumor specimen obtained from the human patient.

### Methods of Administration and Additional Therapies

The PD-1 axis binding antagonist, the RNA vaccine and the chemotherapeutic treatment may be administered by the same route of administration or by different routes of administration. In some embodiments, the PD-1 axis binding antagonist is administered intravenously, intramuscularly, subcutaneously, topically, orally, transdermally, intraperitoneally, intraorbitally, by implantation, by inhalation, intrathecally, intraventricularly, or intranasally. In some embodiments, the RNA vaccine is administered (e.g., in a lipoplex or lipid nanoparticle) intravenously, intramuscularly, subcutaneously, topically, orally, transdermally, intraperitoneally, intraorbitally, by implantation, by inhalation, intrathecally, intraventricularly, or intranasally. In some embodiments, the chemotherapeutic treatment is administered intravenously, intramuscularly, subcutaneously, topically, orally, transdermally, intraperitoneally, intraorbitally, by implantation, by inhalation, intrathecally, intraventricularly, or intranasally. In some embodiments, the RNA vaccine is administered (e.g., in a lipoplex) intravenously. some embodiments, the PD-1 axis binding antagonist, the RNA vaccine, and the chemotherapeutic treatment are administered intravenously. In some cases, the chemotherapeutic treatment may comprise a combination chemotherapy. In such cases, each individual agent within the combination may be administered by the same route of administration or by different routes of administration, e.g., as described above.

The PD-1 axis binding antagonist and the RNA vaccine may be administered in any order when administered on the same day, e.g., during the priming and/or boost phases of treatment. For example, the PD-1 axis binding antagonist and the RNA vaccine may be administered sequentially (at different times) or concurrently (at the same time). In some embodiments, the RNA vaccine is administered before the PD-1 axis binding antagonist. In some embodiments, the PD-1 axis binding antagonist and the RNA vaccine are in separate compositions. In some embodiments, the PD-1 axis binding antagonist and the RNA vaccine are in the same composition.

More than one RNA vaccine may be administered to a patient, e.g., the patient may be administered one RNA vaccine with a combination of neoepitopes and also administered a separate RNA vaccine with a different combination of neoepitopes. In some embodiments, a first RNA vaccine with, for example 5-20 or 10⁻²⁰ neoepitopes, is administered in combination with a second RNA vaccine with, for example 5-20 or 10⁻²⁰ different or alternative neoepitopes.

In some embodiments, the methods for treating pancreatic cancer provided herein may further comprise administering to the patient an additional therapy. The additional therapy may be radiation therapy, surgery (e.g., resection of a pancreatic cancer tumor), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy. In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery, e.g., resection of a pancreatic cancer tumor. In some embodiments, the additional therapy is a combination of radiation therapy and surgery, e.g., resection of a pancreatic cancer tumor. In some embodiments, the additional therapy is gamma irradiation.

### III. RNA Vaccines

Certain aspects of the present disclosure relate to individualized cancer vaccines (ICVs). In some embodiments, the individualized cancer vaccine is an RNA vaccine. Features of exemplary RNA vaccines are described infra. In some embodiments, the present disclosure provides an RNA polynucleotide comprising one or more of the features/sequences of the RNA vaccines described infra. In some embodiments, the RNA polynucleotide is a single-stranded mRNA polynucleotide. In other embodiments, the present disclosure provides a DNA polynucleotide encoding an RNA comprising one or more of the features/sequences of the RNA vaccines described infra.

Individualized cancer vaccines comprise individualized neoantigens (i.e., tumor-associated antigens (TAAs) that are specifically expressed in the patient's cancer) identified as having potential immunostimulatory activities. In the embodiments described herein, the individualized cancer vaccine is a nucleic acid, e.g., messenger RNA. Accordingly, without wishing to be bound by theory, it is believed that upon administration, the individualized cancer vaccine (e.g., an RNA vaccine of the disclosure) is taken up and translated by antigen presenting cells (APCs) and the expressed protein is presented via major histocompatibility complex (MHC) molecules on the surface of the APCs. This leads to an induction of both cytotoxic T-lymphocyte (CTL)- and memory T-cell-dependent immune responses against cancer cells expressing the TAA(s).

Individualized cancer vaccines (e.g., an RNA vaccine) typically include multiple neoantigen epitopes (“neoepitopes”), e.g., 2, 3, 4, 5, 6, 7, 8, 9, 10, 11, 12, 13, 14, 15, 16, 17, 18, 19, 20, 21, 22, 23, 24, 25, 26, 27, 28, 28, 29, or 30 neoepitopes or at least 2, 3, 4, 5, 6, 7, 8, 9, 10, 11, 12, 13, 14, 15, 16, 17, 18, 19, 20, 21, 22, 23, 24, 25, 26, 27, 28, 28, 29, or 30 neoepitopes, optionally with linker sequences between the individual neoepitopes. In some embodiments, a neoepitope as used herein refers to a novel epitope that is specific for a patient's cancer but not found in normal cells of the patient. In some embodiments, the neoepitope is presented to T cells when bound to MHC. In some embodiments, the individualized cancer vaccine also includes a 5′ mRNA cap analogue, a 5′ UTR, a signal sequence, a domain to facilitate antigen expression, a 3′ UTR, and/or a polyA tail. In some embodiments, the RNA vaccine comprises one or more polynucleotides encoding 5-20 or 10⁻²⁰ neoepitopes resulting from cancer-specific somatic mutations present in the tumor specimen. In some embodiments, the RNA vaccine comprises one or more polynucleotides encoding at least 5 neoepitopes resulting from cancer-specific somatic mutations present in the tumor specimen. In some embodiments, the RNA vaccine comprises one or more polynucleotides encoding 5-20 neoepitopes resulting from cancer-specific somatic mutations present in the tumor specimen. In some embodiments, the RNA vaccine comprises one or more polynucleotides encoding 5-10 neoepitopes resulting from cancer-specific somatic mutations present in the tumor specimen.

In some embodiments, the manufacture of an RNA vaccine of the present disclosure is a multi-step process, whereby somatic mutations in the patient's tumor are identified by next-generation sequencing (NGS) and immunogenic neoantigen epitopes (or “neoepitopes”) are predicted. The RNA cancer vaccine targeting the selected neoepitopes is manufactured on a per-patient basis. In some embodiments, the vaccine is an RNA-based cancer vaccine consisting of up to two messenger RNA molecules, each encoding up to 10 neoepitopes (for a total of up to 20 neoepitopes), which are specific to the patient's tumor.

In some embodiments, expressed non-synonymous mutations are identified by whole exome sequencing (WES) of tumor DNA and peripheral blood mononuclear cell (PBMC) DNA (as a source of healthy tissue from the patient) as well as tumor RNA sequencing (to assess expression). From the resulting list of mutant proteins, potential neoantigens are predicted using a bioinformatics workflow that ranks their likely immunogenicity on the basis of multiple factors, including the binding affinity of the predicted epitope to individual major histocompatibility complex (MHC) molecules, and the level of expression of the associated RNA. The mutation discovery, prioritization, and confirmation processes are complemented by a database that provides comprehensive information about expression levels of respective wild-type genes in healthy tissues. This information enables the development of a personalized risk mitigation strategy by removing target candidates with an unfavorable risk profile. Mutations occurring in proteins with a possible higher auto-immunity risk in critical organs are filtered out and not considered for vaccine production. In some embodiments, up to 20 MHCI and MHCII neoepitopes that are predicted to elicit CD8+ T-cell and/or CD4+ T-cell responses, respectively, for an individual patient are selected for inclusion into the vaccine. Vaccinating against multiple neoepitopes is expected to increase the breadth and magnitude of the overall immune response to individualized cancer vaccines and may help to mitigate the risk of immune escape, which can occur when tumors are exposed to the selective pressure of an effective immune response (Tran E, Robbins μF, Lu YC, et al. N Engl J Med 2016; 375:2255-62; Verdegaal EM, de Miranda NF, Visser M, et al. Nature 2016; 536:91-5).

In some embodiments, the RNA vaccine comprises one or more polynucleotide sequences encoding an amino acid linker. For example, amino acid linkers can be used between 2 tumor-specific neoepitope sequences, between a tumor-specific neoepitope sequence and a fusion protein tag (e.g., comprising sequence derived from an MHC complex polypeptide), or between a secretory signal peptide and a tumor-specific neoepitope sequence. In some embodiments, the RNA vaccine encodes multiple linkers. In some embodiments, the RNA vaccine comprises one or more polynucleotides encoding 5-20 neoepitopes resulting from cancer-specific somatic mutations present in the tumor specimen, and the polynucleotides encoding each epitope are separated by a polynucleotide encoding a linker sequence. In some embodiments, the RNA vaccine comprises one or more polynucleotides encoding 5-10 neoepitopes resulting from cancer-specific somatic mutations present in the tumor specimen, and the polynucleotides encoding each epitope are separated by a polynucleotide encoding a linker sequence. In some embodiments, polynucleotides encoding linker sequences are also present between the polynucleotides encoding an N-terminal fusion tag (e.g., a secretory signal peptide) and a polynucleotide encoding one of the neoepitopes and/or between a polynucleotide encoding one of the neoepitopes and the polynucleotides encoding a C-terminal fusion tag (e.g., comprising a portion of an MHC polypeptide). In some embodiments, two or more linkers encoded by the RNA vaccine comprise different sequences. In some embodiments, the RNA vaccine encodes multiple linkers, all of which share the same amino acid sequence.

In some embodiments, the RNA molecule further comprises a polynucleotide sequence encoding an amino acid linker; wherein the polynucleotide sequences encoding the amino acid linker and a first of the one or more neoepitopes form a first linker-neoepitope module; and wherein the polynucleotide sequences forming the first linker-neoepitope module are between the polynucleotide sequence encoding the secretory signal peptide and the polynucleotide sequence encoding the at least portion of the transmembrane and cytoplasmic domain of the MHC molecule in the 5′→3′ direction. In some embodiments, the RNA molecule further comprises, in the 5′→3′ direction: at least a second linker-neoepitope module, wherein the at least second linker-neoepitope module comprises a polynucleotide sequence encoding an amino acid linker and a polynucleotide sequence encoding a neoepitope; wherein the polynucleotide sequences forming the second linker-neoepitope module are between the polynucleotide sequence encoding the neoepitope of the first linker-neoepitope module and the polynucleotide sequence encoding the at least portion of the transmembrane and cytoplasmic domain of the MHC molecule in the 5′→3′ direction; and wherein the neoepitope of the first linker-neoepitope module is different from the neoepitope of the second linker-neoepitope module. In some embodiments, the RNA molecule comprises 5 linker-neoepitope modules, and wherein the 5 linker-neoepitope modules each encode a different neoepitope. In some embodiments, the RNA molecule comprises 10 linker-neoepitope modules, and wherein the 10 linker-neoepitope modules each encode a different neoepitope. In some embodiments, the RNA molecule comprises 20 linker-neoepitope modules, and wherein the 20 linker-neoepitope modules each encode a different neoepitope. In some embodiments, the RNA molecule further comprises a second polynucleotide sequence encoding an amino acid linker, wherein the second polynucleotide sequence encoding the amino acid linker is between the polynucleotide sequence encoding the neoepitope that is most distal in the 3′ direction and the polynucleotide sequence encoding the at least portion of the transmembrane and cytoplasmic domain of the MHC molecule.

A variety of linker sequences are known in the art. In some embodiments, the linker is a flexible linker. In some embodiments, the linker comprises G, S, A, and/or T residues. In some embodiments, the linker consists of glycine and serine residues. In some embodiments, the linker is between about 5 and about 20 amino acids or between about 5 and about 12 amino acids in length, e.g., about 5, about 6, about 7, about 8, about 9, about 10, about 11, about 12, about 13, about 14, about 15, about 16, about 17, about 18, about 19, or about 20 amino acids in length. In some embodiments, the linker comprises the sequence GGSGGGGSGG (SEQ ID NO:39). In some embodiments, the linker of the RNA vaccine comprises the sequence GGCGGCUCUGGAGGAGGCGGCUCCGGAGGC (SEQ ID NO:37). In some embodiments, the linker of the RNA vaccine is encoded by DNA comprising the sequence

| (SEQ ID NO: 38) | |
|---|---|
| | GGCGGCTCTGGAGGAGGCGGCTCCGGAGGC. |

In some embodiments, the RNA vaccine comprises a 5′ cap. The basic mRNA cap structure is known to contain a 5′-5′ triphosphate linkage between 2 nucleosides (e.g., two guanines) and a 7-methyl group on the distal guanine, i.e., m⁷GpppG. Exemplary cap structures can be found, e.g., in U.S. Pat. Nos. 8,153,773 and 9,295,717 and Kuhn, A. N. et al. (2010) Gene Ther. 17:961-971. In some embodiments, the 5′ cap has the structure m^{27,2}′-OGppspG. In some embodiments, the 5′ cap is a beta-S-ARCA cap. The S-ARCA cap structure includes a 2′-O methyl substitution (e.g., at the C2′ position of the m⁷G) and an S-substitution at one or more of the phosphate groups. In some embodiments, the 5′ cap comprises the structure:

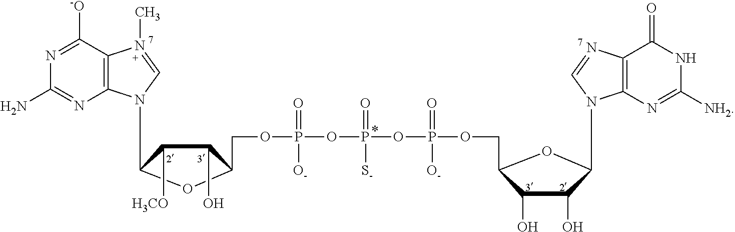

In some embodiments, the 5′ cap is the DI diastereoisomer of beta-S-ARCA (see, e.g., U.S. Pat. No. 9,295,717). The * in the above structure indicates a stereogenic P center, which can exist in two diastereoisomers (designated D1 and D2). The D1 diastereomer of beta-S-ARCA or beta-S-ARCA (DI) is the diastereomer of beta-S-ARCA which elutes first on an HPLC column compared to the D2 diastereomer of beta-S-ARCA (beta-S-ARCA (D2)) and thus exhibits a shorter retention time. The HPLC preferably is an analytical HPLC. In one embodiment, a Supelcosil LC-18-T RP column, preferably of the format: 5 μm, 4.6×250 mm is used for separation, whereby a flow rate of 1.3 ml/min can be applied. In one embodiment, a gradient of methanol in ammonium acetate, for example, a 0-25% linear gradient of methanol in 0.05 M ammonium acetate, pH=5.9, within 15 min is used. UV-detection (VWD) can be performed at 260 nm and fluorescence detection (FLD) can be performed with excitation at 280 nm and detection at 337 nm.

In some embodiments, the RNA vaccine comprises a 5′ UTR. Certain untranslated sequences found 5′ to protein-coding sequences in mRNAs have been shown to increase translational efficiency. See, e.g., Kozak, M. (1987) J. Mol. Biol. 196:947-950. In some embodiments, the 5′ UTR comprises sequence from the human alpha globin mRNA. In some embodiments, the RNA vaccine comprises a 5′ UTR sequence of UUCUUCUGGUCCCCACAGACUCAGAGAGAACCCGCCACC (SEQ ID NO:23). In some embodiments, the 5′ UTR sequence of the RNA vaccine is encoded by DNA comprising the sequence TTCTTCTGGTCCCCACAGACTCAGAGAGAACCCGCCACC (SEQ ID NO:24). In some embodiments, the 5′ UTR sequence of RNA vaccine comprises the sequence GGCGAACUAGUAUUCUUCUGGUCCCCACAGACUCAGAGAGAACCCGCCACC (SEQ ID NO:21). In some embodiments, the 5′ UTR sequence of RNA vaccine is encoded by DNA comprising the sequence

(SEQ ID NO: 22)
GGCGAACTAGTATTCTTCTGGTCCCCACAGACTCAGAGAGAACCCGCCAC
C.

In some embodiments of the methods provided herein, the constant region of an exemplary RNA vaccine comprises the ribonucleotide sequence (5′→3′) of SEQ ID NO: 42. The linkage between the first two G residues is the unusual bond (5′→5′)-ppsp-, e.g., as shown in Table 4. “N” refers to the position of polynucleotide sequence(s) encoding one or more (e.g., 1-20) neoepitopes (separated by optional linkers). The insertion site for tumor-specific sequences (C131-A132; marked in bold text) is depicted in bold text. See Table 4 for the modified bases and uncommon links in the exemplary RNA sequence.

TABLE 4

| Type | Location | Description |
|---|---|---|
| Modified Base | G1 | m₂^{7·2′·O} G |
| Uncommon Link | G1-G2 | (5′→5′)-ppₛp- |
| Uncommon Link | C131-A132 | Insertion site for tumor-specific |
| | | sequences |

In some embodiments, the RNA vaccine comprises a polynucleotide sequence encoding a secretory signal peptide. As is known in the art, a secretory signal peptide is an amino acid sequence that directs a polypeptide to be trafficked from the endoplasmic reticulum and into the secretory pathway upon translation. In some embodiments, the signal peptide is derived from a human polypeptide, such as an MHC polypeptide. See, e.g., Kreiter, S. et al. (2008) J. Immunol. 180:309-318, which describes an exemplary secretory signal peptide that improves processing and presentation of MHC Class I and II epitopes in human dendritic cells. In some embodiments, upon translation, the signal peptide is N-terminal to one or more neoepitope sequence(s) encoded by the RNA vaccine. In some embodiments, the secretory signal peptide comprises the sequence MRVMAPRTLILLLSGALALTETWAGS (SEQ ID NO:27). In some embodiments, the secretory signal peptide of the RNA vaccine comprises the sequence AUGAGAGUGAUGGCCCCCAGAACCCUGAUCCUGCUGCUGUCUGGCGCCCUGGCCCUGA CAGAGACAUGGGCCGGAAGC (SEQ ID NO:25). In some embodiments, the secretory signal peptide of the RNA vaccine is encoded by DNA comprising the sequence

(SEQ ID NO: 26)
ATGAGAGTGATGGCCCCCAGAACCCTGATCCTGCTGCTGTCTGGCGCCCT
GGCCCTGACAGAGACATGGGCCGGAAGC.

In some embodiments, the RNA vaccine comprises a polynucleotide sequence encoding at least a portion of a transmembrane and/or cytoplasmic domain. In some embodiments, the transmembrane and/or cytoplasmic domains are from the transmembrane/cytoplasmic domains of an MHC molecule. The term “major histocompatibility complex” and the abbreviation “MHC” relate to a complex of genes which occurs in all vertebrates. The function of MHC proteins or molecules in signaling between lymphocytes and antigen-presenting cells in normal immune responses involves them binding peptides and presenting them for possible recognition by T-cell receptors (TCR). MHC molecules bind peptides in an intracellular processing compartment and present these peptides on the surface of antigen-presenting cells to T cells. The human MHC region, also referred to as HLA, is located on chromosome 6 and comprises the class I region and the class II region. The class I alpha chains are glycoproteins having a molecular weight of about 44 kDa. The polypeptide chain has a length of somewhat more than 350 amino acid residues. It can be divided into three functional regions: an external, a transmembrane and a cytoplasmic region. The external region has a length of 283 amino acid residues and is divided into three domains, alphal, alpha2 and alpha3. The domains and regions are usually encoded by separate exons of the class I gene. The transmembrane region spans the lipid bilayer of the plasma membrane. It consists of 23 usually hydrophobic amino acid residues which are arranged in an alpha helix. The cytoplasmic region, i.e., the part which faces the cytoplasm and which is connected to the transmembrane region, typically has a length of 32 amino acid residues and is able to interact with the elements of the cytoskeleton. The alpha chain interacts with beta2-microglobulin and thus forms alpha-beta2 dimers on the cell surface. The term “MHC class II” or “class II” relates to the major histocompatibility complex class II proteins or genes. Within the human MHC class II region there are the DP, DQ, and DR subregions for class II alpha chain genes and beta chain genes (i.e., DPalpha, DPbeta, DQalpha, DQbeta, DRalpha and DRbeta). Class II molecules are heterodimers each consisting of an alpha chain and a beta chain. Both chains are glycoproteins having a molecular weight of 31-34 kDa (a) or 26-29 kDA (beta). The total length of the alpha chains varies from 229 to 233 amino acid residues, and that of the beta chains from 225 to 238 residues. Both alpha and beta chains consist of an external region, a connecting peptide, a transmembrane region, and a cytoplasmic tail. The external region consists of two domains, alpha1 and alpha2 or beta1 and beta2. The connecting peptide is respectively beta and 9 residues long in alpha and beta chains. It connects the two domains to the transmembrane region which consists of 23 amino acid residues both in alpha chains and in beta chains. The length of the cytoplasmic region, i.e., the part which faces the cytoplasm and which is connected to the transmembrane region, varies from 3 to 16 residues in alpha chains and from 8 to 20 residues in beta chains. Exemplary transmembrane/cytoplasmic domain sequences are described in U.S. Pat. Nos. 8,178,653 and 8,637,006. In some embodiments, upon translation, the transmembrane and/or cytoplasmic domain is C-terminal to one or more neoepitope sequence(s) encoded by the RNA vaccine. In some embodiments, the transmembrane and/or cytoplasmic domain of the MHC molecule encoded by the RNA vaccine comprises the sequence IVGIVAGLAVLAVVVIGAVVATVMCRRKSSGGKGGSYSQAASSDSAQGSDVSLTA (SEQ ID NO: 30). In some embodiments, the transmembrane and/or cytoplasmic domain of the MHC molecule comprises the sequence AUCGUGGGAAUUGUGGCAGGACUGGCAGUGCUGGCCGUGGUGGUGAUCGGAGCCGUG GUGGCUACCGUGAUGUGCAGACGGAAGUCCAGCGGAGGCAAGGGCGGCAGCUACAGC CAGGCCGCCAGCUCUGAUAGCGCCCAGGGCAGCGACGUGUCACUGACAGCC (SEQ ID NO: 28). In some embodiments, the transmembrane and/or cytoplasmic domain of the MHC molecule is encoded by DNA comprising the sequence

(SEQ ID NO: 29)
ATCGTGGGAATTGTGGCAGGACTGGCAGTGCTGGCCGTGGTGGTGATCGG
AGCCGTGGTGGCTACCGTGATGTGCAGACGGAAGTCCAGCGGAGGCAAGG
GCGGCAGCTACAGCCAGGCCGCCAGCTCTGATAGCGCCCAGGGCAGCGAC
GTGTCACTGACAGCC.

In some embodiments, the RNA vaccine comprises both a polynucleotide sequence encoding a secretory signal peptide that is N-terminal to the one or more neoepitope sequence(s) and a polynucleotide sequence encoding a transmembrane and/or cytoplasmic domain that is C-terminal to the one or more neoepitope sequence(s). Combining such sequences has been shown to improve processing and presentation of MHC Class I and II epitopes in human dendritic cells. See, e.g., Kreiter, S. et al. (2008) J. Immunol. 180:309-318.

In myeloid DCs, the RNA is released into the cytosol and translated into a poly-neoepitopic peptide. The polypeptide contains additional sequences to enhance antigen presentation. In some embodiments, a signal sequence (sec) from the MHCI heavy chain at the N-terminal of the polypeptide is used to target the nascent molecule to the endoplasmic reticulum, which has been shown to enhance MHCI presentation efficiency. Without wishing to be bound by theory, it is believed that the transmembrane and cytoplasmic domains of MHCI heavy chain guide the polypeptide to the endosomal/lysosomal compartments that were shown to improve MHCII presentation.

In some embodiments, the RNA vaccine comprises a 3′UTR. Certain untranslated sequences found 3′ to protein-coding sequences in mRNAs have been shown to improve RNA stability, translation, and protein expression. Polynucleotide sequences suitable for use as 3′ UTRs are described, for example, in PG Pub. No. US20190071682. In some embodiments, the 3′ UTR comprises the 3′ untranslated region of AES or a fragment thereof and/or the non-coding RNA of the mitochondrially encoded 12S RNA. The term “AES” relates to Amino-Terminal Enhancer Of Split and includes the AES gene (see, e.g., NCBI Gene ID: 166). The protein encoded by this gene belongs to the groucho/TLE family of proteins, can function as a homooligomer or as a heteroologimer with other family members to dominantly repress the expression of other family member genes. An exemplary AES mRNA sequence is provided in NCBI Ref. Seq. Accession NO. NM_198969. The term “MT_RNR1” relates to Mitochondrially Encoded 12S RNA and includes the MT_RNRI gene (see, e.g., NCBI Gene ID: 4549). This RNA gene belongs to the Mt_rRNA class. Diseases associated with MT-RNR1 include restrictive cardiomyopathy and auditory neuropathy. Among its related pathways are Ribosome biogenesis in eukaryotes and CFTR translational fidelity (class I mutations). An exemplary MT_RNR1 RNA sequence is present within the sequence of NCBI Ref. Seq. Accession NO. NC_012920. In some embodiments, the 3′ UTR of the RNA vaccine comprises the sequence CUGGUACUGCAUGCACGCAAUGCUAGCUGCCCCUUUCCCGUCCUGGGUACCCCGAGUC UCCCCCGACCUCGGGUCCCAGGUAUGCUCCCACCUCCACCUGCCCCACUCACCACCUCU GCUAGUUCCAGACACCUCC (SEQ ID NO:33). In some embodiments, the 3′ UTR of the RNA vaccine comprises the sequence CAAGCACGCAGCAAUGCAGCUCAAAACGCUUAGCCUAGCCACACCCCCACGGGAAACA GCAGUGAUUAACCUUUAGCAAUAAACGAAAGUUUAACUAAGCUAUACUAACCCCAGG GUUGGUCAAUUUCGUGCCAGCCACACCG (SEQ ID NO:35). In some embodiments, the 3′ UTR of the RNA vaccine comprises the sequence CUGGUACUGCAUGCACGCAAUGCUAGCUGCCCCUUUCCCGUCCUGGGUACCCCGAGUC UCCCCCGACCUCGGGUCCCAGGUAUGCUCCCACCUCCACCUGCCCCACUCACCACCUCU GCUAGUUCCAGACACCUCC (SEQ ID NO:33) and the sequence CAAGCACGCAGCAAUGCAGCUCAAAACGCUUAGCCUAGCCACACCCCCACGGGAAACA GCAGUGAUUAACCUUUAGCAAUAAACGAAAGUUUAACUAAGCUAUACUAACCCCAGG GUUGGUCAAUUUCGUGCCAGCCACACCG (SEQ ID NO:35). In some embodiments, the 3′ UTR of the RNA vaccine comprises the sequence CUCGAGCUGGUACUGCAUGCACGCAAUGCUAGCUGCCCCUUUCCCGUCCUGGGUACCC CGAGUCUCCCCCGACCUCGGGUCCCAGGUAUGCUCCCACCUCCACCUGCCCCACUCACC ACCUCUGCUAGUUCCAGACACCUCCCAAGCACGCAGCAAUGCAGCUCAAAACGCUUAG CCUAGCCACACCCCCACGGGAAACAGCAGUGAUUAACCUUUAGCAAUAAACGAAAGUU UAACUAAGCUAUACUAACCCCAGGGUUGGUCAAUUUCGUGCCAGCCACACCGAGACCU GGUCCAGAGUCGCUAGCCGCGUCGCU (SEQ ID NO:31). In some embodiments, the 3′ UTR of the RNA vaccine is encoded by DNA comprising the sequence CTGGTACTGCATGCACGCAATGCTAGCTGCCCCTTTCCCGTCCTGGGTACCCCGAGTCTC CCCCGACCTCGGGTCCCAGGTATGCTCCCACCTCCACCTGCCCCACTCACCACCTCTGCT AGTTCCAGACACCTCC (SEQ ID NO:34). In some embodiments, the 3′ UTR of the RNA vaccine is encoded by DNA comprising the sequence CAAGCACGCAGCAATGCAGCTCAAAACGCTTAGCCTAGCCACACCCCCACGGGAAACAG CAGTGATTAACCTTTAGCAATAAACGAAAGTTTAACTAAGCTATACTAACCCCAGGGTTG GTCAATTTCGTGCCAGCCACACCG (SEQ ID NO:36). In some embodiments, the 3′ UTR of the RNA vaccine is encoded by DNA comprising the sequence CTGGTACTGCATGCACGCAATGCTAGCTGCCCCTTTCCCGTCCTGGGTACCCCGAGTCTC CCCCGACCTCGGGTCCCAGGTATGCTCCCACCTCCACCTGCCCCACTCACCACCTCTGCT AGTTCCAGACACCTCC (SEQ ID NO:34) and the sequence CAAGCACGCAGCAATGCAGCTCAAAACGCTTAGCCTAGCCACACCCCCACGGGAAACAG CAGTGATTAACCTTTAGCAATAAACGAAAGTTTAACTAAGCTATACTAACCCCAGGGTTG GTCAATTTCGTGCCAGCCACACCG (SEQ ID NO:36). In some embodiments, the 3′ UTR of the RNA vaccine is encoded by DNA comprising the sequence

(SEQ ID NO: 32)
CTGGTACTGCATGCACGCAATGCTAGCTGCCCCTTTCCCGTCCTGGGTAC
CCCGAGTCTCCCCCGACCTCGGGTCCCAGGTATGCTCCCACCTCCACCTG
CCCCACTCACCACCTCTGCTAGTTCCAGACACCTCCCAAGCACGCAGCAA
TGCAGCTCAAAACGCTTAGCCTAGCCACACCCCCACGGGAAACAGCAGTG
ATTAACCTTTAGCAATAAACGAAAGTTTAACTAAGCTATACTAACCCCAG
GGTTGGTCAATTTCGTGCCAGCCACACCGAGACCTGGTCCAGAGTCGCTA
GCCGCGTCGCT.

In some embodiments, the RNA vaccine comprises a poly(A) tail at its 3′end. In some embodiments, the poly(A) tail comprises more than 50 or more than 100 adenine nucleotides. For example, in some embodiments, the poly(A) tail comprises 120 adenine nucleotides. This poly(A) tail has been demonstrated to enhance RNA stability and translation efficiency (Holtkamp, S. et al. (2006) Blood 108:4009-4017). In some embodiments, the RNA comprising a poly(A) tail is generated by transcribing a DNA molecule comprising in the 5′→3′ direction of transcription, a polynucleotide sequence that encodes at least 50, 100, or 120 adenine consecutive nucleotides and a recognition sequence for a type IIS restriction endonuclease. Exemplary poly(A) tail and 3′ UTR sequences that improve translation are found, e.g., in U.S. Pat. No. 9,476,055.

In some embodiments, an RNA vaccine or molecule of the present disclosure comprises the general structure (in the 5′→3′ direction): (1) a 5′ cap; (2) a 5′ untranslated region (UTR); (3) a polynucleotide sequence encoding a secretory signal peptide; (4) a polynucleotide sequence encoding at least a portion of a transmembrane and cytoplasmic domain of a major histocompatibility complex (MHC) molecule; (5) a 3′ UTR comprising: (a) a 3′ untranslated region of an Amino-Terminal Enhancer of Split (AES) mRNA or a fragment thereof; and (b) non-coding RNA of a mitochondrially encoded 12S RNA or a fragment thereof; and (6) a poly(A) sequence. In some embodiments, an RNA vaccine or molecule of the present disclosure comprises, in the 5′→3′ direction: the polynucleotide sequence GGCGAACUAGUAUUCUUCUGGUCCCCACAGACUCAGAGAGAACCCGCCACCAUGAGAG UGAUGGCCCCCAGAACCCUGAUCCUGCUGCUGUCUGGCGCCCUGGCCCUGACAGAGAC AUGGGCCGGAAGC (SEQ ID NO:19); and the polynucleotide sequence AUCGUGGGAAUUGUGGCAGGACUGGCAGUGCUGGCCGUGGUGGUGAUCGGAGCCGUG GUGGCUACCGUGAUGUGCAGACGGAAGUCCAGCGGAGGCAAGGGCGGCAGCUACAGC CAGGCCGCCAGCUCUGAUAGCGCCCAGGGCAGCGACGUGUCACUGACAGCCUAGUAAC UCGAGCUGGUACUGCAUGCACGCAAUGCUAGCUGCCCCUUUCCCGUCCUGGGUACCCC GAGUCUCCCCCGACCUCGGGUCCCAGGUAUGCUCCCACCUCCACCUGCCCCACUCACCA CCUCUGCUAGUUCCAGACACCUCCCAAGCACGCAGCAAUGCAGCUCAAAACGCUUAGC CUAGCCACACCCCCACGGGAAACAGCAGUGAUUAACCUUUAGCAAUAAACGAAAGUUU AACUAAGCUAUACUAACCCCAGGGUUGGUCAAUUUCGUGCCAGCCACACCGAGACCUG GUCCAGAGUCGCUAGCCGCGUCGCU (SEQ ID NO:20). Advantageously, RNA vaccines comprising this combination and orientation of structures or sequences are characterized by one or more of: improved RNA stability, enhanced translational efficiency, improved antigen presentation and/or processing (e.g., by DCs), and increased protein expression.

In some embodiments, an RNA vaccine or molecule of the present disclosure comprises the sequence (in the 5′→3′ direction) of SEQ ID NO:42. In some embodiments, N refers to a polynucleotide sequence encoding at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 different neoepitopes. In some embodiments, N refers to a polynucleotide sequence encoding one or more linker-epitope modules (e.g., at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 different linker-epitope modules). In some embodiments, N refers to a polynucleotide sequence encoding one or more linker-epitope modules (e.g., at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 different linker-epitope modules) and an additional amino acid linker at the 3′ end.

In some embodiments, the RNA vaccine or molecule further comprises a polynucleotide sequence encoding at least one neoepitope; wherein the polynucleotide sequence encoding the at least one neoepitope is between the polynucleotide sequence encoding the secretory signal peptide and the polynucleotide sequence encoding the at least portion of the transmembrane and cytoplasmic domain of the MHC molecule in the 5′→3′ direction. In some embodiments, the RNA molecule comprises a polynucleotide sequence encoding at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or 20 different neoepitopes.

In some embodiments, the RNA vaccine or molecule further comprises, in the 5′→3′ direction: a polynucleotide sequence encoding an amino acid linker; and a polynucleotide sequence encoding a neoepitope. In some embodiments, the polynucleotide sequences encoding the amino acid linker and the neoepitope form a linker-neoepitope module (e.g., a continuous sequence in the 5′→3′ direction in the same open-reading frame). In some embodiments, the polynucleotide sequences forming the linker-neoepitope module are between the polynucleotide sequence encoding the secretory signal peptide and the polynucleotide sequence encoding the at least portion of the transmembrane and cytoplasmic domain of the MHC molecule, or between the sequences of SEQ ID NO: 19 and SEQ ID NO:20, in the 5′→3′ direction. In some embodiments, the RNA vaccine or molecule comprises 2, 3, 4, 5, 6, 7, 8, 9, 10, 11, 12, 13, 14, 15, 16, 17, 18, 19, 20, 21, 22, 23, 24, 25, 26, 27, 28, 28, 29, or 30 linker-epitope modules. In some embodiments, each of the linker-epitope modules encodes a different neoepitope. In some embodiments, the RNA vaccine or molecule comprises 2, 3, 4, 5, 6, 7, 8, 9, 10, 11, 12, 13, 14, 15, 16, 17, 18, 19, or 20 linker-epitope modules, and the RNA vaccine or molecule comprises polynucleotides encoding at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or 20 different neoepitopes. In some embodiments, the RNA vaccine or molecule comprises 5, 10, or 20 linker-epitope modules. In some embodiments, each of the linker-epitope modules encodes a different neoepitope. In some embodiments, the linker-epitope modules form a continuous sequence in the 5′→3′ direction in the same open-reading frame. In some embodiments, the polynucleotide sequence encoding the linker of the first linker-epitope module is 3′ of the polynucleotide sequence encoding the secretory signal peptide. In some embodiments, the polynucleotide sequence encoding the neoepitope of the last linker-epitope module is 5′ of the polynucleotide sequence encoding the at least portion of the transmembrane and cytoplasmic domain of the MHC molecule.

In some embodiments, the RNA vaccine is at least 800 nucleotides, at least 1000 nucleotides, or at least 1200 nucleotides in length. In some embodiments, the RNA vaccine is less than 2000 nucleotides in length. In some embodiments, the RNA vaccine is at least 800 nucleotides but less than 2000 nucleotides in length, at least 1000 nucleotides but less than 2000 nucleotides in length, at least 1200 nucleotides but less than 2000 nucleotides in length, at least 1400 nucleotides but less than 2000 nucleotides in length, at least 800 nucleotides but less than 1400 nucleotides in length, or at least 800 nucleotides but less than 2000 nucleotides in length. For example, the constant regions of an RNA vaccine comprising the elements described above are approximately 800 nucleotides in length. In some embodiments, an RNA vaccine comprising 5 tumor-specific neoepitopes (e.g., each encoding 27 amino acids) is greater than 1300 nucleotides in length. In some embodiments, an RNA vaccine comprising 10 tumor-specific neoepitopes (e.g., each encoding 27 amino acids) is greater than 1800 nucleotides in length.

In some embodiments, the one or more polynucleotides of the RNA vaccine are formulated with one or more lipids. In some embodiments, the RNA vaccine is formulated as a lipid nanoparticle, wherein the one or more polynucleotides of the RNA vaccine and one or more lipids form the lipid nanoparticle. In some embodiments, the RNA vaccine is formulated as a lipoplex, wherein the one or more polynucleotides of the RNA vaccine and one or more lipids form the lipoplex. In some embodiments, a lipoplex formulation for the RNA (RNA-Lipoplex) is used to enable IV delivery of an RNA vaccine of the present disclosure. In some embodiments, a lipoplex formulation for the RNA cancer vaccine comprising the synthetic cationic lipid

(R)-N,N,N-trimethyl-2,3-dioleyloxy-1-propanaminium chloride (DOTMA) and the phospholipid 1,2-dioleoyl-sn-glycero-3-phosphoethanolamine (DOPE) is used, e.g., to enable IV delivery. The DOTMA/DOPE liposomal component has been optimized for IV delivery and targeting of antigen-presenting cells in the spleen and other lymphoid organs.

In some embodiments, the lipid nanoparticles or the lipoplexes comprise at least one cationic lipid. The cationic lipid can be monocationic or polycationic. Any cationic amphiphilic molecule, e.g., a molecule which comprises at least one hydrophilic and lipophilic moiety is a cationic lipid within the meaning of the present invention. In one embodiment, the positive charges are contributed by the at least one cationic lipid and the negative charges are contributed by the RNA. In one embodiment, the lipid nanoparticle or lipoplex comprise at least one helper lipid. The helper lipid may be a neutral or an anionic lipid. The helper lipid may be a natural lipid, such as a phospholipid or an analogue of a natural lipid, or a fully synthetic lipid, or lipid-like molecule, with no similarities with natural lipids. In one embodiment, the cationic lipid and/or the helper lipid is a bilayer forming lipid.

In one embodiment, the at least one cationic lipid comprises 1,2-di-O-octadecenyl-3-trimethylammonium propane (DOTMA) or analogs or derivatives thereof and/or 1,2-dioleoyl-3-trimethylammonium-propane (DOTAP) or analogs or derivatives thereof.

In one embodiment, the at least one helper lipid comprises 1,2-di-(9Z-octadecenoyl)-sn-glycero-3-phosphoethanolamine (DOPE) or analogs or derivatives thereof, cholesterol (Chol) or analogs or derivatives thereof and/or 1,2-dioleoyl-sn-glycero-3-phosphocholine (DOPC) or analogs or derivatives thereof.

In one embodiment, the molar ratio of the at least one cationic lipid to the at least one helper lipid is from 10:0 to 3:7, preferably 9:1 to 3:7, 4:1 to 1:2, 4:1 to 2:3, 7:3 to 1:1, or 2:1 to 1:1, preferably about 1:1. In one embodiment, in this ratio, the molar amount of the cationic lipid results from the molar amount of the cationic lipid multiplied by the number of positive charges in the cationic lipid.

In one embodiment, the lipid is comprised in a vesicle encapsulating said RNA. The vesicle may be a multilamellar vesicle, an unilamellar vesicle, or a mixture thereof. The vesicle may be a lipoplex or lipid nanoparticle.

RNA vaccine formulations with one or more lipids described herein can be formed by adjusting a positive to negative charge, depending on the (+/−) charge ratio of a cationic lipid to RNA and mixing the RNA and the cationic lipid. The +/−charge ratio of the cationic lipid to the RNA in the lipid nanoparticles or the lipoplexes described herein can be calculated by the following equation. (+/−charge ratio)=[(cationic lipid amount (mol))*(the total number of positive charges in the cationic lipid)]: [(RNA amount (mol))*(the total number of negative charges in RNA)]. The RNA amount and the cationic lipid amount can be easily determined by one skilled in the art in view of a loading amount upon preparation of the nanoparticles or lipoplexes. For further descriptions of exemplary nanoparticles and lipoplexes, see, e.g., PG Pub. No. US20150086612.

In one embodiment, the overall charge ratio of positive charges to negative charges in the lipid nanoparticle (e.g., at physiological pH) is between 1.4:1 and 1:8, preferably between 1.2:1 and 1:4, e.g. between 1:1 and 1:3 such as between 1:1.2 and 1:2, 1:1.2 and 1:1.8, 1:1.3 and 1:1.7, in particular between 1:1.4 and 1:1.6, such as about 1:1.5. In some embodiments, at physiological pH the overall charge ratio of positive charges to negative charges of the lipid nanoparticles is between 1:1.2 (0.83) and 1:2 (0.5). In some embodiments, at physiological pH the overall charge ratio of positive charges to negative charges of the lipid nanoparticle is between 1.6:2 (0.8) and 1:2 (0.5) or between 1.6:2 (0.8) and 1.1:2 (0.55). In some embodiments, at physiological pH the overall charge ratio of positive charges to negative charges of the lipid nanoparticle is 1.3:2 (0.65). In some embodiments, at physiological pH the overall charge ratio of positive charges to negative charges of the lipid nanoparticle is not lower than 1.0:2.0. In some embodiments, at physiological pH the overall charge ratio of positive charges to negative charges of the lipid nanoparticle is not higher than 1.9:2.0. In some embodiments, at physiological pH the overall charge ratio of positive charges to negative charges of the lipid nanoparticle is not lower than 1.0:2.0 and not higher than 1.9:2.0.

In another embodiment, the overall charge ratio of positive charges to negative charges in the lipoplex (e.g., at physiological pH) is between 1.4:1 and 1:8, preferably between 1.2:1 and 1:4, e.g., between 1:1 and 1:3 such as between 1:1.2 and 1:2, 1:1.2 and 1:1.8, 1:1.3 and 1:1.7, in particular between 1:1.4 and 1:1.6, such as about 1:1.5. In some embodiments, at physiological pH the overall charge ratio of positive charges to negative charges of the lipoplex is between 1:1.2 (0.83) and 1:2 (0.5). In some embodiments, at physiological pH the overall charge ratio of positive charges to negative charges of the lipoplex is between 1.6:2 (0.8) and 1:2 (0.5) or between 1.6:2 (0.8) and 1.1:2 (0.55). In some embodiments, at physiological pH the overall charge ratio of positive charges to negative charges of the lipoplex is 1.3:2 (0.65). In some embodiments, at physiological pH the overall charge ratio of positive charges to negative charges of the lipoplex is not lower than 1.0:2.0. In some embodiments, at physiological pH the overall charge ratio of positive charges to negative charges of the lipoplex is not higher than 1.9:2.0. In some embodiments, at physiological pH the overall charge ratio of positive charges to negative charges of the lipoplex is not lower than 1.0:2.0 and not higher than 1.9:2.0.

In one embodiment, the lipoplexes or lipid nanoparticles comprise DOTMA and DOPE in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2. In one embodiment, the lipoplexes or lipid nanoparticles comprise DOTMA and Cholesterol in a molar ratio of 10:0 to 1:9, preferably 8:2 to 3:7, and more preferably of 7:3 to 5:5, wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2. In one embodiment, the lipoplexes or lipid nanoparticles comprise DOTAP and DOPE in a molar ratio of 10:0 to 1:9, preferably 8:2 to 3:7, and more preferably of 7:3 to 5:5, wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2. In one embodiment, the lipoplexes or lipid nanoparticles comprise DOTMA and DOPE in a molar ratio of 2:1 to 1:2, preferably 2:1 to 1:1, wherein the charge ratio of positive charges in DOTMA to negative charges in the RNA is 1.4:1 or less. In one embodiment, the lipoplexes or lipid nanoparticles comprise DOTMA and cholesterol in a molar ratio of 2:1 to 1:2, preferably 2:1 to 1:1, wherein the charge ratio of positive charges in DOTMA to negative charges in the RNA is 1.4:1 or less. In one embodiment, the lipoplexes or lipid nanoparticles comprise DOTAP and DOPE in a molar ratio of 2:1 to 1:2, preferably 2:1 to 1:1, wherein the charge ratio of positive charges in DOTAP to negative charges in the RNA is 1.4:1 or less.

In one embodiment, the zeta potential of the lipoplexes or lipid nanoparticles is −5 or less, −10 or less, −15 or less, −20 or less or −25 or less. In various embodiments, the zeta potential of the lipoplexes or lipid nanoparticles is −35 or higher, −30 or higher or −25 or higher. In one embodiment, the lipoplexes or lipid nanoparticles have a zeta potential from 0 mV to −50 mV, preferably 0 mV to −40 mV or −10 mV to −30 mV.

In some embodiments, the polydispersity index of the lipoplexes or lipid nanoparticles is 0.5 or less, 0.4 or less, or 0.3 or less, as measured by dynamic light scattering.

In some embodiments, the lipoplexes or lipid nanoparticles have an average diameter in the range of about 50 nm to about 1000 nm, from about 100 nm to about 800 nm, from about 200 nm to about 600 nm, from about 250 nm to about 700 nm, or from about 250 nm to about 550 nm, as measured by dynamic light scattering.

In some embodiments, the individualized cancer vaccine is administered intravenously, for example, wherein the RNA vaccine is administered to the human patient at doses of 15 μg, 21 μg, 21.3 μg, 25 μg, 38 μg, or 50 μg. In some embodiments, 15 μg, 21 μg, 21.3 μg, 25 μg, 38 μg, or 50 μg of RNA is delivered per dose (i.e., dose weight reflects the weight of RNA administered, not the total weight of the formulation administered). In some embodiments, the RNA vaccine is administered to the human patient at a dose of about 25 μg. In some embodiments, the RNA vaccine is administered to the human patient at a dose of about 21 μg. In some embodiments, the RNA vaccine is administered to the human patient at a dose of about 21.3 μg. More than one individualized cancer vaccine may be administered to a subject, e.g., subject is administered one individualized cancer vaccine with a combination of neoepitopes and also administered a separate individualized cancer vaccine with a different combination of neoepitopes. In some embodiments, a first individualized cancer vaccine with five neoepitopes is administered in combination with a second individualized cancer vaccine with five alternative epitopes. In some embodiments, a first individualized cancer vaccine with ten neoepitopes is administered in combination with a second individualized cancer vaccine with ten alternative epitopes.

In some embodiments, the individualized cancer vaccine is administered such that it is delivered to the spleen. For example, the individualized cancer vaccine can be administered such that one or more antigen(s) (e.g., tumor-specific neo- antigens) are delivered to antigen presenting cells (e.g., in the spleen).

Any of the individualized cancer vaccines or RNA vaccines of the present disclosure may find use in the methods described herein. For example, in some embodiments, a PD-1 axis binding antagonist of the present disclosure is administered in combination with an individualized cancer vaccine (ICV), e.g., an RNA vaccine described herein.

Further provided herein are DNA molecules encoding any of the RNA vaccines of the present disclosure. For example, in some embodiments, a DNA molecule of the present disclosure comprises the general structure (in the 5′→3′ direction): (1) a polynucleotide sequence encoding a 5′ untranslated region (UTR); (2) a polynucleotide sequence encoding a secretory signal peptide; (3) a polynucleotide sequence encoding at least a portion of a transmembrane and cytoplasmic domain of a major histocompatibility complex (MHC) molecule; (4) a polynucleotide sequence encoding a 3′ UTR comprising: (a) a 3′ untranslated region of an Amino-Terminal Enhancer of Split (AES) mRNA or a fragment thereof; and (b) non-coding RNA of a mitochondrially encoded 12S RNA or a fragment thereof; and (5) a polynucleotide sequence encoding a poly(A) sequence. In some embodiments, a DNA molecule of the present disclosure comprises, in the 5′→3′ direction: the polynucleotide sequence GGCGAACTAGTATTCTTCTGGTCCCCACAGACTCAGAGAGAACCCGCCACCATGAGAGT GATGGCCCCCAGAACCCTGATCCTGCTGCTGTCTGGCGCCCTGGCCCTGACAGAGACATG GGCCGGAAGC (SEQ ID NO:40); and the polynucleotide sequence

(SEQ ID NO: 41)
ATCGTGGGAATTGTGGCAGGACTGGCAGTGCTGGCCGTGGTGGTGATCGG
AGCCGTGGTGGCTACCGTGATGTGCAGACGGAAGTCCAGCGGAGGCAAGG
GCGGCAGCTACAGCCAGGCCGCCAGCTCTGATAGCGCCCAGGGCAGCGAC
GTGTCACTGACAGCCTAGTAACTCGAGCTGGTACTGCATGCACGCAATGC
TAGCTGCCCCTTTCCCGTCCTGGGTACCCCGAGTCTCCCCCGACCTCGGG
TCCCAGGTATGCTCCCACCTCCACCTGCCCCACTCACCACCTCTGCTAGT
TCCAGACACCTCCCAAGCACGCAGCAATGCAGCTCAAAACGCTTAGCCTA
GCCACACCCCCACGGGAAACAGCAGTGATTAACCTTTAGCAATAAACGAA
AGTTTAACTAAGCTATACTAACCCCAGGGTTGGTCAATTTCGTGCCAGCC
ACACCGAGACCTGGTCCAGAGTCGCTAGCCGCGTCGCT.

In some embodiments, the DNA molecule further comprises, in the 5′→3′ direction: a polynucleotide sequence encoding an amino acid linker; and a polynucleotide sequence encoding a neoepitope. In some embodiments, the polynucleotide sequences encoding the amino acid linker and the neoepitope form a linker-neoepitope module (e.g., a continuous sequence in the 5′→3′ direction in the same open-reading frame). In some embodiments, the polynucleotide sequences forming the linker-neoepitope module are between the polynucleotide sequence encoding the secretory signal peptide and the polynucleotide sequence encoding the at least portion of the transmembrane and cytoplasmic domain of the MHC molecule, or between the sequences of SEQ ID NO:40 and SEQ ID NO: 41, in the 5′→3′ direction. In some embodiments, the DNA molecule comprises 2, 3, 4, 5, 6, 7, 8, 9, 10, 11, 12, 13, 14, 15, 16, 17, 18, 19, 20, 21, 22, 23, 24, 25, 26, 27, 28, 28, 29, or 30 linker-epitope modules, and each of the linker-epitope modules encodes a different neoepitope. In some embodiments, the DNA molecule comprises 2, 3, 4, 5, 6, 7, 8, 9, 10, 11, 12, 13, 14, 15, 16, 17, 18, 19, or 20 linker-epitope modules, and the DNA molecule comprises polynucleotides encoding at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or 20 different neoepitopes. In some embodiments, the DNA molecule comprises 5, 10, or 20 linker-epitope modules. In some embodiments, each of the linker-epitope modules encodes a different neoepitope. In some embodiments, the linker-epitope modules form a continuous sequence in the 5′→3′ direction in the same open-reading frame. In some embodiments, the polynucleotide sequence encoding the linker of the first linker-epitope module is 3′ of the polynucleotide sequence encoding the secretory signal peptide. In some embodiments, the polynucleotide sequence encoding the neoepitope of the last linker-epitope module is 5′ of the polynucleotide sequence encoding the at least portion of the transmembrane and cytoplasmic domain of the MHC molecule.

Also provided herein are methods of producing any of the RNA vaccine of the present disclosure, comprising transcribing (e.g., by transcription of linear, double-stranded DNA or plasmid DNA, such as by in vitro transcription) a DNA molecule of the present disclosure. In some embodiments, the methods further comprise isolating and/or purifying the transcribed RNA molecule from the DNA molecule.

In some embodiments, an RNA or DNA molecule of the present disclosure comprises a type IIS restriction cleavage site, which allows RNA to be transcribed under the control of a 5′ RNA polymerase promoter and which contains a polyadenyl cassette (poly(A) sequence), wherein the recognition sequence is located 3′ of the poly(A) sequence, while the cleavage site is located upstream and thus within the poly(A) sequence. Restriction cleavage at the type IIS restriction cleavage site enables a plasmid to be linearized within the poly(A) sequence, as described in U.S. Pat. Nos. 9,476,055 and 10,106,800. The linearized plasmid can then be used as template for in vitro transcription, the resulting transcript ending in an unmasked poly(A) sequence. Any of the type IIS restriction cleavage sites described in U.S. Pat. Nos. 9,476,055 and 10,106,800 may be used.

In some embodiments of the methods provided herein, the RNA vaccine includes one or more polynucleotides encoding 5-20 (e.g., any of 5, 6, 7, 8, 9, 10, 11, 12, 13, 14, 15, 16, 17, 18, 19, or 20) neoepitopes resulting from cancer-specific somatic mutations present in the tumor specimen. In certain embodiments, the RNA vaccine is formulated with one or more lipids. In certain embodiments, the one or more polynucleotides of the RNA vaccine and the one or more lipids form a lipoplex. In certain embodiments, the lipoplex includes one or more lipids that form a multilamellar structure that encapsulates the one or more polynucleotides of the RNA vaccine. In certain embodiments, the one or more lipids include at least one cationic lipid and at least one helper lipid. In certain embodiments, the one or more lipids include
(R)-N,N,N-trimethyl-2,3-dioleyloxy-1-propanaminium chloride (DOTMA) and 1,2-dioleoyl-sn-glycero-3-phosphoethanolamine (DOPE). In certain embodiments, at physiological pH the overall charge ratio of positive charges to negative charges of the liposome is 1.3:2 (0.65).

In certain embodiments, the RNA vaccine includes an RNA molecule including, in the 5′→3′ direction: (1) a 5′ cap; (2) a 5′ untranslated region (UTR); (3) a polynucleotide sequence encoding a secretory signal peptide; (4) a polynucleotide sequence encoding the one or more neoepitopes resulting from cancer-specific somatic mutations present in the tumor specimen; (5) a polynucleotide sequence encoding at least a portion of a transmembrane and cytoplasmic domain of a major histocompatibility complex (MHC) molecule; (6) a 3′ UTR including: (a) a 3′ untranslated region of an Amino-Terminal Enhancer of Split (AES) mRNA or a fragment thereof; and (b) non-coding RNA of a mitochondrially encoded 12S RNA or a fragment thereof; and (7) a poly(A) sequence.

In certain embodiments, the RNA molecule further includes a polynucleotide sequence encoding an amino acid linker; wherein the polynucleotide sequences encoding the amino acid linker and a first of the one or more neoepitopes form a first linker-neoepitope module; and wherein the polynucleotide sequences forming the first linker-neoepitope module are between the polynucleotide sequence encoding the secretory signal peptide and the polynucleotide sequence encoding the at least portion of the transmembrane and cytoplasmic domain of the MHC molecule in the 5′→3′ direction. In certain embodiments, the amino acid linker includes the sequence GGSGGGGSGG (SEQ ID NO: 39). In certain embodiments, the polynucleotide sequence encoding the amino acid linker includes the sequence GGCGGCUCUGGAGGAGGCGGCUCCGGAGGC (SEQ ID NO: 37).

In certain embodiments, the RNA molecule further includes, in the 5′→3′ direction: at least a second linker-epitope module, wherein the at least second linker-epitope module includes a polynucleotide sequence encoding an amino acid linker and a polynucleotide sequence encoding a neoepitope; wherein the polynucleotide sequences forming the second linker-neoepitope module are between the polynucleotide sequence encoding the neoepitope of the first linker-neoepitope module and the polynucleotide sequence encoding the at least portion of the transmembrane and cytoplasmic domain of the MHC molecule in the 5′→3′ direction; and wherein the neoepitope of the first linker-epitope module is different from the neoepitope of the second linker-epitope module. In certain embodiments, the RNA molecule includes 5 linker-epitope modules, wherein the 5 linker-epitope modules each encode a different neoepitope. In certain embodiments, the RNA molecule includes 5 linker-epitope modules, wherein the 5 linker-epitope modules each encode a different neoepitope. In certain embodiments, the RNA molecule includes 10 linker-epitope modules, wherein the 10 linker-epitope modules each encode a different neoepitope. In certain embodiments, the RNA molecule includes 20 linker-epitope modules, wherein the 20 linker-epitope modules each encode a different neoepitope.

In certain embodiments, the RNA molecule further includes a second polynucleotide sequence encoding an amino acid linker, wherein the second polynucleotide sequence encoding the amino acid linker is between the polynucleotide sequence encoding the neoepitope that is most distal in the 3′ direction and the polynucleotide sequence encoding the at least portion of the transmembrane and cytoplasmic domain of the MHC molecule.

In certain embodiments, the 5′ cap includes a D1 diastereoisomer of the structure:

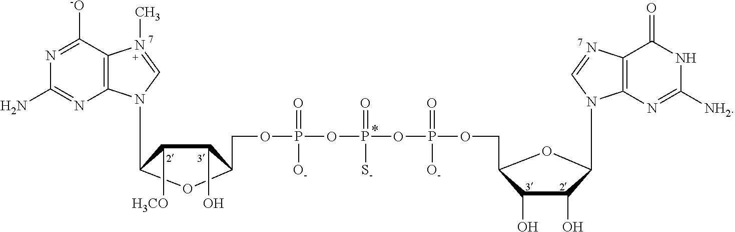

In certain embodiments, the 5′ UTR includes the sequence UUCUUCUGGUCCCCACAGACUCAGAGAGAACCCGCCACC (SEQ ID NO:23). In certain embodiments, the 5′ UTR includes the sequence

(SEQ ID NO: 21)
GGCGAACUAGUAUUCUUCUGGUCCCCACAGACUCAGAGAGAACCCGCCAC
C.

In certain embodiments, the secretory signal peptide includes the amino acid sequence MRVMAPRTLILLLSGALALTETWAGS (SEQ ID NO:27). In certain embodiments, the polynucleotide sequence encoding the secretory signal peptide includes the sequence

(SEQ ID NO: 25)
AUGAGAGUGAUGGCCCCCAGAACCCUGAUCCUGCUGCUGUCUGGCGCCCU
GGCCCUGACAGAGACAUGGGCCGGAAGC.

In certain embodiments, the at least portion of the transmembrane and cytoplasmic domain of the MHC molecule includes the amino acid sequence IVGIVAGLAVLAVVVIGAVVATVMCRRKSSGGKGGSYSQAASSDSAQGSDVSLTA (SEQ ID NO: 30). In certain embodiments, the polynucleotide sequence encoding the at least portion of the transmembrane and cytoplasmic domain of the MHC molecule includes the sequence

(SEQ ID NO: 28)
AUCGUGGGAAUUGUGGCAGGACUGGCAGUGCUGGCCGUGGUGGUGAUCGG
AGCCGUGGUGGCUACCGUGAUGUGCAGACGGAAGUCCAGCGGAGGCAAGG
GCGGCAGCUACAGCCAGGCCGCCAGCUCUGAUAGCGCCCAGGGCAGCGAC
GUGUCACUGACAGCC.

In certain embodiments, the 3′ untranslated region of the AES mRNA includes the sequence CUGGUACUGCAUGCACGCAAUGCUAGCUGCCCCUUUCCCGUCCUGGGUACCCCGAGUC UCCCCCGACCUCGGGUCCCAGGUAUGCUCCCACCUCCACCUGCCCCACUCACCACCUCU GCUAGUUCCAGACACCUCC (SEQ ID NO:33). In certain embodiments, the non-coding RNA of the mitochondrially encoded 12S RNA includes the sequence CAAGCACGCAGCAAUGCAGCUCAAAACGCUUAGCCUAGCCACACCCCCACGGGAAACA GCAGUGAUUAACCUUUAGCAAUAAACGAAAGUUUAACUAAGCUAUACUAACCCCAGG GUUGGUCAAUUUCGUGCCAGCCACACCG (SEQ ID NO:35). In certain embodiments, the 3′ UTR includes the sequence

(SEQ ID NO: 31)
CUCGAGCUGGUACUGCAUGCACGCAAUGCUAGCUGCCCCUUUCCCGUCCU
GGGUACCCCGAGUCUCCCCCGACCUCGGGUCCCAGGUAUGCUCCCACCUC
CACCUGCCCCACUCACCACCUCUGCUAGUUCCAGACACCUCCCAAGCACG
CAGCAAUGCAGCUCAAAACGCUUAGCCUAGCCACACCCCCACGGGAAACA
GCAGUGAUUAACCUUUAGCAAUAAACGAAAGUUUAACUAAGCUAUACUAA
CCCCAGGGUUGGUCAAUUUCGUGCCAGCCACACCGAGACCUGGUCCAGAG
UCGCUAGCCGCGUCGCU.

In certain embodiments, the poly(A) sequence includes 120 adenine nucleotides.

In certain embodiments, the RNA vaccine includes an RNA molecule including, in the 5′→3′ direction: the polynucleotide sequence GGCGAACUAGUAUUCUUCUGGUCCCCACAGACUCAGAGAGAACCCGCCACCAUGAGAG UGAUGGCCCCCAGAACCCUGAUCCUGCUGCUGUCUGGCGCCCUGGCCCUGACAGAGAC AUGGGCCGGAAGC (SEQ ID NO: 19); a polynucleotide sequence encoding the one or more neoepitopes resulting from cancer-specific somatic mutations present in the tumor specimen; and the polynucleotide sequence

(SEQ ID NO: 20)
AUCGUGGGAAUUGUGGCAGGACUGGCAGUGCUGGCCGUGGUGGUGAUCGG
AGCCGUGGUGGCUACCGUGAUGUGCAGACGGAAGUCCAGCGGAGGCAAGG
GCGGCAGCUACAGCCAGGCCGCCAGCUCUGAUAGCGCCCAGGGCAGCGAC
GUGUCACUGACAGCCUAGUAACUCGAGCUGGUACUGCAUGCACGCAAUGC
UAGCUGCCCCUUUCCCGUCCUGGGUACCCCGAGUCUCCCCCGACCUCGGG
UCCCAGGUAUGCUCCCACCUCCACCUGCCCCACUCACCACCUCUGCUAGU
UCCAGACACCUCCCAAGCACGCAGCAAUGCAGCUCAAAACGCUUAGCCUA
GCCACACCCCCACGGGAAACAGCAGUGAUUAACCUUUAGCAAUAAACGAA
AGUUUAACUAAGCUAUACUAACCCCAGGGUUGGUCAAUUUCGUGCCAGCC
ACACCGAGACCUGGUCCAGAGUCGCUAGCCGCGUCGCU.

### IV. PD-1 Axis Binding Antagonists

In some embodiments, an individualized cancer vaccine (e.g., an RNA vaccine) of the present disclosure is administered in combination with a PD-1 axis binding antagonist.

For example, a PD-1 axis binding antagonist includes a PD-1 binding antagonist, a PDL1 binding antagonist and a PDL2 binding antagonist. Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 binding antagonist is a molecule that inhibits the binding of PD-1 to its ligand binding partner(s). In a specific aspect, the PD-1 ligand binding partners are PDL1 and/or PDL2. In another embodiment, a PDL1 binding antagonist is a molecule that inhibits the binding of PDL1 to its binding partner(s). In a specific aspect, PDL1 binding partner(s) are PD-1 and/or B7-1. In another embodiment, the PDL2 binding antagonist is a molecule that inhibits the binding of PDL2 to its binding partner(s). In a specific aspect, a PDL2 binding partner is PD-1. The antagonist may be an antibody, an antigen binding fragment thereof, an immunoadhesin, a fusion protein, or oligopeptide.

In some embodiments, the PD-1 binding antagonist is an anti-PD-1 antibody (e.g., a human antibody, a humanized antibody, or a chimeric antibody).

In some embodiments, the anti-PD-1 antibody is nivolumab (CAS Registry Number: 946414-94-4). Nivolumab (Bristol-Myers Squibb/Ono), also known as MDX-1106-04, MDX-1106, ONO-4538, BMS-936558, and OPDIVO®, is an anti-PD-1 antibody described in WO2006/121168. In some embodiments, the anti-PD-1 antibody comprises a heavy chain and a light chain sequence, wherein:

(a) the heavy chain comprises the amino
acid sequence:
(SEQ ID NO: 11)
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSSASTKGPSVFPLAPCSRSTSESTAALGCLVKDYFPEPV
TVSWNSGALTSGVHTFPAVLQSSGLYSLSSVVTVPSSSLGTKTYTCNVDH
KPSNTKVDKRVESKYGPPCPPCPAPEFLGGPSVFLFPPKPKDTLMISRTP
EVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLY
SRLTVDKSRWQEGNVFSCSVMHEALHNHYTQKSLSLSLG,
and
(b) the light chain comprises the amino
acid sequence:
(SEQ ID NO: 12)
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In some embodiments, the anti-PD-1 antibody comprises the six HVR sequences from SEQ ID NO: 11 and SEQ ID NO: 12 (e.g., the three heavy chain HVRs from SEQ ID NO: 11 and the three light chain HVRs from SEQ ID NO: 12). In some embodiments, the anti-PD-1 antibody comprises the heavy chain variable domain from SEQ ID NO: 11 and the light chain variable domain from SEQ ID NO: 12.

In some embodiments, the anti-PD-1 antibody is pembrolizumab (CAS Registry Number: 1374853-91-4). Pembrolizumab (Merck), also known as MK-3475, Merck 3475, lambrolizumab, KEYTRUDA®, and SCH-900475, is an anti-PD-1 antibody described in WO2009/114335. In some embodiments, the anti-PD-1 antibody comprises a heavy chain and a light chain sequence, wherein:

(a) the heavy chain comprises the amino
acid sequence:
(SEQ ID NO: 13)
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLG,
and
(b) the light chain comprises the amino
acid sequence:
(SEQ ID NO: 14)
EIVLTQSPATLSLSPGERATLSCRASKGVSTSGYSYLHWYQQKPGQAPRL
LIYLASYLESGVPARFSGSGSGTDFTLTISSLEPEDFAVYYCQHSRDLPL
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC.

In some embodiments, the anti-PD-1 antibody comprises the six HVR sequences from SEQ ID NO: 13 and SEQ ID NO:14 (e.g., the three heavy chain HVRs from SEQ ID NO: 13 and the three light chain HVRs from SEQ ID NO:14). In some embodiments, the anti-PD-1 antibody comprises the heavy chain variable domain from SEQ ID NO:13 and the light chain variable domain from SEQ ID NO: 14.

In some embodiments, the anti-PD-1 antibody is MEDI-0680 (AMP-514; AstraZeneca). MEDI-0680 is a humanized IgG4 anti-PD-1 antibody.

In some embodiments, the anti-PD-1 antibody is PDR001 (CAS Registry No. 1859072-53-9; Novartis). PDR001 is a humanized IgG4 anti-PD1 antibody that blocks the binding of PDL1 and PDL2 to PD-1.

In some embodiments, the anti-PD-1 antibody is REGN2810 (Regeneron). REGN2810 is a human anti-PD1 antibody also known as LIBTAYO® and cemiplimab-rwlc.

In some embodiments, the anti-PD-1 antibody is BGB-108 (BeiGene). In some embodiments, the anti-PD-1 antibody is BGB-A317 (BeiGene).

In some embodiments, the anti-PD-1 antibody is JS-001 (Shanghai Junshi). JS-001 is a humanized anti-PD1 antibody.

In some embodiments, the anti-PD-1 antibody is STI-A1110 (Sorrento). STI-A1110 is a human anti-PD1 antibody.

In some embodiments, the anti-PD-1 antibody is INCSHR-1210 (Incyte). INCSHR-1210 is a human IgG4 anti-PD1 antibody.

In some embodiments, the anti-PD-1 antibody is PF-06801591 (Pfizer).

In some embodiments, the anti-PD-1 antibody is TSR-042 (also known as ANB011; Tesaro/AnaptysBio).

In some embodiments, the anti-PD-1 antibody is AM0001 (ARMO Biosciences).

In some embodiments, the anti-PD-1 antibody is ENUM 244C8 (Enumeral Biomedical Holdings). ENUM 244C8 is an anti-PD1 antibody that inhibits PD-1 function without blocking binding of PDL1 to PD-1.

In some embodiments, the anti-PD-1 antibody is ENUM 388D4 (Enumeral Biomedical Holdings). ENUM 388D4 is an anti-PD1 antibody that competitively inhibits binding of PDL1 to PD-1.

In some embodiments, the PD-1 antibody comprises the six HVR sequences (e.g., the three heavy chain HVRs and the three light chain HVRs) and/or the heavy chain variable domain and light chain variable domain from a PD-1 antibody described in WO2015/112800 (Applicant: Regeneron), WO2015/112805 (Applicant: Regeneron), WO2015/112900 (Applicant: Novartis), US20150210769 (Assigned to Novartis), WO2016/089873 (Applicant: Celgene), WO2015/035606 (Applicant: Beigene), WO2015/085847 (Applicants: Shanghai Hengrui Pharmaceutical/Jiangsu Hengrui Medicine), WO2014/206107 (Applicants: Shanghai Junshi Biosciences/Junmeng Biosciences), WO2012/145493 (Applicant: Amplimmune), U.S. Pat. No. 9,205,148 (Assigned to MedImmune), WO2015/119930 (Applicants: Pfizer/Merck), WO2015/119923 (Applicants: Pfizer/Merck), WO2016/032927 (Applicants: Pfizer/Merck), WO2014/179664 (Applicant: AnaptysBio), WO2016/106160 (Applicant: Enumeral), and WO2014/194302 (Applicant: Sorrento).

In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. AMP-224 (CAS Registry No. 1422184-00-6; GlaxoSmithKline/MedImmune), also known as B7-DCIg, is a PDL2-Fc fusion soluble receptor described in WO2010/027827 and WO2011/066342.

In some embodiments, the PD-1 binding antagonist is a peptide or small molecule compound. In some embodiments, the PD-1 binding antagonist is AUNP-12 (PierreFabre/Aurigene). See, e.g., WO2012/168944, WO2015/036927, WO2015/044900, WO2015/033303, WO2013/144704, WO2013/132317, and WO2011/161699.

In some embodiments, the PDLI binding antagonist is a small molecule that inhibits PD-1. In some embodiments, the PDL1 binding antagonist is a small molecule that inhibits PDL1. In some embodiments, the PDL1 binding antagonist is a small molecule that inhibits PDL1 and VISTA. In some embodiments, the PDL1 binding antagonist is CA-170 (also known as AUPM-170). In some embodiments, the PDL1 binding antagonist is a small molecule that inhibits PDL1 and TIM3. In some embodiments, the small molecule is a compound described in WO2015/033301 and WO2015/033299.

In some embodiments, the PD-1 axis binding antagonist is an anti-PDL1 antibody. A variety of anti-PDL1 antibodies are contemplated and described herein. In any of the embodiments herein, the isolated anti-PDL1 antibody can bind to a human PDL1, for example a human PDL1 as shown in UniProtKB/Swiss-Prot Accession No. Q9NZQ7.1, or a variant thereof. In some embodiments, the anti-PDL1 antibody is capable of inhibiting binding between PDL1 and PD-1 and/or between PDL1 and B7-1. In some embodiments, the anti-PDL1 antibody is a monoclonal antibody. In some embodiments, the anti-PDL1 antibody is an antibody fragment selected from the group consisting of Fab, Fab′-SH, Fv, scFv, and (Fab′) 2 fragments. In some embodiments, the anti-PDLI antibody is a humanized antibody. In some embodiments, the anti-PDL1 antibody is a human antibody. Examples of anti-PDL1 antibodies useful for the methods of this invention, and methods for making thereof are described in PCT patent application WO 2010/077634 A1 and U.S. Pat. No. 8,217,149, which are incorporated herein by reference.

In some embodiments, the anti-PDL1 antibody comprises a heavy chain variable region and a light chain variable region, wherein:
(a) the heavy chain variable region comprises an HVR-H1, HVR-H2, and HVR-H3 sequence of GFTFSDSWIH (SEQ ID NO:1), AWISPYGGSTYYADSVKG (SEQ ID NO:2) and RHWPGGFDY (SEQ ID NO:3), respectively, and
(b) the light chain variable region comprises an HVR-L1, HVR-L2, and HVR-L3 sequence of RASQDVSTAVA (SEQ ID NO:4), SASFLYS (SEQ ID NO:5) and QQYLYHPAT (SEQ ID NO: 6), respectively.

In some embodiments, the anti-PDL1 antibody is MPDL3280A, also known as atezolizumab and TECENTRIQ® (CAS Registry Number: 1422185 Jun. 5), with a WHO Drug Information (International Nonproprietary Names for Pharmaceutical Substances), Proposed INN: List 112, Vol. 28, No. 4, published Jan. 16, 2015 (see page 485) described therein. In some embodiments, the anti-PDL1 antibody comprises a heavy chain and a light chain sequence, wherein:

(a) the heavy chain variable region sequence
comprises the amino acid sequence:
(SEQ ID NO: 7)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSS,
and
(b) the light chain variable region sequence
comprises the amino acid sequence:
(SEQ ID NO: 8)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKR.

In some embodiments, the anti-PDL1 antibody comprises a heavy chain and a light chain sequence, wherein:

(a) the heavy chain comprises the amino acid sequence:
(SEQ ID NO: 9)
EVQLVESGGGLVQPGGSLRLSCAASGFTFSDSWIHWVRQAPGKGLEWVAWISPYGGSTYYA
DSVKGRFTISADTSKNTAYLQMNSLRAEDTAVYYCARRHWPGGFDYWGQGTLVTVSSAST
KGPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYASTYRVVSVLTV
LHQDWLNGKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHEAL
HNHYTQKSLSLSPG,
and
(b) the light chain comprises the amino acid sequence:
(SEQ ID NO: 10)
DIQMTQSPSSLSASVGDRVTITCRASQDVSTAVAWYQQKPGKAPKLLIYSASFLYSGVPSRFS
GSGSGTDFTLTISSLQPEDFATYYCQQYLYHPATFGQGTKVEIKRTVAAPSVFIFPPSDEQLKS
GTASVVCLLNNFYPREAKVQWKVDNALQSGNSQESVTEQDSKDSTYSLSSTLTLSKADYEK
HKVYACEVTHQGLSSPVTKSFNRGEC.

In some embodiments, the anti-PDL1 antibody is avelumab (CAS Registry Number: 1537032-82-8). Avelumab, also known as MSB0010718C, is a human monoclonal IgGI anti-PDL1 antibody (Merck KGaA, Pfizer). In some embodiments, the anti-PDL1 antibody comprises a heavy chain and a light chain sequence, wherein:

(a) the heavy chain comprises the amino acid sequence:
(SEQ ID NO: 15)
EVQLLESGGGLVQPGGSLRLSCAASGFTFSSYIMMWVRQAPGKGLEWVSSIYPSGGITFYAD
TVKGRFTISRDNSKNTLYLQMNSLRAEDTAVYYCARIKLGTVTTVDYWGQGTLVTVSSAST
KGPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEAL
HNHYTQKSLSLSPG,
and
(b) the light chain comprises the amino acid sequence:
(SEQ ID NO: 16)
QSALTQPASVSGSPGQSITISCTGTSSDVGGYNYVSWYQQHPGKAPKLMIYDVSNRPSGVSN
RFSGSKSGNTASLTISGLQAEDEADYYCSSYTSSSTRVFGTGTKVTVLGQPKANPTVTLFPPSS
EELQANKATLVCLISDFYPGAVTVAWKADGSPVKAGVETTKPSKQSNNKYAASSYLSLTPEQ
WKSHRSYSCQVTHEGSTVEKTVAPTECS.

In some embodiments, the anti-PDL1 antibody comprises the six HVR sequences from SEQ ID NO:15 and SEQ ID NO:16 (e.g., the three heavy chain HVRs from SEQ ID NO: 15 and the three light chain HVRs from SEQ ID NO:16). In some embodiments, the anti-PDL1 antibody comprises the heavy chain variable domain from SEQ ID NO: 15 and the light chain variable domain from SEQ ID NO:16.

In some embodiments, the anti-PDL1 antibody is durvalumab (CAS Registry Number: 1428935-60-7). Durvalumab, also known as MEDI4736, is an Fc optimized human monoclonal IgG1 kappa anti-PDL1 antibody (MedImmune, AstraZeneca) described in WO2011/066389 and US2013/034559. In some embodiments, the anti-PDL1 antibody comprises a heavy chain and a light chain sequence, wherein:

(a) the heavy chain comprises the amino acid sequence:
(SEQ ID NO: 17)
EVQLVESGGGLVQPGGSLRLSCAASGFTFSRYWMSWVRQAPGKGLEWVANIKQDGSEKYY
VDSVKGRFTISRDNAKNSLYLQMNSLRAEDTAVYYCAREGGWFGELAFDYWGQGTLVTVS
SASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPCPAPEFEGGPSVFLF
PPKPKDTLMISRTPEVTCVVVDVSHEDPEVKFNWYVDGVEVHNAKTKPREEQYNSTYRVVS
VLTVLHQDWLNGKEYKCKVSNKALPASIEKTISKAKGQPREPQVYTLPPSREEMTKNQVSLT
CLVKGFYPSDIAVEWESNGQPENNYKTTPPVLDSDGSFFLYSKLTVDKSRWQQGNVFSCSV
MHEALHNHYTQKSLSLSPG,
and
(b) the light chain comprises the amino acid sequence:
(SEQ ID NO: 18)
EIVLTQSPGTLSLSPGERATLSCRASQRVSSSYLAWYQQKPGQAPRLLIYDASSRATGIPDRFS
GSGSGTDFTLTISRLEPEDFAVYYCQQYGSLPWTFGQGTKVEIKRTVAAPSVFIFPPSDEQLKS
GTASVVCLLNNFYPREAKVQWKVDNALQSGNSQESVTEQDSKDSTYSLSSTLTLSKADYEK
HKVYACEVTHQGLSSPVTKSFNRGEC.

In some embodiments, the anti-PDL1 antibody comprises the six HVR sequences from SEQ ID NO: 17 and SEQ ID NO: 18 (e.g., the three heavy chain HVRs from SEQ ID NO: 17 and the three light chain HVRs from SEQ ID NO:18). In some embodiments, the anti-PDL1 antibody comprises the heavy chain variable domain from SEQ ID NO: 17 and the light chain variable domain from SEQ ID NO:18.

In some embodiments, the anti-PDL1 antibody is MDX-1105 (Bristol Myers Squibb). MDX-1105, also known as BMS-936559, is an anti-PDL1 antibody described in WO2007/005874.

In some embodiments, the anti-PDL1 antibody is LY3300054 (Eli Lilly).

In some embodiments, the anti-PDL1 antibody is STI-A1014 (Sorrento). STI-A1014 is a human anti-PDL1 antibody.

In some embodiments, the anti-PDL1 antibody is KN035 (Suzhou Alphamab). KN035 is single-domain antibody (dAB) generated from a camel phage display library.

In some embodiments, the anti-PDL1 antibody comprises a cleavable moiety or linker that, when cleaved (e.g., by a protease in the tumor microenvironment), activates an antibody antigen binding domain to allow it to bind its antigen, e.g., by removing a non-binding steric moiety. In some embodiments, the anti-PDL1 antibody is CX-072 (CytomX Therapeutics).

In some embodiments, the PDLI antibody comprises the six HVR sequences (e.g., the three heavy chain HVRs and the three light chain HVRs) and/or the heavy chain variable domain and light chain variable domain from a PDL1 antibody described in US20160108123 (Assigned to Novartis), WO2016/000619 (Applicant: Beigene), WO2012/145493 (Applicant: Amplimmune), U.S. Pat. No. 9,205,148 (Assigned to MedImmune), WO2013/181634 (Applicant: Sorrento), and WO2016/061142 (Applicant: Novartis).

In a still further specific aspect, the antibody further comprises a human or murine constant region. In a still further aspect, the human constant region is selected from the group consisting of IgG1, IgG2, IgG2, IgG3, IgG4. In a still further specific aspect, the human constant region is IgG1. In a still further aspect, the murine constant region is selected from the group consisting of IgG1, IgG2A, IgG2B, IgG3. In a still further aspect, the murine constant region if IgG2A.

In a still further specific aspect, the antibody has reduced or minimal effector function. In a still further specific aspect, the minimal effector function results from an “effector-less Fc mutation” or aglycosylation mutation. In still a further embodiment, the effector-less Fc mutation is an N297A or D265A/N297A substitution in the constant region. In some embodiments, the isolated anti-PDL1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Removal of glycosylation sites form an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site another amino acid residue (e.g., glycine, alanine or a conservative substitution).

In a still further embodiment, the present disclosure provides for compositions comprising any of the above described anti-PDL1 antibodies in combination with at least one pharmaceutically-acceptable carrier.

In a still further embodiment, the present disclosure provides for a composition comprising an anti-PDL1, an anti-PD-1, or an anti-PDL2 antibody or antigen binding fragment thereof as provided herein and at least one pharmaceutically acceptable carrier. In some embodiments, the anti-PDL1, anti-PD-1, or anti-PDL2 antibody or antigen binding fragment thereof administered to the individual is a composition comprising one or more pharmaceutically acceptable carrier. Any of the pharmaceutically acceptable carriers described herein or known in the art may be used.

In some embodiments, the PD-1 axis binding antagonist is administered intravenously to the human patient. In some embodiments, the anti-PD-L1 antibody is administered to the human patient at a dose of about 1200 mg or about 1680 mg, for example about any of 1100 mg, 1150 mg, 1200 mg, 1250 mg, or 1300 mg or about any of 1600 mg, 1610 mg, 1620 mg, 1630 mg, 1640 mg, 1650 mg, 1660 mg, 1670 me, 1680 mg, 1690 mg, 1700 mg, or more. In some embodiments, the anti-PD-L1 antibody is atezolizumab, and the atezolizumab is administered intravenously to the human patient at a dose of about 1680 mg.

### V. Chemotherapy Treatments

In some embodiments, an individualized cancer vaccine (e.g., an RNA vaccine) of the present disclosure is administered in combination with a PD-1 axis binding antagonist and a chemotherapy treatment.

For example, the chemotherapeutic treatment may comprise a chemotherapeutic agent. Examples of chemotherapeutic agents may include but are not limited to any of gemcitabine, leucovorin, 5-fluorouracil, capecitabine, irinotecan, liposomal irinotecan, a platinum-based chemotherapeutic agent, an auristatin, a vinca alkaloid, a podophyllotoxin, a taxane, a baccatin derivative, a cryptophysin, a maytansinoid, a combretastatin, and a dolastatin. In some embodiments, the chemotherapeutic treatment comprises one or more of gemcitabine, leucovorin, 5-fluorouracil, capecitabine, irinotecan, liposomal irinotecan, a platinum-based chemotherapeutic agent, a taxane, and any combination thereof. Further examples of chemotherapeutic agents include an auristatin, a DNA minor groove binding agent, a DNA minor groove alkylating agent, an enediyne, a lexitropsin, a duocarmycin, a taxane, a puromycin, a dolastatin, a maytansinoid, and a vinca alkaloid.

In some embodiments, the platinum-based chemotherapeutic agent is cisplatin, oxaliplatin, or both. Cisplatin, also known as Platinol® and Platinol®-AQ, is an antineoplastic alkylating agent. Oxaliplatin, also known as Eloxatin, also is an antineoplastic alkylating agent.

In some embodiments, the taxane is paclitaxel, docetaxel, albumin-bound paclitaxel, or any combination thereof. Taxanes are antimicrotubule agents that act to stall the process of mitosis, thereby preventing cancer cells from dividing and proliferating.

Standard of care chemotherapy treatments for patients after PDAC resection involves adjuvant therapy with gemcitabine combination therapy or mFOLFIRINOX. Gemcitabine monotherapy had been a standard of care first-line treatment for advanced pancreatic cancer for more than 20 years. However, it was recently identified that combination chemotherapy regimens (e.g., FOLFIRINOX or gecitabine/nab-paclitaxel) achieved higher response rates and better overall survival than gemcitabine monotherapy. These combination therapies have become standard of care first-line treatments for advanced pancreatic cancer and available as a treatment option for borderline resectable pancreatic cancer and also for locally advanced pancreatic cancer (see, e.g., Saung, M.T. and Zheng, L., Clin Ther; 39 (11): 2125-2134 (2017)).

In some embodiments, the chemotherapeutic treatment comprises leucovorin, 5-fluorouracil, irinotecan, and oxaliplatin. The combination treatment with leucovorin, 5-fluorouracil, irinotecan, and oxaliplatin is also known as FOLFIRINOX. In some embodiments, the chemotherapeutic treatment is a FOLFIRINOX treatment or a modified FOLFIRINOX (mFOLFIRINOX) treatment. The modified treatment regimen in mFOLFIRINOX can be selected in order to decrease the incidence and severity of hematologic toxic effects and diarrhea without decreasing treatment efficacy. In some embodiments, the chemotherapeutic treatment comprises: oxaliplatin at a dose of about 85 mg/m²; leucovorin at a dose of about 400 mg/m²; irinotecan at a dose of about 150 mg/m²; and/or 5-fluorouracil at a dose of about 2400 mg/m². In some embodiments, the chemotherapeutic treatment is administered intravenously to the human patient. In some embodiments, the chemotherapeutic treatment is administered as described herein.

### VI. Pharmaceutical Compositions and Formulations

Also provided herein are pharmaceutical compositions and formulations, e.g., for the treatment of pancreatic cancer. In some embodiments, the pharmaceutical compositions and formulations further comprise a pharmaceutically acceptable carrier.

After preparation of the antibody of interest (e.g., techniques for producing antibodies which can be formulated as disclosed herein are elaborated herein and are known in the art), the pharmaceutical formulation comprising said antibody is prepared. In certain embodiments, the antibody to be formulated has not been subjected to prior lyophilization, and the formulation of interest herein is an aqueous formulation. In certain embodiments, the antibody is a full-length antibody. In one embodiment, the antibody in the formulation is an antibody fragment, such as an F (ab′) 2. The therapeutically effective amount of antibody present in the formulation is determined by taking into account the desired dose volumes and mode(s) of administration, for example. From about 25 mg/mL to about 150 mg/mL, or from about 30 mg/mL to about 140 mg/mL, or from about 35 mg/mL to about 130 mg/mL, or from about 40 mg/mL to about 120 mg/mL, or from about 50 mg/mL to about 130 mg/mL, or from about 50 mg/mL to about 125 mg/mL, or from about 50 mg/mL to about 120 mg/mL, or from about 50 mg/mL to about 110 mg/mL, or from about 50 mg/mL to about 100 mg/mL, or from about 50 mg/mL to about 90 mg/mL, or from about 50 mg/mL to about 80 mg/mL, or from about 54 mg/mL to about 66 mg/mL is an exemplary antibody concentration in the formulation. In some embodiments, an anti-PDL1 antibody described herein (such as atezolizumab) is administered at a dose of about 1200 mg. In some embodiments, an anti-PD1 antibody described herein (such as pembrolizumab) is administered at a dose of about 200 mg. In some embodiments, an anti-PD1 antibody described herein (such as nivolumab) is administered at a dose of about 240 mg (e.g., every 2 weeks) or 480 mg (e.g., every 4 weeks).

In some embodiments, an RNA vaccine described herein is administered at a dose of about 15 μg, about 21 μg, about 21.3 μg, about 25 μg, about 38 μg, or about 50 μg. For example, in some embodiments, the RNA vaccine is administered to the human patient at a dose of about 21 μg, about 21.3 μg, or about 25 μg.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The composition and formulation herein may also contain more than one active ingredient as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g., films, or microcapsules. The formulations to be used for in vivo administration are generally sterile. Sterility may be readily accomplished, e.g., by filtration through sterile filtration membranes.

Pharmaceutical formulations of atezolizumab and pembrolizumab are commercially available. For example, atezolizumab is known under the trade name (as described elsewhere herein) TECENTRIQ®. Pembrolizumab is known under the trade name (as described elsewhere herein) KEYTRUDA®. In some embodiments, atezolizumab and the RNA vaccine, or pembrolizumab and the RNA vaccine, are provided in separate containers. In some embodiments, atezolizumab and pembrolizumab are used and/or prepared for administration to an individual as described in the prescribing information available with the commercially available product.

### VII. Articles of Manufacture or Kits

Further provided herein is an article of manufacture or kit comprising an RNA vaccine of the present disclosure. Further provided herein is an article of manufacture or a kit comprising a PD-1 axis binding antagonist (such as atezolizumab or pembrolizumab). In some embodiments, the article of manufacture or kit further comprises package insert comprising instructions for using the RNA vaccine and/or PD-1 axis binding antagonist (e.g., in conjunction with the RNA vaccine) to treat or delay progression of pancreatic cancer in an individual. Also provided herein is an article of manufacture or a kit comprising a PD-1 axis binding antagonist (such as atezolizumab or pembrolizumab) and an RNA vaccine.

In some embodiments, there is provided a kit comprising an individualized RNA vaccine, for use in a method for treating a pancreatic cancer tumor in a human in need thereof, wherein the RNA vaccine is to be administered in combination with a PD-1 axis binding antagonist and a chemotherapeutic treatment according to the method described herein, wherein the RNA vaccine comprises one or more polynucleotides encoding one or more neoepitopes resulting from cancer-specific somatic mutations present in a pancreatic cancer tumor specimen obtained from the human. In some embodiments, the kit comprises a PD-1 axis binding antagonist for use in a method for treating a pancreatic cancer tumor in a human in need thereof, wherein the PD-1 axis binding antagonist is to be administered in combination with an individualized RNA vaccine and a chemotherapeutic treatment according to the method described herein, wherein the RNA vaccine comprises one or more polynucleotides encoding one or more neoepitopes resulting from cancer-specific somatic mutations present in a pancreatic cancer tumor specimen obtained from the human.

In some embodiments, the PD-1 axis binding antagonist and the RNA vaccine are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agents include, for example, bottles, vials, bags and syringes.

### VIII. Methods of Patient Selection

Further provided herein is a method of selecting a patient (such as a human patient) having a cancer tumor as likely to respond to a therapy comprising an individualized RNA vaccine, wherein the method comprises: a) measuring the number and/or frequency of de novo significantly expanded (SE) TCR clones in a sample from the patient by T cell receptor sequencing; b) comparing the number and/or frequency of de novo SE TCR clones measured in a) to a reference number and/or frequency; and c) selecting the patient as more likely to respond to the therapy comprising the individualized RNA vaccine when the number and/or frequency of de novo SE TCR clones in the sample from the patient is above the reference number and/or frequency; wherein the individualized RNA vaccine comprises one or more polynucleotides encoding one or more neoepitopes resulting from cancer-specific somatic mutations present in a cancer tumor specimen obtained from the patient, and wherein a number and/or frequency of de novo SE TCR clones above the reference number and/or frequency indicates that the patient is more likely to respond to the therapy comprising the individualized RNA vaccine. In some embodiments, the method further comprises selecting the therapy comprising the individualized RNA vaccine or recommending the therapy comprising the individualized RNA vaccine.

Also provided herein is a method of selecting a human patient having a cancer tumor as likely to respond to a therapy comprising an individualized RNA vaccine, the method comprising: a) comparing the number and/or frequency of de novo significantly expanded (SE) TCR clones in a sample from the patient to a reference number and/or frequency; and b) selecting the patient as more likely to respond to the therapy comprising the individualized RNA vaccine when the number and/or frequency of de novo SE TCR clones in the sample from the patient is above the reference number and/or frequency; wherein the individualized RNA vaccine comprises one or more polynucleotides encoding one or more neoepitopes resulting from cancer-specific somatic mutations present in a cancer tumor specimen obtained from the patient, wherein the number and/or frequency of de novo SE TCR clones is measured by T cell receptor sequencing, and wherein a number and/or frequency of de novo SE TCR clones above the reference number and/or frequency indicates that the patient is more likely to respond to the therapy comprising the individualized RNA vaccine.

### De Novo Significantly Expanded (SE) TCR Clones

The present disclosure describes how de novo SE TCR clones can be quantified, analyzed, and used to determine the likelihood of therapeutic efficacy and immunogenic response to the individualized RNA vaccine described herein.

T cell receptor (TCR) clones are generated during TCR gene rearrangement, and clonality can be identified and characterized, for example to identify the presence of specific antigens in an individual e.g., by identifying clonal expansion of T cells post- antigen exposure. During the process of T cell maturation, CD4+ and CD8+ T cells undergo T cell receptor gene rearrangement of the TCR-α,-β,-γ, and -δ loci. Rearrangement of TCR genes involves the somatic splicing of the variable (V), joining (J), and, in the β but not a chain, diversity (D) regions of the genome in immature T cells. This process is responsible for the diversity in antigen binding regions of the T cell receptor. It is estimated that there are up to 10¹⁵ (see, e.g., Ndifon et al. 2012) to 10⁶¹ TCR clone possibilities (see, e.g., Mora & Walczak 2019). Upon successful survival and maturation, each of these T cells has the capacity to further proliferate, giving rise to populations of T cell clones, wherein each clonal population comprises the same TCR sequence, having originated from the same single T cell. If the TCR of a naïve T cell binds to a specific antigen (e.g., cancer neoantigen)-MHC complex with sufficient affinity, the T cell will clonally expand. This expansion significantly increases the abundance of T cells that recognize a specific pathogen (e.g., a cancer tumor neoepitope) and consequently, enables an effective immune response. As a result of unique V (D) J recombination occurring in each maturing T cell, the nucleotide and/or amino acid sequence of the TCR that is present on each T cell can serve as a natural molecular barcode to track the presence, number, and abundance (i.e., frequency) of T cell clones at various stages of treatment.

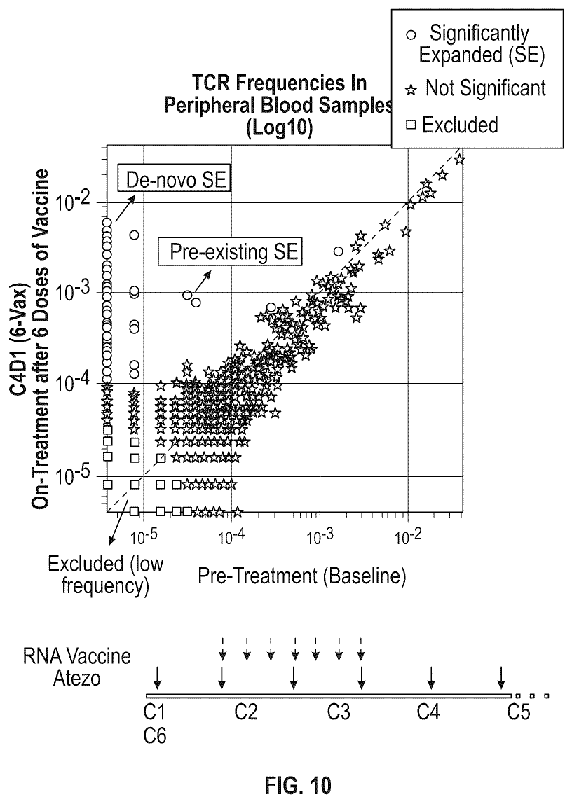

A significantly expanded (SE) TCR clone as used herein refers to a TCR clone that has expanded frequency (i.e., increased percentage or proportion as compared to total number of TCR clones) during and/or after treatment compared to baseline (i.e., compared to pre-treatment), as identified by methods such as TCR sequencing (“TCR-seq”). Statistical methods, e.g., binomial models based on a Fisher's exact test (DeWitt et al., J Virol 2015;89 (8): 4517-4526) or beta-binomial models (Rytlewski et al., PLOS One 2019; 14 (3): e0213684), can be applied to the data to identify which TCR clones show a statistically significant increase in clonal frequency during and/or after administration of the individualized RNA vaccine. In some embodiments, the frequency of TCR clones measured at a time point during or after treatment administration is compared to the frequency of TCR clones measured at pre-treatment baseline using a beta-binomial model. In some embodiments, the frequency of TCR clones measured at a time point during or after treatment administration is compared to the frequency of TCR clones measured at pre-treatment baseline using Fisher's exact test. In some embodiments, statistical corrections, such as a post-hoc correction, for example a Benjamini-Hochberg correction, is performed to control for false discovery rate. For example, see FIG. 10.

In some embodiments, the SE TCR clone is present in a sample at baseline. In some embodiments, the SE TCR clone is present at baseline at frequencies less than about any one of 10⁻³, 10⁻⁴, 10⁻⁵, 10⁻⁶, 10⁻⁷, 10⁻⁸, or lower frequency. In some embodiments, the SE TCR clone frequency is significantly greater after administration of treatment with the individualized RNA vaccine compared to pre-treatment baseline. Without wishing to be bound by theory, it is believed that these SE TCR clones are responsive to the individualized RNA vaccine but not responsive to the cancer tumor being treated such that the SE TCR clones did not (or minimally) clonally expand prior to administration of the individualized RNA vaccine targeting the cancer tumor.

In other embodiments, the SE TCR clone is a de novo SE TCR clone, and was not detected in the pre-treatment sample, i.e., the TCR clone arose after onset of administration of the individualized RNA vaccine, thereby indicating that the TCR clone arose specifically as a result of treatment.

Measuring Number and/or Frequency of De Novo SE TCR Clones

In the present disclosure, measurements of de novo SE TCR clones include the number and/or the frequency of de novo SE TCR clones. The number of TCR clones is the quantification of how many unique TCR clones are present in the sample. The frequency of TCR clones is a quantification of the abundance of T cells expressing a specific TCR clone.

As noted in the above section, the frequency of each TCR clone can be compared to TCR clones at pre-treatment baseline to identify which TCR clones are significantly expanded. Then, the TCR clones that significantly expanded, but which were not present prior to treatment can be identified and quantified to identify the number of de novo SE TCR clones. The number of de novo SE TCR clones can then be compared to a reference level as described in more detail below.

The method of quantifying the frequency of TCR clones is dependent upon the technology used to measure TCR clones, for example bulk TCR sequencing (bulk TCR-seq) or single cell TCR sequencing (scTCR-seq), as described below. In some instances, the frequency of TCR clones is calculated as a percentage or proportion of the T cells expressing a specific TCR clone divided by the total number of T cells in the sample). In other instances, the frequency of TCR clones is quantified as the number of mRNA reads for a specific TCR clone divided by the total number of TCR read counts in the sample. TCR clones with higher frequencies are representative of clonally expanded T cells, as described above.

The presence, number of TCR clones, and frequency of each TCR clone can be assessed using T cell receptor sequencing (TCR-seq), such as bulk TCR-seq or single cell TCR-seq (scTCR-seq), for example high-throughput sequencing such as (but not limited to) methods including the 10× Chromium Single Cell 5′ sequencing with V (D) J Enrichment or Single-Cell Immune Profiling. TCR-seq can identify the presence and sequence of each TCR clone present in a sample, e.g., a patient sample such as a cancer patient sample. Single cell TCR-seq provides both TCR messenger RNA (mRNA) expression and TCR clone frequencies in the same assay. In some embodiments, TCR clones are identified by nucleic acid sequencing, for example transcriptomic analyses. In some embodiments, TCR clones are identified by proteomic immune profiling analyses.

Samples containing T cells are collected from the individual, e.g., the patient, such as the cancer patient. Samples may be obtained from any tissue, organ, biopsy, or bodily fluid wherein T cells are present. For example, samples may be obtained from blood, lymph, bone marrow, spleen, lymph node(s) (such as lymph nodes peripheral to a tumor), cerebrospinal fluid, tonsils, tumor biopsy, etc. In some embodiments, the sample is obtained from the blood. In some embodiments, peripheral blood mononuclear cells (PBMC) are isolated from the blood sample. In some embodiments, T cells are enriched from isolated PBMC. In some embodiments, the enriched T cells are CD4+ T cells. In some embodiments, the enriched T cells are CD8+ T cells. In some embodiments, the enriched T cells are CD3+ T cells, wherein the CD3+ T cell population encompasses both CD4+ T cells and CD8+ T cells.

In some embodiments, the individual is a cancer patient, such as a human cancer patient. In some embodiments, the cancer is selected from the group consisting of non-small cell lung cancer (NSCLC), melanoma, renal cell carcinoma (RCC), breast cancer, colorectal cancer (CRC), ovarian cancer, prostate cancer, urinary bladder cancer (UBC), cervical cancer, bone cancer, head and neck squamous cell carcinoma (HNSCC), and pancreatic cancer. In some embodiments, the cancer tumor is a pancreatic cancer tumor. In some embodiments, the cancer tumor is a pancreatic ductal adenocarcinoma (PDAC) tumor. In some embodiments, at least five neoepitopes resulting from cancer-specific somatic mutations are present in a tumor specimen obtained from the human cancer patient prior to administration of an individualized RNA vaccine.

Samples can be obtained from an individual (e.g., a cancer patient) at any point during and/or after the treatment. A reference sample is obtained from the individual prior to administration of treatment for establishing baseline values, e.g., baseline number and/or frequency of TCR clones. Samples are then analyzed for the number and/or frequency of TCR clones, for example SE TCR clones, in particular de novo SE TCR clones, as described herein. In some embodiments, a sample is obtained after dose 1, dose 2, dose 3, dose 4, dose 5, dose 6, dose 7, dose 8, dose 9, dose 10 or more of an individualized RNA vaccine described in the present disclosure. In some embodiments, a sample is obtained after dose 2, dose 3, dose 6, dose 8, dose 9, and/or dose 10 of the individualized RNA vaccine. In some embodiments, a sample is obtained after administration of 6 doses of the individualized RNA vaccine.

In some embodiments, a sample is obtained after termination of treatment. In some embodiments, a sample is obtained after about any of 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 week, 2 weeks, 3 weeks, 4 weeks, 5 weeks, 6 weeks, 7 weeks, 8 weeks, 9 weeks, 10 weeks, 11 weeks, 12 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 1 year, 2 years, 3 years, 4 years, 5 years, 6 years, 7 years, 8 years, 9 years, 10 years, or more after termination of treatment.

In some embodiments, the number and/or frequency of TCR clones is measured prior to treatment initiation with the individualized cancer vaccine. In some embodiments, the number and/or frequency of de novo SE TCR clones is measured after any one or more of one dose, two doses, three doses, four doses, 5 doses, six doses, seven doses, eight doses, nine doses, ten doses, or more of the individualized cancer vaccine. In some embodiments, the number and/or frequency of de novo SE TCR clones is measured after six doses of the individualized cancer vaccine.

In some embodiments, the number and/or frequency of de novo SE TCR clones is measured after termination of treatment with the individualized cancer vaccine. In some embodiments, the number and/or frequency of de novo SE TCR clones is measured after about any of 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 week, 2 weeks, 3 weeks, 4 weeks, 5 weeks, 6 weeks, 7 weeks, 8 weeks, 9 weeks, 10 weeks, 11 weeks, 12 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 1 year, 2 years, 3 years, 4 years, 5 years, 6 years, 7 years, 8 years, 9 years, 10 years, or more after termination of treatment.

### Comparing the De Novo SE TCR Clone Measurement to a Reference Level

The number of T cell clones or clones and frequency of each T cell clone or clone in a patient treated with the individualized RNA vaccine of the present disclosure is compared to a reference value or level (e.g., a reference number or reference frequency). This comparison is performed to detect the patient's immune response or immunogenicity to the individualized RNA vaccine. In some embodiments, the reference level, e.g., the reference number or reference frequency, can be determined in a reference sample obtained from a patient prior to administration of an individualized RNA vaccine. In certain embodiments, a reference sample obtained from a patient prior to administration of an individualized RNA vaccine is a baseline sample. In certain embodiments, the reference level can be statistically calculated or set as determined from the overall distribution of the values in reference samples from cancer patients, e.g., pancreatic cancer patients, for example patients with pancreatic ductal adenocarcinoma.

A reference number and/or reference frequency of de novo SE TCR clones can define a cutoff whereby the number or frequency of de novo SE TCR clones above that cutoff, as determined by computational analysis, is correlated to an increased likelihood for the patient to display an immune response to the individualized RNA vaccine as a single agent or in combination with checkpoint blockade that is indicative of improved or otherwise successful cancer treatment, as defined in the present disclosure.

In one embodiment, the reference level is set as a cutoff value, such that the cutoff value is indicative of the value at which, e.g., the rate of predicting immunogenicity and/or immune response to the therapy described herein reaches a specificity of about 100% and a sensitivity of about 80%. In some embodiments, the specificity can reach about any of 90%, 91%, 92%, 93%, 94%, 95%, 96%, 97%, 98%, 99%, or 100%. In some embodiments, the sensitivity can reach any of about 60%, about 65%, about 70%, about 75%, about 80%, about 85%, about 90%, about 95%, about 96%, about 97%, about 98%, about 99%, or about 100%. In some embodiments, the sensitivity can reach about 75%, about 76%, about 77%, about 78%, about 79%, about 80%, about 81%, about 82%, about 83%, about 84%, about 85%, or higher.

In some embodiments, the reference number is about 2 to about 15 SE TCR clones. In some embodiments, the reference number is about any of 2, 3, 4, 5, 6, 7, 8, 9, 10, 11, 12, 13, 14, or 15 SE TCR clones. In some embodiments, the reference number is six SE TCR clones.

In some embodiments, the reference number is about 2 to about 15 de novo SE TCR clones. In some embodiments, the reference number is about any of 2, 3, 4, 5, 6, 7, 8, 9, 10, 11, 12, 13, 14, or 15 de novo SE TCR clones. In some embodiments, the reference number is six de novo SE TCR clones.

In some embodiments, the reference frequency is about 10⁻² to about 10⁻⁶ de novo SE TCR clones. In some embodiments, the reference frequency is about any of 10⁻², 10^{-3, 104}, 10⁻⁵, or 10⁻⁶ de novo SE TCR clones. In some embodiments, the reference frequency is about 10⁻⁴ de novo SE TCR clones.

### Selecting the Patient as More Likely to Respond to the Therapy Comprising the Individualized RNA Vaccine

In some embodiments, a number and/or frequency of de novo SE TCR clones in the sample from the patient above a reference number and/or frequency indicates that the patient is more likely to respond to the therapy comprising the individualized RNA vaccine. The number and/or frequency of de novo SE TCR clones correlates to the immunogenic response or immunogenicity to the individualized RNA vaccine. Increased immunogenic response or immunogenicity to the individualized RNA vaccine is correlated to and indicative of increased treatment responsiveness.

With access to the TCR repertoire (e.g., the number and/or frequency of de novo SE TCR clones) in peripheral blood at baseline (i.e., pre-treatment) and after onset of administering the treatment, a predictive model can detect the immune response to the individualized RNA vaccine based on the number of de novo SE TCR clones. Machine-learning approaches can improve further the prediction algorithm. In some embodiments, the patient is selected as likely to respond to a therapy comprising an individualized RNA vaccine using a predictive model. In some embodiments, the patient is selected as likely to respond to a therapy comprising an individualized RNA vaccine using a computer-based predictive model. In some embodiments, the patient is selected as likely to respond to a therapy comprising an individualized RNA vaccine using machine-learning. In some embodiments, the patient is selected as likely to respond to a therapy comprising an individualized RNA vaccine based on the number and/or frequency of SE TCR clones. In some embodiments, the patient is selected as likely to respond to a therapy comprising an individualized RNA vaccine based on the number and/or frequency of de novo SE TCR clones. In some embodiments, the patient is selected as likely to respond to a therapy comprising an individualized RNA vaccine when the number and/or frequency of de novo SE TCR clones is above the reference number and/or frequency.

In some embodiments, the patient is selected as likely to respond to a therapy comprising an individualized RNA vaccine when the number of SE TCR clones is more than about any of three, four, five, six, seven, eight, nine, ten, or more SE TCR clones. In some embodiments, the patient is selected as likely to respond to a therapy comprising an individualized RNA vaccine when the number of de novo SE TCR clones is more than about any of three, four, five, six, seven, eight, nine, ten, or more de novo SE TCR clones. In some embodiments, the patient is selected as likely to respond to a therapy comprising an individualized RNA vaccine when the number of de novo SE TCR clones is more than six de novo SE TCR clones.

In some embodiments, the patient is selected as likely to respond to a therapy comprising an individualized RNA vaccine when the frequency of de novo SE TCR clones is greater than about any of 10⁻⁶, 10⁻⁵, 10⁻⁴, 10⁻³, or 10⁻² de novo SE TCR clones. In some embodiments, the patient is selected as likely to respond to a therapy comprising an individualized RNA vaccine when the frequency of de novo SE TCR clones is greater than 10⁻⁴ de novo SE TCR clones.

### OTHER EMBODIMENTS

Also provided herein is a method of treating a patient (such as a human patient) having a cancer tumor, wherein the method comprises: a) measuring the number and/or frequency of de novo significantly expanded (SE) TCR clones in a sample from the patient by T cell receptor sequencing; b) comparing the number and/or frequency of de novo SE TCR clones measured in a) to a reference number and/or frequency; and c) selecting the patient as more likely to respond to the therapy comprising the individualized RNA vaccine when the number and/or frequency of de novo SE TCR clones in the sample from the patient is above the reference number and/or frequency; wherein the individualized RNA vaccine comprises one or more polynucleotides encoding one or more neoepitopes resulting from cancer-specific somatic mutations present in a cancer tumor specimen obtained from the patient, and wherein a number and/or frequency of de novo SE TCR clones above the reference number and/or frequency indicates that the patient is more likely to respond to the therapy comprising the individualized RNA vaccine.

In another aspect is provided a method of treating a human patient having a cancer tumor, the method comprising: a) comparing the number and/or frequency of de novo significantly expanded (SE) TCR clones in a sample from the patient to a reference number and/or frequency; and b) selecting the patient as more likely to respond to the therapy comprising the individualized RNA vaccine when the number and/or frequency of de novo SE TCR clones in the sample from the patient is above the reference number and/or frequency, thereby treating the cancer tumor; wherein the individualized RNA vaccine comprises one or more polynucleotides encoding one or more neoepitopes resulting from cancer-specific somatic mutations present in a cancer tumor specimen obtained from the patient, wherein the number and/or frequency of de novo SE TCR clones is measured by T cell receptor sequencing, and wherein a number and/or frequency of de novo SE TCR clones above the reference number and/or frequency indicates that the patient is more likely to respond to the therapy comprising the individualized RNA vaccine.

In some embodiments, the method further comprises administering the therapy comprising the individualized RNA vaccine to the patient when the number and/or frequency of de novo SE TCR clones in the sample from the patient is above the reference number and/or frequency, thereby treating the cancer tumor. In some embodiments, the method further comprises selecting the therapy comprising the individualized RNA vaccine when the number and/or frequency of de novo SE TCR clones in the sample from the patient is above the reference number and/or frequency, thereby treating the cancer tumor.

In some embodiments, the cancer tumor is a pancreatic cancer tumor. In some embodiments, the cancer tumor is a pancreatic ductal adenocarcinoma (PDAC) tumor.

In some embodiments, the therapy comprising the individualized RNA vaccine further comprises a PD-1 axis binding antagonist. In some embodiments, the PD-1 axis binding antagonist is atezolizumab.

In some embodiments, the therapy further comprises a chemotherapeutic treatment and wherein the RNA vaccine, the PD-1 axis binding antagonist, and the chemotherapeutic treatment are administered to the patient during a priming phase, a chemotherapy phase after the priming phase, and a boost phase after the chemotherapy phase, wherein: (i) the priming phase comprises administering to the patient at least one dose of the RNA vaccine and at least one dose of the PD-1 axis binding antagonist, (ii) the chemotherapy phase comprises administering to the patient the chemotherapeutic treatment, and (iii) the boost phase comprises administering to the patient at least one dose of the RNA vaccine and at least one dose of the PD-1 axis binding antagonist. In some embodiments, the chemotherapeutic treatment is a FOLFIRINOX treatment or an mFOLFIRINOX treatment.

In some embodiments of the methods of treatment described in the above sections, prior to the administering step, the patient is selected by the patient selection methods described above.

In some embodiments, the steps of the methods are performed by a single entity. In other embodiments, individual steps may be performed by different entities. For example, the step of measuring the number and/or frequency of de novo SE TCR clones, may be performed by an entity distinct from that which performs the comparing and selecting steps described above.

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

### EXAMPLES

The present disclosure will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: a Phase II, Open-Label, Multicenter, Randomized Study of the Efficacy and Safety of the Individualized Cancer Vaccine Plus Atezolizumab and mFOLFIRINOX Versus mFOLFIRINOX Alone in Patients with Resected Pancreatic Ductal Adenocarcinoma

This Example describes a Phase II, open-label, multicenter, randomized study evaluating the efficacy and safety of the individualized cancer vaccine plus atezolizumab and modified leucovorin, 5 fluorouracil (5-FU), irinotecan, and oxaliplatin (mFOLFIRINOX) versus mFOLFIRINOX in patients with resected PDAC who have not received prior systemic anti-cancer treatment for PDAC and have no evidence of disease after surgery. This study aims to identify a more effective adjuvant therapy for PDAC, because the majority of patients who undergo PDAC resection followed by current standard-of-care adjuvant therapy with gemcitabine combination therapy or mFOLFIRINOX experience disease recurrence and death.

### Study Objectives

The objectives of this study are to evaluate the efficacy and safety of the individualized cancer vaccine plus atezolizumab and mFOLFIRINOX versus mFOLFIRINOX in patients with resected PDAC.

### Study Design

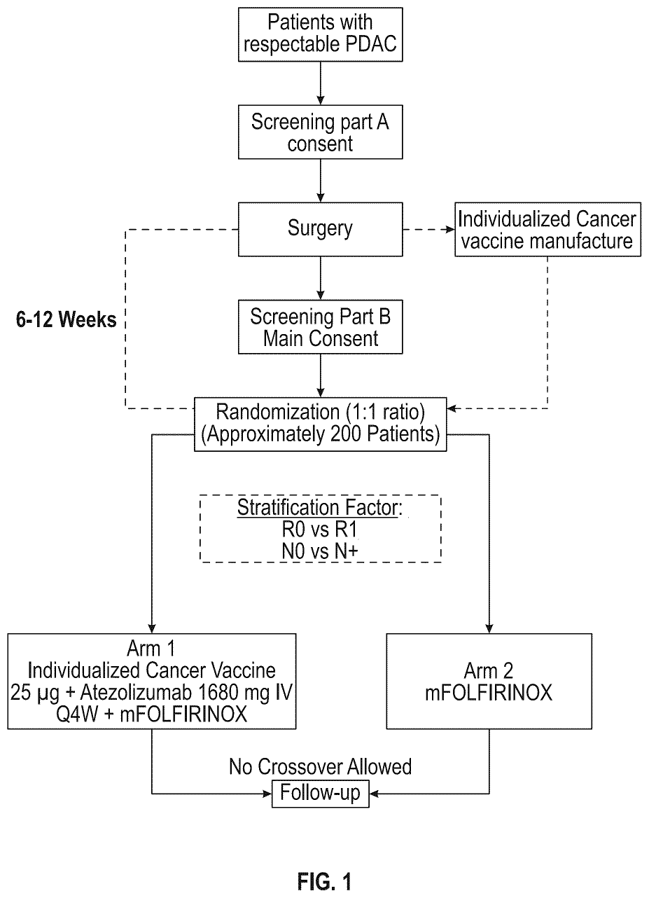

This study enrolls approximately 260 patients at approximately 80 sites globally. As shown in FIG. 1, the Phase II study includes i) a two-part screening period (Part A and Part B); ii) a treatment period consisting of one or three phases (priming, chemotherapy, and boost), depending on the treatment arm; and iii) a follow-up period. The total duration of study participation for each patient is expected to range from 1 day to more than 6 years.

Screening occurs in two parts, termed Part A and Part B. During Part A, blood and tumor tissue specimens are tested to determine the presence of at least five neoepitopes to enable manufacturing of each patient's individualized cancer vaccine. Patients further undergo limited screening for eligibility and a review of medical history. During screening Part B, patient eligibility is confirmed, including assessments collected within 28 and 14 days of enrollment. Patients are randomized to Arm 1 or Arm 2; a stratified, permuted-block randomization scheme is used to obtain an approximately 1:1 ratio between the two treatment arms. Randomization is stratified by resection margin status (R0 vs. R1) and lymph node involvement (N0 vs. N+). Study treatment then begins within 7 days of randomization.

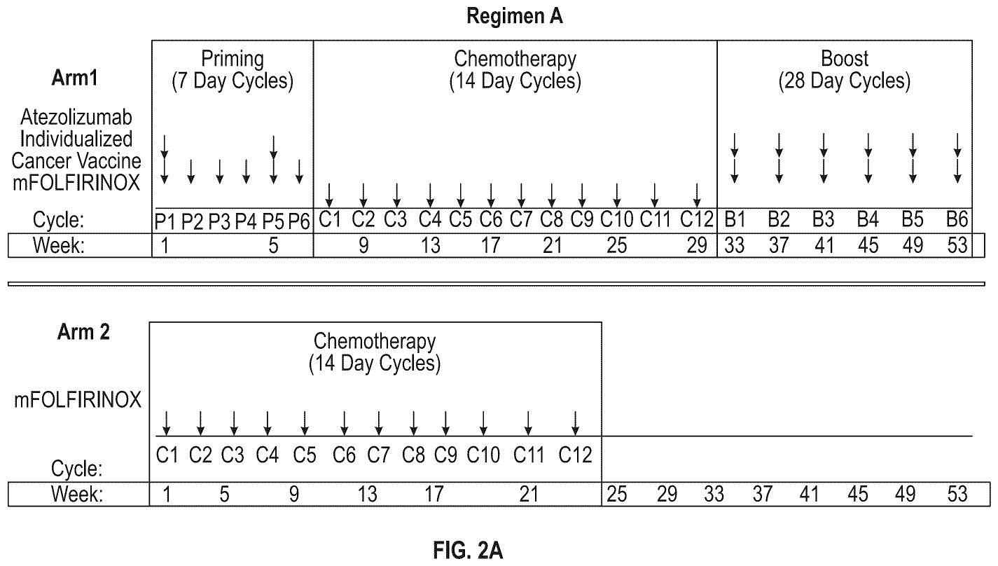

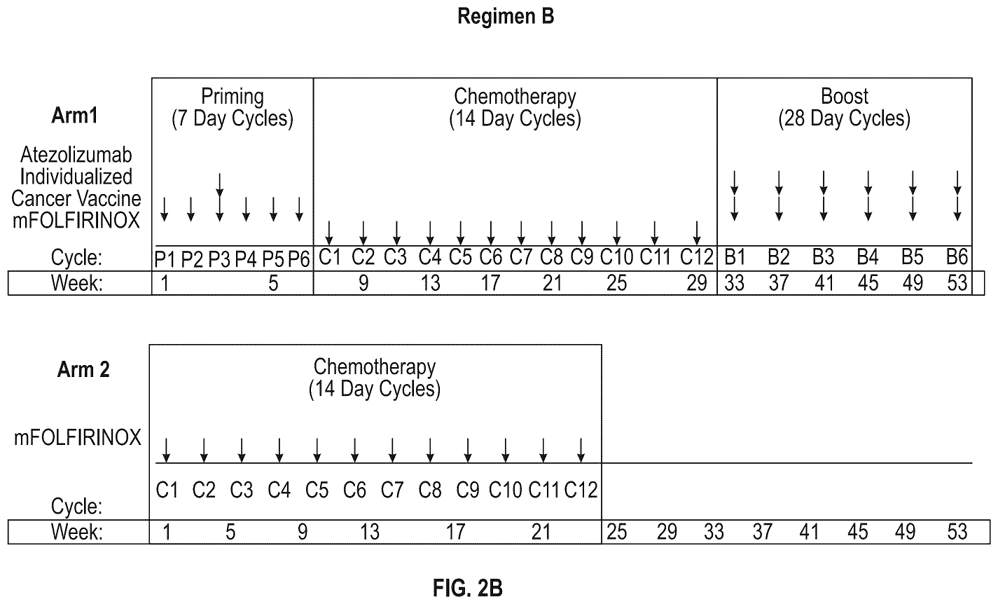

Two alternative dosing regimens are used in the study, dosing regimen A and dosing regimen B, as shown in FIGS. 2A-2B.

An overview of the dosing regimen A for arms 1 and 2 is shown in FIG. 2A. Patients in the experimental arm (Arm 1) receive the individualized cancer vaccine, atezolizumab, and mFOLFIRINOX, given over three phases of treatment, with approximate timing as follows:

### Priming Phase (Weeks 1-6)·

Individualized cancer vaccine 25 μg IV in 7-day cycles starting on Day 1 of Week 1, for a total of six cycles (six doses).
Atezolizumab 1680 mg IV on Day 1 of Week 1 and Day 1 of Week 5, for a total of two doses.

### Chemotherapy Phase (Weeks 7-29)·

mFOLFIRINOX (oxaliplatin 85 mg/m², leucovorin 400 mg/m², irinotecan 150 mg/m², 5-FU 2400 mg/m²) IV in 14-day cycles starting on Day 1 of Week 7, for a total of up to 12 cycles (12 administrations).

### Boost Phase (Weeks 33-53)·

Individualized cancer vaccine 25 μg IV in 28-day cycles starting on Day 1 of Week 33, for a total of six cycles (six doses).
Atezolizumab 1680 mg IV in 28-day cycles starting on Day 1 of Week 33, for a total of six cycles (six doses).

An overview of the dosing regimen B for arms 1 and 2 is shown in FIG. 2B. The Chemotherapy and Boost Phases of regimen B are the same as the corresponding phases in regimen A. The Priming Phase in regimen B is modified as follows:

### Priming Phase (Weeks 1-6)·

Individualized cancer vaccine 25 μg IV in 7-day cycles (i.e., once per week, QW) starting on Day 1 of Week 1, for a total of six doses.
Atezolizumab 1680 mg IV administered on Day 1 of Week 3 for a total of one dose.

The Priming Phase of regimen B was designed to increase immunogenicity and clinical activity of the individualized cancer vaccine when administered in combination with atezolizumab. Biomarker samples are collected from patients who have received autogene cevumeran but have not yet received atezolizumab.

PDAC is considered a “cold” tumor (Karamitopoulou. Br J Cancer. 2019; 121, 5-14), also referred to as an immunological desert, characterized by a lack of PD-L1 expression and non-responsiveness to immune checkpoint inhibitor (CPI) treatment (Li et al. Cell Commun Signal, 2021; 19, 117). PD-L1 expression in cancer cells correlates with responsiveness to immune checkpoint inhibitors (Kwon et al. Journal of Controlled Release. 2021; 331, pp. 321-334). A PD-L1-adaptive upregulation can be induced by cancer vaccine-induced T cells and the release of interferon-γ. Hence, priming the immune system with the individualized cancer vaccine, and the resulting priming of T cells as well as PD-L1 upregulation, may increase the response to checkpoint blockade (e.g., PD1/PD-L1 inhibitors) (Ribas et al. J Exp Med. 2016 Dec. 12; 213 (13): 2835-2840; Ye et al. J Cancer. 2018 Jan. 1; 9 (2): 263-268). The priming and first round of substantial expansion of vaccine-induced T cells typically takes about 2-3 weeks. Checkpoint inhibitors, such as an anti-PD-L1 agent, may synergistically enhance the activity of cancer vaccine-induced T cells within the tumor microenvironment after their development (Collins et al. Expert Rev Vaccines. 2018 August; 17 (8): 697-705). On the other hand, it has been suggested that administering checkpoint inhibitors before a cancer vaccine might negatively impact vaccine response by activating CD38+PD1+immune suppressive T cells (Verma et al. Nat Immunol. 2019; 20, 1231-1243). Accordingly, the Priming Phase of regimen B includes 2 priming doses of the individualized cancer vaccine administered prior to atezolizumab (and prior to initiation of chemotherapy), which may lead to improved immunogenicity and clinical activity.

Patients in the control arm (Arm 2) receive mFOLFIRINOX (oxaliplatin 85 mg/m², leucovorin 400 mg/m², irinotecan 150 mg/m², 5 FU 2400 mg/m²) IV in 14-day cycles starting on Day 1 of Week 1, for a total of up to 12 cycles (12 administrations).

Patients undergo imaging and other cancer-related assessments at screening and scheduled intervals during the study until recurrence of PDAC or occurrence of new cancer. Images for cancer-related assessments are prospectively collected for all patients to enable retrospective, blinded, independent central review as needed. Cancer-related assessments include biochemical testing of carcinoembryonic antigen [CEA] and CA19-9, clinical evaluation for signs and symptoms of disease, and tumor assessments (cancer-related imaging assessments). Blood samples for exploratory biomarker analyses, pharmacokinetic analyses, and immunogenicity analyses are collected at various timepoints.

All patients are closely monitored for adverse events during study treatment and for 90 days after the final dose of study treatment. Adverse events are graded according to the National Cancer Institute's Common Terminology Criteria for Adverse Events (CTCAE), Version 5.0.

Patients who complete study treatment or discontinue study treatment prior to receiving all planned doses for reasons other than recurrence of PDAC or occurrence of new cancer return to the clinic for a treatment discontinuation visit 28 (+7) days after the final dose of study treatment and then enter follow-up. During follow-up, patients continue to undergo cancer-related assessments and other assessments (e.g., collection of blood samples for TCR sequencing) until investigator-assessed recurrence of PDAC or occurrence of new cancer.

The end of this study is defined as the date at which the last data required for all study analyses have been collected. The end of the study is expected to occur approximately 7.5 years after the first patient is randomized.

### Study Participants

### Inclusion Criteria

Patients that meet the following criteria are included in this study:
Eastern Cooperative Oncology Group (ECOG) performance status of 0 or 1
Preoperative diagnosis of resectable PDAC tumor, as demonstrated by preoperative imaging with computed tomography (CT) scan with contrast or magnetic resonance imaging (MRI) scan per institutional standard for imaging evaluation of pancreas (e.g., CT pancreas protocol) and defined as meeting all of the following radiographic criteria:
   Clear fat plane around the celiac and superior mesenteric arteries
   Patent superior mesenteric and portal veins
   No encasement of the superior mesenteric vein or portal veins
   No encasement of the superior mesenteric or hepatic arteries
   Absence of metastatic disease
   Absence of extra-regional nodal disease
Histologically confirmed diagnosis of PDAC
Pancreatic cancer tumor, lymph node, metastasis (TNM) pathological staging values of T1-T3, N0-N2, and MO per the American Joint Committee on Cancer (AJCC) Cancer Staging Manual, 8th edition
   Patients with staging values of Tx, T4, Nx, or Ml are not eligible for the study
Macroscopically complete (R0 or R1) resection of PDAC
Unequivocal absence of disease after surgery as assessed by the investigator and based on review of all available data including mandatory imaging (CT or MRI scans), biochemical data, and clinical findings within 28 days prior to randomization
CA19-9 level measured within 14 days prior to randomization
Presence of at least five tumor neoepitopes as identified from blood and tumor tissue submitted during screening Part A.
Interval of between 6 and 12 weeks since resection of PDAC
Full recovery from surgery and ability to receive atezolizumab, the individualized cancer vaccine, and mFOLFIRINOX in the investigator's judgment
Adequate hematologic and end-organ function, defined by laboratory test results, obtained within 14 days prior to initiation of study treatment
For patients receiving therapeutic anticoagulation: stable anticoagulant regimen
Negative HIV test at screening
No evidence of active hepatitis B, defined as having a positive hepatitis B surface antigen (HBsAg) test) at screening
Negative hepatitis C virus (HCV) antibody test at screening, or positive HCV antibody test followed by a negative HCV RNA test at screening.

### Exclusion Criteria

Patients that meet the following criteria are excluded from this study:
Prior adjuvant, neoadjuvant, or induction treatment for pancreatic cancer, including cytotoxic chemotherapy, immunotherapy, investigational therapy, or radiation therapy
Absence of spleen (due to splenectomy, splenic injury/infarction, or functional asplenia)
Preexisting neuropathy
Known presence of aUGT1A1 genotype associated with poor metabolizer phenotype
Unresolved≥Grade 3 postoperative complication(s) per the Clavien Dindo Classification of Surgical Complications
Inflammatory disease of the colon or rectum, occlusion or subocclusion of the intestine, or severe postoperative uncontrolled diarrhea
Any serious medical condition or abnormality in clinical laboratory tests that, in the investigator's judgment, precludes the patient's safe participation in and completion of the study
Major surgical procedure, other than for diagnosis or for resection of disease under current study, within <6 weeks prior to initiation of study treatment, or anticipation of need for a major surgical procedure during the study
Significant cardiovascular disease (such as New York Heart Association Class II or greater cardiac disease, myocardial infarction, or cerebrovascular accident) within 3 months prior to initiation of study treatment, unstable arrhythmia, or unstable angina
Clinically significant liver disease, including active viral, alcoholic, or other hepatitis, cirrhosis, and inherited liver disease, or current alcohol abuse as determined by the investigator
Active or history of autoimmune disease or immune deficiency, including, but not limited to, myasthenia gravis, myositis, autoimmune hepatitis, systemic lupus erythematosus, rheumatoid arthritis, inflammatory bowel disease, anti-phospholipid antibody syndrome, Wegener granulomatosis, Sjögren syndrome, Guillain-Barré syndrome, or multiple sclerosis
Known primary immunodeficiencies, either cellular (e.g., DiGeorge syndrome, T negative severe combined immunodeficiency [SCID]) or combined T- and B-cell immunodeficiencies (e.g., T- and B-negative SCID, Wiskott-Aldrich syndrome, ataxia telangiectasia, common variable immunodeficiency)·
History of malignancy within 5 years prior to screening, with the exception of the cancer under investigation in this study and malignancies with a negligible risk of metastasis or death (e.g., 5-year OS rate >90%), such as adequately treated carcinoma in situ of the cervix, non-melanoma skin cancer, localized prostate cancer, ductal carcinoma in situ, or Stage I uterine cancer
Treatment with monoamine oxidase inhibitors (MAOIs) within 3 weeks prior to initiation of study treatment or requirement for ongoing treatment with MAOIs
Treatment with systemic immunostimulatory agents (including, but not limited to, interferon and IL-2) within 4 weeks or 5 drug-elimination half-lives (whichever is longer) prior to initiation of study treatment
Treatment with systemic immunosuppressive medication (including, but not limited to, corticosteroids, cyclophosphamide, azathioprine, methotrexate, thalidomide, and anti-TNF agents) within 2 weeks prior to initiation of study treatment, or anticipation of need for systemic immunosuppressive medication during study treatment
History of idiopathic pulmonary fibrosis, organizing pneumonia (e.g., bronchiolitis obliterans), drug-induced pneumonitis, or idiopathic pneumonitis, or evidence of active pneumonitis on screening chest CT scan
Known active or latent tuberculosis
Recent acute infection, defined as severe infection within 4 weeks prior to initiation of study treatment, including, but not limited to, hospitalization for complications of infection, bacteremia, or severe pneumonia, or any active infection that could impact patient safety
Prior allogeneic stem cell or solid organ transplantation
Any other disease, metabolic dysfunction, physical examination finding, or clinical laboratory finding that contraindicates the use of an investigational drug, may affect the interpretation of the results, or may render the patient at high risk from treatment complications
Treatment with a live, attenuated vaccine within 4 weeks prior to initiation of study treatment, or anticipation of need for such a vaccine during atezolizumab treatment or within 5 months after the final dose of atezolizumab
Receipt of any mRNA vaccine (e.g., COVID-19 vaccine) within 7 days prior to start of study treatment
Current treatment with anti-viral therapy for HBV
History of severe allergic anaphylactic reactions to chimeric or humanized antibodies or fusion proteins
Known hypersensitivity to Chinese hamster ovary cell products or any component of the atezolizumab formulation
Known hypersensitivity or allergy to any component of the individualized cancer vaccine or mFOLFIRINOX formulations, including known hereditary fructose intolerance.

### Study Outcome Measures

The primary outcome measures of this study include the following:
DFS (disease-free survival) after randomization, defined as the time from randomization to one of the following:
   first recurrence of PDAC or first occurrence of new cancer, as determined by the investigator, or
   death from any cause (whichever occurs first).

The secondary outcome measures of this study include the following:
DFS rates at 12, 24, and 36 months, defined as the probability that the patient will not experience recurrence of PDAC or occurrence of new cancer, as determined by the investigator, or death from any cause, at 12, 24, and 36 months after randomization.
OS (overall survival) after randomization, defined as the time from randomization to death from any cause
OS rates at 3 and 5 years, defined as the probability that the patient will be alive at 3 and 5 years after randomization.
Incidence and severity of adverse events, with severity determined according to CTCAE (National Cancer Institute's Common Terminology Criteria for Adverse Events) v5.0 grading scale.
Change from baseline in targeted vital signs.
Change from baseline in targeted clinical laboratory test results.

The exploratory outcome measures of this study include the following:
Change from baseline in physical function, role function, GHS/QOL (global health status/quality of life), and disease-related symptoms at 12 and 18 months as measured through use of the EORTC QLQ-C30 and EORTC PAN-26 (European Organisation for Research and Treatment of Cancer).
Change from baseline in pain as measured through use of the EORTC QLQ-C30 and EORTC PAN-26.
Presence, frequency of occurrence, severity, and/or degree of interference with daily function of symptomatic treatment toxicities, as assessed through use of the PRO-CTCAE (Patient-Reported Outcomes Common Terminology Criteria for Adverse Events).
Change from baseline in symptomatic treatment toxicities, as assessed through use of the PRO-CTCAE.
Degree of bother due to treatment side effects, as assessed through use of the single-item EORTC IL46 (item library 46).
Changes from baseline in antigen-specific T-cell responses.
Longitudinal changes from baseline in biomarkers in blood and tumor tissue.
Relationship between biomarkers in blood and tumor tissue and efficacy, safety, or other biomarker endpoints.
Change from baseline in EuroQol EQ-5D-5L index-based and VAS scores at 12 and 18 months.
Plasma concentrations of DOTMA ((R)-N,N,N-trimethyl-2,3-dioleyloxy-1-propanaminium chloride) and serum concentrations of atezolizumab at specified timepoints.
Prevalence of anti-drug antibodies (ADAs) to atezolizumab at baseline and incidence of ADAs to atezolizumab.
Relationship between plasma concentration or pharmacokinetic (PK) parameters for DOTMA and efficacy endpoints.
Relationship between plasma concentration or PK parameters for DOTMA and safety endpoints.

### Example 2: a Preclinical Mouse Model to Test the Efficacy of the RNA Vaccine in Combination with Modified Mouse FOLFIRINOX

This Example describes a preclinical tumor mouse model evaluating the efficacy of the RNA vaccine plus leucovorin, 5 fluorouracil (5-FU), irinotecan, and oxaliplatin (FOLFIRINOX) compared to FOLFIRINOX alone or no treatment. This study aims to identify effective dosing schedules.

### Study Design

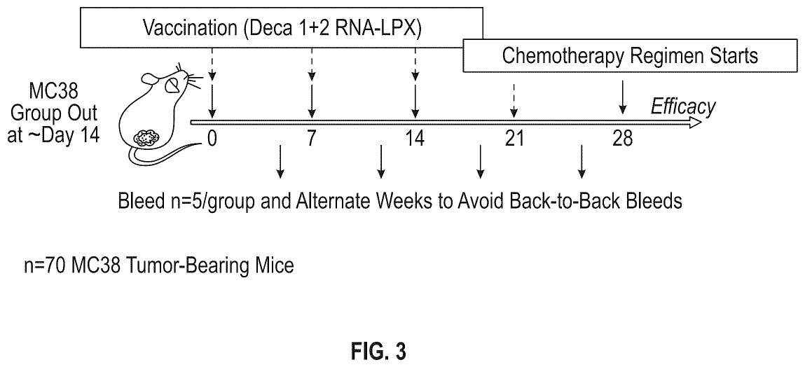

The efficacy of the RNA vaccine in combination with a modified mouse FOLFIRINOX chemotherapy regimen was analyzed in a syngeneic tumor mouse model. FIG. 3 provides a schematic overview of a representative experimental design. The murine colon adenocarcinoma cell line, MC38 was subcutaneously implanted (s.c.) into the right flank of n=140 female C57Bl/6 mice that were approximately 9-10 weeks old. MC38 cells were injected at a concentration of 0.1×10⁶ cells in a volume of 100 μL Hanks' balanced salt solution containing Matrigel at a ratio of 1:1. Tumors were monitored until they reached a tumor volume of 130-250 mm³. Mice with similar tumor volumes were distributed into 8 groups with n=10 mice per group. The mean tumor volume across all 8 groups was 172 mm³ at the initiation of dosing. Tumor sizes and mouse body weights were recorded twice weekly during the study. Mice were promptly euthanized when tumor volume exceeded 2000 mm³ or if body weight loss was >20% of their starting weight.

Each group underwent a different dosing regimen to test the efficacy of each combination RNA-LPX and chemotherapy regimen, compared to the standard of care FOLFIRINOX treatment. The groups were broken out into the following 8 conditions:
Group 1: No treatment
Group 2: Decatope 1+2 RNA-LPX vaccine at days 0, 7, and 14 (QWx3, or once a week for three rounds)
Group 3: Decatope 1+2 RNA-LPX vaccine at days 0, 14, and 28 (Q14Dx3, or once in 14 days for three rounds)
Group 4: FOLFIRINOX (modified mouse dosing, starting at day 0) at days 0, 7, and 14
Group 5: Decatope 1+2 RNA-LPX vaccine at days 0, 7, and 14 (QWx3) and mmFOLFIRINOX at days 0, 7, and 14 (QWx3)
Group 6: Decatope 1+2 RNA-LPX vaccine at days 0, 14, and 28 (Q14Dx3) and mmFOLFIRINOX at days 7, 21, and 35 (Q14Dx3, starting at day 7)
Group 7: Decatope 1+2 RNA-LPX vaccine at days 0, 7, and 14 (QWx3) and mmFOLFIRINOX at days 21, 28, and 35 (QWx3, starting at day 21)
Group 8: Decatope 1+2 RNA-LPX vaccine at days 0, 7, and 14 (QWx3) and mmFOLFIRINOX at days 14, 21, and 28 (QWx3, starting at day 14)

Modified mouse FOLFIRINOX was administered at the following dose concentrations: Oxaliplatin at 5 mg/kg via i.p. injection; Irinotecan at 20 mg/kg via i.p. injection; Leucovorin at 100 mg/kg via i.p. injection; and Fluouracil at 25 mg/kg via i.p. injection plus 25 mg/kg via subcutaneous injection (s.c.). Leucovorin dosing immediately followed oxaliplatin dosing, and irinotecan immediately followed leucovorin dosing. Fluorouracil was administered 2 hours after other chemotherapy dosing was completed.

The RNA vaccine was formulated as an RNA-LPX (lipoplex) and was administered in 3 doses at 50 μg per mouse in a volume of 200 μL by intravenous injection (i.v.), starting on day 0.

Table 5 below provides an overview of the treatment schedule for each group.

TABLE 5
Treatment schedules for each group in the MC38 murine tumor study.

| | | | | | | | Chemo |
|---|---|---|---|---|---|---|---|
| | | RNA- | | | Vaccination | Vaccine | regimen |
| Group | Animal # | LPX | Dose | Chemo | Schedule | Start | Start |
| Group-1 | 10 | — | — | — | — | — | |
| Group-2 | 10 | Decal + 2 | 50 ug | — | q7dx3 | Day 0 | — |
| Group-3 | 10 | Decal + 2 | 50 ug | — | q14dx3 | Day 0, | — |
| | | | | | | q14dx3 | |
| Group-4 | 10 | — | — | FOLFIRINOX | — | — | Day 0 |
| Group-5 | 10 | Decal + 2 | 50 ug | FOLFIRINOX | q7dx3 | Day 0 | Day 0 |
| Group-6 | 10 | Decal + 2 | 50 ug | FOLFIRINOX | q14dx3 | Day 0 | Day 7, |
| | | | | | | | q14dx3 |
| Group-7 | 10 | Decal + 2 | 50 ug | FOLFIRINOX | q7dx3 | Day 0 | Day 21 |
| Group-8 | 10 | Decal + 2 | 50 ug | FOLFIRINOX | q7dx3 | Day 0 | Day 14 |

### Results

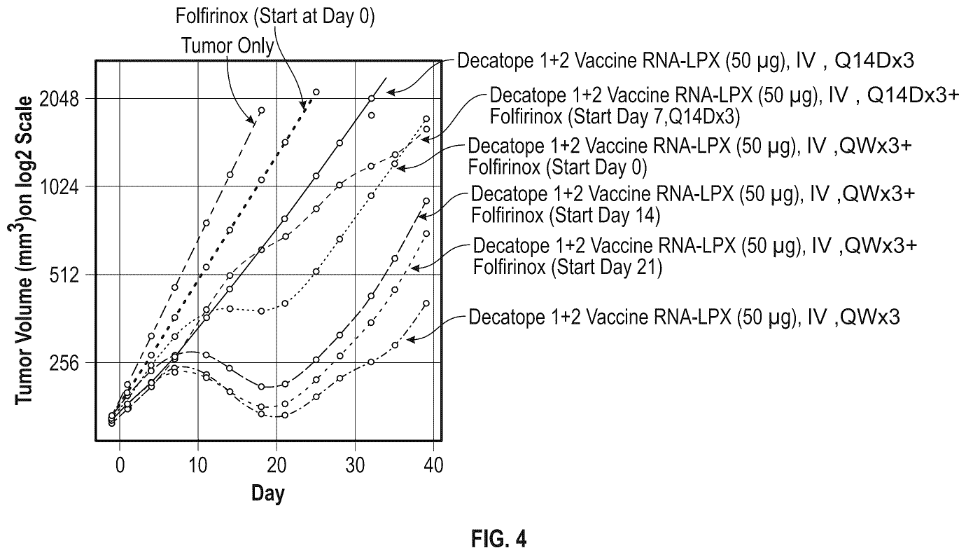

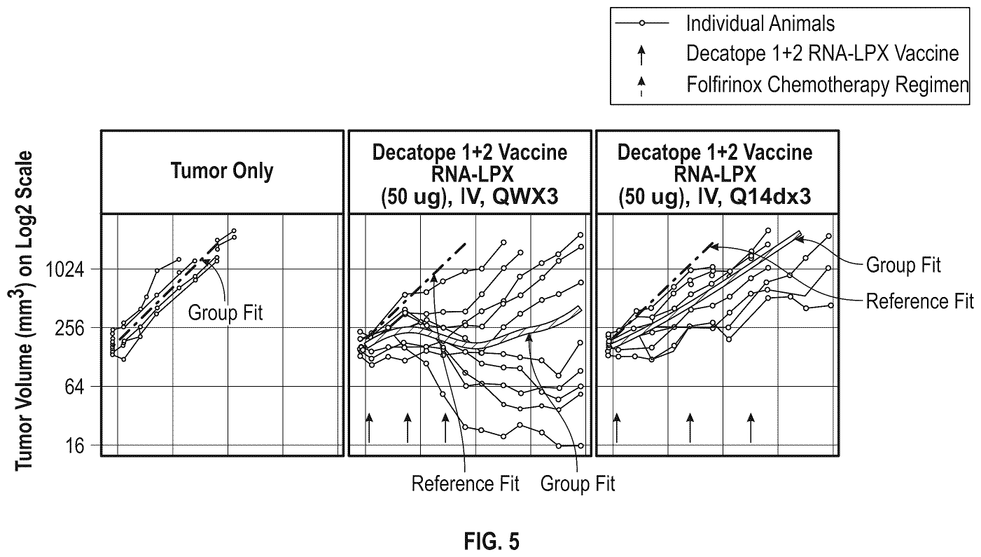

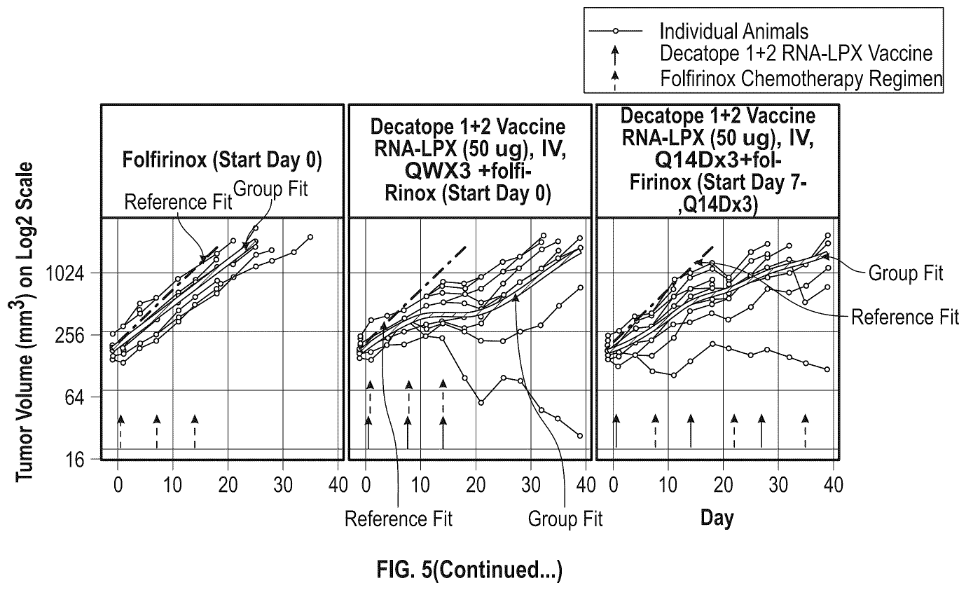

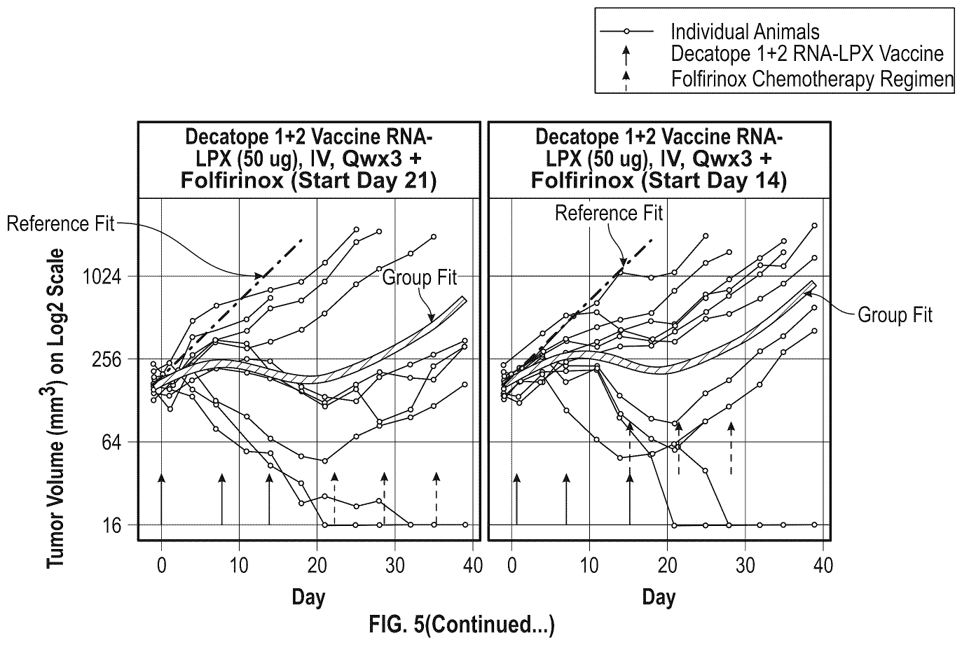

Average results for each group can be found in Table 6 below. FIG. 4 shows the best-fit tumor growth curve for each group (i.e., Groups 1-8). FIG. 5 shows the tumor growth curves for each individual mouse within a single group with a group best fit curve and a reference fit overlaid for each group (i.e., Groups 1-8).

As shown in FIG. 4, mice that were vaccinated with RNA-LPX without receiving chemotherapy showed significant tumor growth delay relative to untreated control (Group 2 versus Group 1, TGI 91). Concurrent administration of RNA-LPX with the modified murine FOLFIRINOX regimen significantly abrogated this tumor growth control (Group 2, TGI 91 versus Group 5, TGI 71). Alternating weekly RNA-LPX with weekly mmFOLFIRNOX also resulted in poor tumor growth control (Groups 3 and 6). Delay of chemotherapy initiation until either 2 (Group 8) or 3 (Group 7) doses of RNA-LPX were administered restored anti-tumor activity (Group 2, TGI 90 versus Group 8 and Group 7, TGI 87 and 91 respectively).

TABLE 6
Results of tumor growth assessment for RNA-LPX and/or mmFOLFIRINOX administration.

| | | | | | | | Legacy % | | | |
|---|---|---|---|---|---|---|---|---|---|---|
| | | | | | | N | TGI | | | |
| | | N | Vaccine | Chemo | Last | Last | (Lower, | | EOS | TTP |
| Number | Group Name | total | Start | Start | Day | Day | Upper) | PR | CR | 2x |
| 1 | tumor only | 10 | — | — | 21 | 2 | 0 | 0 | 0 | 4.5 |
| | | | | | | | (0, 0) | (0.0%) | (0.0%) | |
| 2 | Decatope 1 + 2 | 10 | Day 0 | — | 39 | 8 | 91 | 5 | 0 | 36.2 |
| | vaccine RNA- | | | | | | (75, 100) | (50.0%) | (0.0%) | |
| | LPX (50 ug), | | | | | | | | | |
| | IV, QWx3 | | | | | | | | | |
| 3 | Decatope 1 + 2 | 10 | Day 0 | — | 39 | 4 | 68 | 0 | 0 | 9.3 |
| | vaccine RNA- | | | | | | (36, 87) | (0.0%) | (0.0%) | |
| | LPX (50 ug), | | | | | | | | | |
| | IV, Q14Dx3 | | | | | | | | | |
| 4 | Folfirinox (start | 10 | — | Day 0 | 35 | 1 | 40 | 0 | 0 | 6.1 |
| | day 0) | | | | | | (−20, 71) | (0.0%) | (0.0%) | |
| 5 | Decatope 1 + 2 | 10 | Day 0 | Day 0 | 39 | 4 | 71 | 1 | 0 | 7.3 |
| | vaccine RNA- | | | | | | (43, 87) | (10.0%) | (0.0%) | |
| | LPX (50 ug), | | | | | | | | | |
| | IV, QWx3 + | | | | | | | | | |
| | Folfirinox (start | | | | | | | | | |
| | day 0) | | | | | | | | | |
| 6 | Decatope 1 + 2 | 10 | Day 0 | Day 7 | 39 | 5 | 66 | 1 | 0 | 9.1 |
| | vaccine RNA- | | | | | | (29, 85) | (10.0%) | (0.0%) | |
| | LPX (50 ug), | | | | | | | | | |
| | IV, Q14Dx3 + | | | | | | | | | |
| | Folfirinox (start | | | | | | | | | |
| | day 7, Q14Dx3) | | | | | | | | | |
| 7 | Decatope 1 + 2 | 10 | Day 0 | Day 21 | 39 | 6 | 91 | 4 | 0 | 31.0 |
| | vaccine RNA- | | | | | | (76, 101) | (40.0%) | (0.0%) | |
| | LPX (50 ug), | | | | | | | | | |
| | IV, QWx3 + | | | | | | | | | |
| | Folfirinox (start | | | | | | | | | |
| | day 21) | | | | | | | | | |
| 8 | Decatope 1 + 2 | 10 | Day 0 | Day 14 | 39 | 6 | 87 | 3 | 0 | 28.9 |
| | vaccine RNA- | | | | | | (69, 98) | (30.0%) | (0.0%) | |
| | LPX (50 ug), | | | | | | | | | |
| | IV, QWx3 + | | | | | | | | | |
| | Folfirinox (start | | | | | | | | | |
| | day 14) | | | | | | | | | |

These results support the selection of a dosing regimen with at least 2 or 3 priming doses of RNA vaccine before initiation of chemotherapy.

### Example 3: Results from a Phase Ia/Ib Study of an Individualized RNA Vaccine as a Single Agent and in Combination with Atezolizumab in Patients with Locally Advanced or Metastatic Tumors

This Example describes results from a Phase Ia/Ib, open-label, multicenter, global, dose-escalation study designed to evaluate the safety, tolerability, immune response, and pharmacokinetics of an individualized RNA vaccine as a single agent and in combination with the anti-PD-L1 antibody atezolizumab. The Phase Ia/Ib study was carried out as described in Examples 1-5 of PCT/US2021/015710, which is hereby incorporated by reference in its entirety.

### Study Design

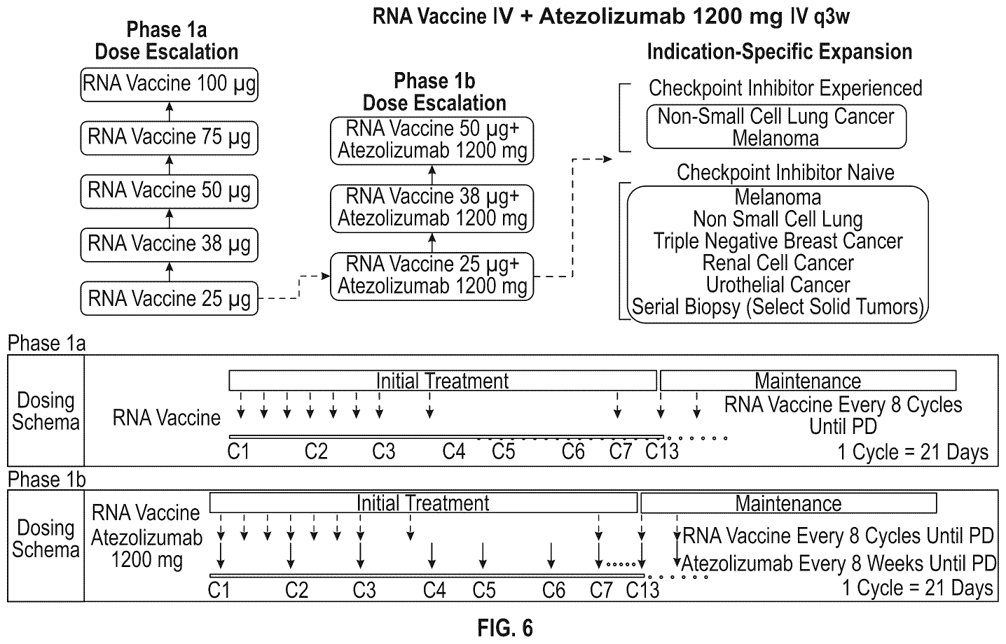

As shown in FIG. 6, patients in the Phase Ia dose escalation study were administered the individualized RNA vaccine as a monotherapy at doses of 25 μg, 38 μg, 50 μg, 75 μg, or 100 μg. During initial treatment (induction stage), the RNA vaccine was administered on Days 1, 8, and 15 of Cycle 1, on Days 1, 8, and 15 of Cycle 2, on Days 1 and 15 of Cycle 3, and on Day 1 of Cycle 7 (each cycle was 21 days). During the maintenance stage after initial treatment, the RNA vaccine was administered on Day 1 of Cycle 13, and every 8 cycles thereafter (i.e., every 24 weeks thereafter, or every 168 days thereafter) until disease progression (PD) (each cycle was 21 days).

Patients in the Phase Ib study were administered the RNA vaccine in doses of 15 μg (not shown), 25 μg, 38 μg, or 50 μg in combination with 1200 mg atezolizumab. The Phase Ib study included a dose escalation phase for the RNA vaccine and an expansion phase in which patients with the indicated checkpoint inhibitor naïve or checkpoint inhibitor experienced tumor types were administered the RNA vaccine at a dose of 15 μg or 25 μg in combination with atezolizumab (additional tumor types in the Phase Ib expansion phase are provided in Example 1 of PCT/US2021/015710). During initial treatment (induction stage), atezolizumab was administered on Day 1 of each of Cycles 1-12; and the RNA vaccine was administered on Days 1, 8, and 15 of Cycle 1, on Days 1, 8, and 15 of Cycle 2, on Days 1 and 15 of Cycle 3, and on Day 1 of Cycle 7 (each cycle was 21 days). During the maintenance stage after initial treatment, atezolizumab was administered every 3 weeks until disease progression (PD), starting on Day 1 of Cycle 13; and the RNA vaccine was administered on Day 1 of Cycle 13, and every 8 cycles thereafter (i.e., every 24 weeks thereafter, or every 168 days thereafter) until disease progression (PD) (each cycle was 21 days).

pMHC Multimer Assay

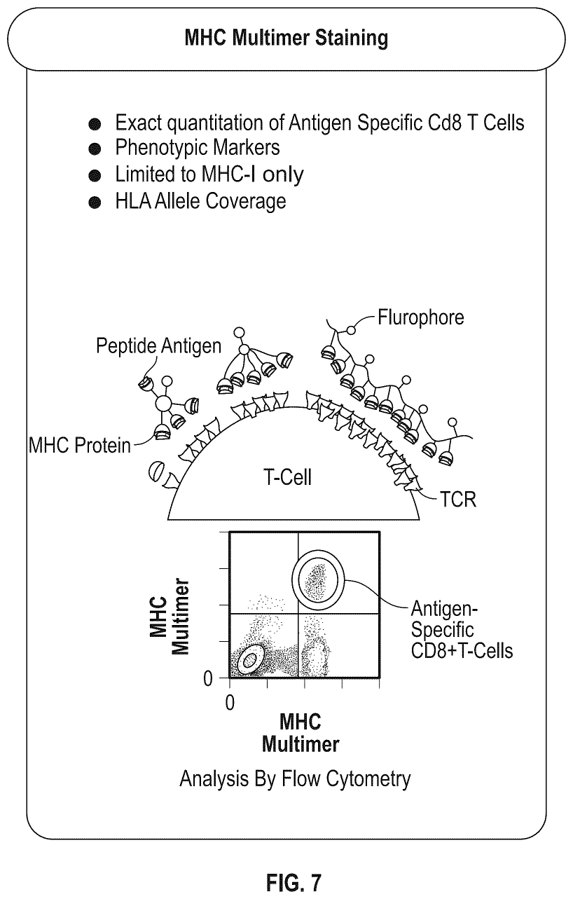

Individual pMHC multimers were designed for each patient based on the patient's HLA class I allele and using peptides derived from predicted epitopes in the neoantigen targets used in the RNA vaccine. Frozen peripheral blood mononuclear cells (PBMCs) were used for fluorescence activated cell sorting (FACS) staining. Each sample was stained with multiple pMHC multimers and additional antibodies for defining the phenotypes of neoantigen-specific CD8+ T cells. FACS panels were designed such that each neoantigen had two pMHC multimers labeled with two different fluorophores (to increase the specificity of the staining). CD8+ T cells were gated among the PBMCs and analyzed for staining with the two pMHC multimers labeled with two different fluorophores for each neoantigen. In order for any given CD8+ T cell to be called positively stained (i.e., neoantigen-specific), it had to stain positive for both of the pMHC multimers labeled with two different fluorophores and fall in the top right quadrant in the FACS plot. A diagram of the pMHC multimer staining assay methods is provided in FIG. 7.

### Results

### Six Priming RNA Vaccine Doses Result in Higher CD8+ T Cell Responses

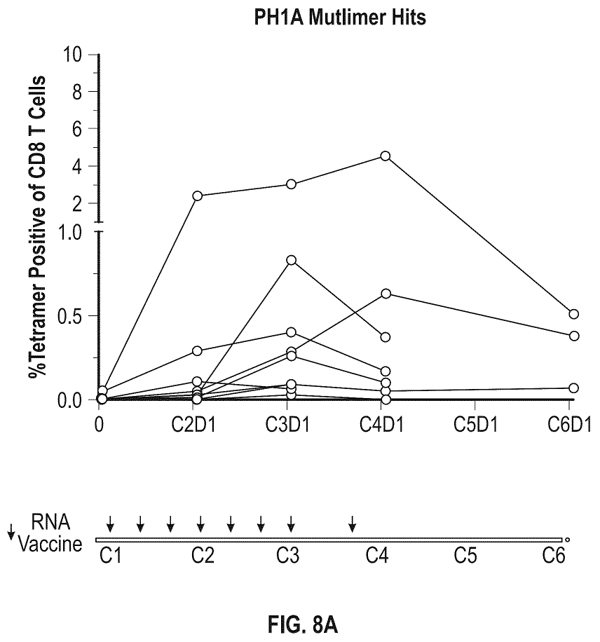

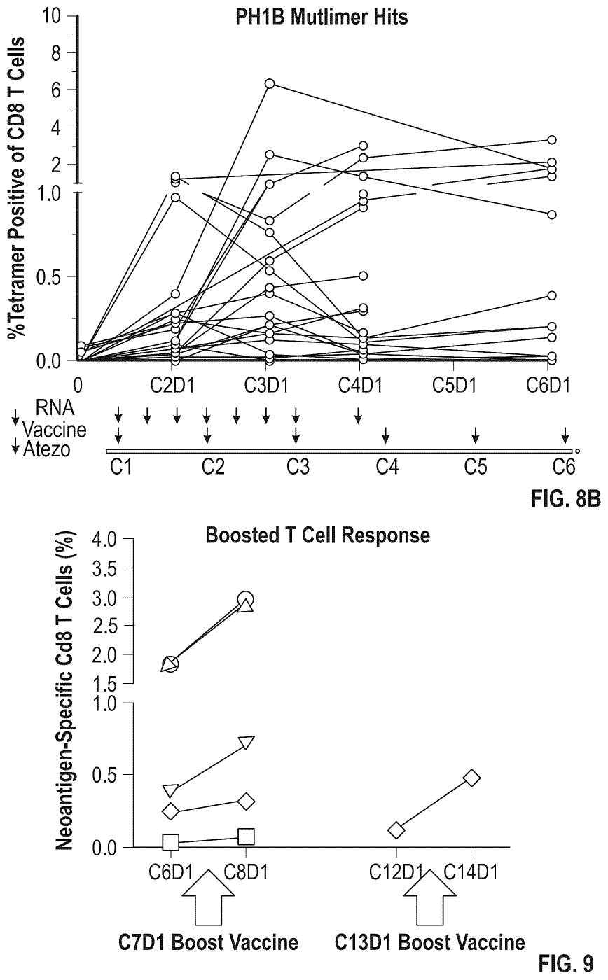

Cancer types in patients in the Phase Ia and Phase Ib studies include triple negative breast cancer, ovarian cancer, colon cancer, prostate cancer, and cervical cancer. As shown in FIGS. 8A-8B, in patients in the Phase Ia and Phase Ib studies, the level of CD8+ T cell responses in peripheral blood, measured by pMHC staining, reached peak of immune responses at Day 1 of Cycle 3, i.e., after 6 doses of the individualized RNA vaccine. The median frequency of vaccine target-specific CD8+ T cells in the Phase Ia individualized RNA vaccine monotherapy cohort was 0.02% at Day 1 of Cycle 2 and increased roughly 9-fold to 0.175% at Day 1 of Cycle 3 (n=11 hits) (FIG. 8A). Similarly, in the Phase Ib cohort (individualized RNA vaccine in combination with atezolizumab) the median of immune response increased by 3-fold from 0.12% at Day 1 of Cycle 2 to 0.34% at Day 1 of Cycle 3 (n=23 hits) (FIG. 8B). Of the CD8+ T cell responses that were followed further at Day 1 of Cycle 4 and beyond up to Day 1 of Cycle 6, the levels of antigen-specific CD8+ T cells did not increase further during this period. These data strongly support the administration of at least 6 vaccine doses during a priming phase of treatment to stimulate optimal CD8+ T cell responses.

### Additional Boost Doses of RNA Vaccine Further Augment CD8+ T Cell Responses in Blood

Immune monitoring data was available from 5 patients who received an individualized RNA vaccine boost dose (from both the Phase Ia study and the Phase Ib study). The patient cancer types are described in Table 7. Further tracking of CD8 T cell responses at Day 1 of Cycle 7 (n=5) or Day 1 of Cycle 13 (n=1) showed an increase in the levels of antigen-specific CD8+ T cell frequency, assessed by pMHC staining. The mean CD8+ T cell frequency at the pre-boost timepoint (Day 1 of Cycle 6) was 0.87%, which increased to 1.4% at post-boost assessment (Day 1 of Cycle 8), showing a 60% increase in frequency of CD8+ T cells. In one of the CD8+ T cell responses, the boost dose at Day 1 of Cycle 13 led to a 4-fold increase in the frequency of CD8+ T cell response (from 0.12% to 0.48%). See FIG. 9 and Table 7 below. These data support administration of individualized RNA vaccine during a boost phase to further increase antigen-specific CD8+ T cell responses.

TABLE 7
Cancer types of Phase Ia/Ib patients

| | Neoantigen-specific | Neoantigen-specific |
|---|---|---|
| | CD8 T cells (%) at Cycle | CD8 T cells (%) at Cycle |
| Cancer type | 6, Day 1 (pre-boost) | 8, Day 1 (post-boost) |
| Triple Negative | 1.84 | 2.97 |
| Breast Cancer | | |
| Ovarian Cancer | 0.03 | 0.07 |
| Colon Cancer | 1.87 | 2.9 |
| Prostate Cancer | 0.38 | 0.71 |
| Cervical Cancer | 0.25 | 0.32 |

### Example 4: TCR Clonal Expansion as a Biomarker of Immune Response to RNA Vaccine as a Single Agent, or in a Combination with Checkpoint Blockade

T cell receptor sequencing (TCR-seq) can be utilized to assess the change in immune repertoire upon treatment. The nucleotide and amino acid sequences of each TCR can serve as natural molecular barcodes to track the presence and abundance of T cell clones at various stages of treatment. RNA vaccine as a single agent or in combination with a checkpoint blockade can boost the pre-existing or introduce de novo immune responses. These responses can be captured by TCR-seq. Based on the number of expanded TCR clones or/and their frequencies, the immune responders to the vaccine can be predicted. Machine-learning approaches can improve the prediction algorithms to identify immune responders based on the changes in T cell repertoire and frequency.

Statistical methods like binomial models based on a Fisher's exact test (DeWitt et al., J Virol 2015;89 (8): 4517-4526) or beta-binomial models (Rytlewski et al., PLOS One 2019; 14 (3): e0213684) can be used to compare the frequency of TCR clones at any time points relative to each other. TCR-seq data from on-treatment and post-treatment peripheral blood samples can be compared to the pre-treatment sample to capture the significantly expanded (SE) clones upon the administration of vaccine or/and checkpoint blockade.

### Study Design

TCR-seq from 50 patients of Phase Ia/Ib study, as described in the study design of Example 3 and Examples 1-5 of PCT/US2021/015710, were captured at pre-treatment (baseline) and post-vaccine time points: about 3 doses after RNA vaccine (3-Vax), about 6 doses after vaccine (6-Vax), about 8 doses after vaccine (8-Vax), and about 9 or 10 doses after vaccine (9/10-Vax). The immunogenicity of this fifty-patient cohort was already assessed with ELISpot; an IFN-γ release assay which is typically used in vaccine research to define vaccine efficacy by measuring the capacity to elicit T cell responses. Out of these 50 patients, 38 had at least one neoantigen-specific immune response against the RNA vaccine (i.e, ELISpot positive), and 12 had no immune response (i.e., ELISpot negative).

A beta-binomial statistical model was used to determine the number of significantly expanded clones at each post-vaccine time point relative to the baseline (padjusted <0.01; p values adjusted by Benjamini-Hochberg (bh) correction to control false discovery rate). See FIG. 10. The goal of this study was to assess the capability of TCR-seq as an alternative approach to predict immune response to RNA vaccine. Compared to ELISpot assay as described above, the proposed TCR-seq approach requires less input (volume of blood) and can have a faster turnaround time.

Table 8 below provides an overview of mixed cancer indications used in this fifty-patient cohort TCR-seq analysis.

TABLE 8
Cancer indications in phase 1a/1b patients used in TCR-seq study.

| Cancer Indication | Number of Patients |
|---|---|
| Non-small cell lung cancer | 9 |
| Melanoma | 6 |
| Renal Cell Carcinoma | 6 |
| Breast Cancer | 5 |
| Colorectal Cancer | 5 |
| Ovarian Cancer | 5 |
| Prostate Cancer | 5 |
| Urinary Bladder Cancer | 4 |
| Cervical Cancer | 2 |
| Bone Cancer | 2 |
| Head and Neck Squamous Cell Carcinoma | 1 |

### Results

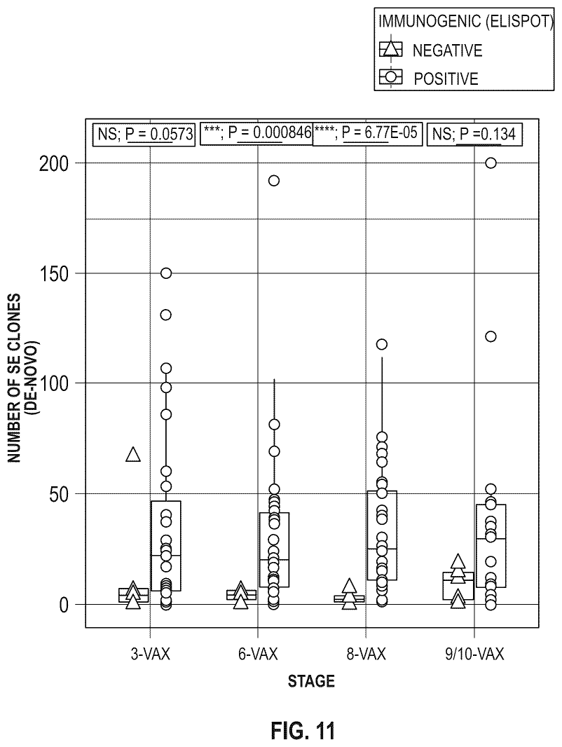

A clear signal was observed for the association of immunogenicity of patients identified by ELISpot assay and TCR-seq assay. The TCR clonal expansion can therefore serve as a biomarker to associate with ELISpot. Higher numbers of de novo significantly expanded (SE) clones were observed in immune responders to the vaccine as a single agent or in combination with checkpoint blockade. A response was identified as de novo if no TCR clone was detected in the pre-treatment sample. See FIG. 11. Even after 3 doses of vaccine (3-Vax), a difference in the number of de novo SE clones was noticed between immune responders to the vaccine (i.e., ELISpot positive) compared to patients with no immune response (i.e., ELISpot negative). However, this difference was not statistically significant (p-value=0.0573). This difference gained statistical power after 6 and 8 doses of vaccine (p-values of 0.000846 and 0.0000677, respectively).

Six doses after the vaccine can serve as an optimal time point to detect the immune response to the vaccine. With access to the TCR repertoire of peripheral blood at baseline (i.e., pre-treatment) and after 6 doses of vaccine (6-Vax), a predictive model can detect the immune response to the vaccine based on the number of significantly expanded clones. We identified a cutoff of 6 de novo significantly expanded (SE) clones at 6-Vax relative to the baseline as a measure to predict immunogenicity to the vaccine as a single agent or in a combination with checkpoint blockade in the current cohort. Patients with de novo SE TCR clones higher than 6 (SE >6) were predicted as immunogenic by this TCR-seq approach.

Among the 50 patients in this study, 39 of them had TCR data at 6-Vax: 9 ELISpot-negative patients and 30 ELISpot-positive patients. By using a 6 de novo SE TCR clone cutoff, the approach predicted all ELISpot-negative (i.e., no immune responders to the vaccine) and 80% of ELISpot-positive patients (i.e., immune responders to the vaccine). See Table 9. The TCR-seq based number of expanded clones have a specificity of 100% and a sensitivity of 80% in predicting immune responses to the vaccine based on ELISpot assay. Thus, this approach can substitute ELISpot for future clinical trials to assess the immune responses to vaccines in a more rapid and less labor-intensive fashion.

TABLE 9
TCR clonal expansion as a marker of immunogenicity.

| | The number | | | | |
|---|---|---|---|---|---|
| SE | of patients | ELISpot- | ELISpot- | ELISpot- | ELISpot- |
| cutoff at | with 6-Vax | negative | negative | positive | positive |
| 6-Vax | TCR data | out ↑ | in ↓ | out ↓ | in ↑ |
| 6 | ELISpot- | 9, 100% | 0, 0% | 6, 20% | 24, 80% |
| | negative | | | | |
| | (9) vs | | | | |
| | ELISpot- | | | | |
| | positive (30) | | | | |

SEQUENCES

All polynucleotide sequences are depicted in the 5′→3′ direction. All
polypeptide sequences are depicted in the N-terminal to C-terminal direction.
Anti-PDL1 antibody HVR-H1 sequence (SEQ ID NO: 1)
GFTFSDSWIH
Anti-PDL1 antibody HVR-H2 sequence (SEQ ID NO: 2)
AWISPYGGSTYYADSVKG
Anti-PDL1 antibody HVR-H3 sequence (SEQ ID NO: 3)
RHWPGGFDY
Anti-PDL1 antibody HVR-L1 sequence (SEQ ID NO: 4)
RASQDVSTAVA
Anti-PDL1 antibody HVR-L2 sequence (SEQ ID NO: 5)
SASFLYS
Anti-PDL1 antibody HVR-L3 sequence (SEQ ID NO: 6)
QQYLYHPAT
Anti-PDL1 antibody VH sequence (SEQ ID NO: 7)
EVOLVESGGGLVQPGGSLRLSCAASGFTFSDSWIHWVRQAPGKGLEWVAWISPYGGSTYYA
DSVKGRFTISADTSKNTAYLQMNSLRAEDTAVYYCARRHWPGGFDYWGQGTLVTVSS
Anti-PDL1 antibody VL sequence (SEQ ID NO: 8)
DIQMTQSPSSLSASVGDRVTITCRASQDVSTAVAWYQQKPGKAPKLLIYSASF
LYSGVPSRFSGSGSGTDFTLTISSLQPEDFATYYCQQYLYHPATFGQGTKVEIKR
Anti-PDL1 antibody heavy chain sequence (SEQ ID NO: 9)
EVQLVESGGGLVQPGGSLRLSCAASGFTFSDSWIHWVRQAPGKGLEWVAWISPYGGSTYYA
DSVKGRFTISADTSKNTAYLQMNSLRAEDTAVYYCARRHWPGGFDYWGQGTLVTVSSAST
KGPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYASTYRVVSVLTV
LHQDWLNGKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHEAL
HNHYTQKSLSLSPG
Anti-PDL1 antibody light chain sequence (SEQ ID NO: 10)
DIQMTQSPSSLSASVGDRVTITCRASQDVSTAVAWYQQKPGKAPKLLIYSASFLYSGVPSRFS
GSGSGTDFTLTISSLQPEDFATYYCQQYLYHPATFGQGTKVEIKRTVAAPSVFIFPPSDEQLKS
GTASVVCLLNNFYPREAKVQWKVDNALQSGNSQESVTEQDSKDSTYSLSSTLTLSKADYEK
HKVYACEVTHQGLSSPVTKSFNRGEC
Nivolumab heavy chain sequence (SEQ ID NO: 11)
QVQLVESGGGVVQPGRSLRLDCKASGITFSNSGMHWVRQAPGKGLEWVAVIWY
DGSKRYYADSVKGRFTISRDNSKNTLFLQMNSLRAEDTAVYYCATNDDYWGQGTLVTVSS
ASTKGPSVFPLAPCSRSTSESTAALGCLVKDYFPEPVTVSWNSGALTSGVHTFPAVLQSSGLY
SLSSVVTVPSSSLGTKTYTCNVDHKPSNTKVDKRVESKYGPPCPPCPAPEFLGGPSVFLFPPKP
KDTLMISRTPEVTCVVVDVSQEDPEVQFNWYVDGVEVHNAKTKPREEQFNSTYRVVSVLTV
LHQDWLNGKEYKCKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEGNVFSCSVMHEAL
HNHYTQKSLSLSLG
Nivolumab light chain sequence (SEQ ID NO: 12)
EIVLTQSPATLSLSPGERATLSCRASQSVSSYLAWYQQKPGQAPRLLIYDASNRAT
GIPARFSGSGSGTDFTLTISSLEPEDFAVYYCQQSSNWPRTFGQGTKVEIKRTVAAPSVFIFPPS
DEQLKSGTASVVCLLNNFYPREAKVQWKVDNALQSGNSQESVTEQDSKDSTYSLSSTLTLSK
ADYEKHKVYACEVTHQGLSSPVTKSFNRGEC
Pembrolizumab heavy chain sequence (SEQ ID NO: 13)
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRDYRFDMGFDYW
GQGTTVTVSSASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVESKYGPPCPPCP
APEFLGGPSVFLFPPKPKDTLMISRTPEVTCVVVDVSQEDPEVQFNWYVDGVEVHNAKTK
PREEQFNSTYRVVSVLTVLHQDWLNGKEYKCKVSNKGLPSSIEKTISKAK
GQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLG
Pembrolizumab light chain sequence (SEQ ID NO: 14)
EIVLTQSPATLSLSPGERATLSCRASKGVSTSGYSYLHWYQQKPGQAPRLLIYLASYLES
GVPARFSGSGSGTDFTLTISSLEPEDFAVYYCQHSRDLPLTFGGGTKVEIKRTVAAPSVF
IFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
Avelumab heavy chain sequence (SEQ ID NO: 15)
EVQLLESGGGLVQPGGSLRLSCAASGFTFSSYIMMWVRQAPGKGLEWVSSIYPSGGITFYAD
TVKGRFTISRDNSKNTLYLQMNSLRAEDTAVYYCARIKLGTVTTVDYWGQGTLVTVSSAST
KGPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEAL
HNHYTQKSLSLSPG
Avelumab light chain sequence (SEQ ID NO: 16)
QSALTQPASVSGSPGQSITISCTGTSSDVGGYNYVSWYQQHPGKAPKLMIYDVSNRPSGVSN
RFSGSKSGNTASLTISGLQAEDEADYYCSSYTSSSTRVFGTGTKVTVLGQPKANPTVTLFPPSS
EELQANKATLVCLISDFYPGAVTVAWKADGSPVKAGVETTKPSKQSNNKYAASSYLSLTPEQ
WKSHRSYSCQVTHEGSTVEKTVAPTECS
Durvalumab heavy chain sequence (SEQ ID NO: 17)
EVQLVESGGGLVQPGGSLRLSCAASGFTFSRYWMSWVRQAPGKGLEWVANIKQDGSEKYY
VDSVKGRFTISRDNAKNSLYLQMNSLRAEDTAVYYCAREGGWFGELAFDYWGQGTLVTVS
SASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPCPAPEFEGGPSVFLF
PPKPKDTLMISRTPEVTCVVVDVSHEDPEVKFNWYVDGVEVHNAKTKPREEQYNSTYRVVS
VLTVLHQDWLNGKEYKCKVSNKALPASIEKTISKAKGQPREPQVYTLPPSREEMTKNQVSLT
CLVKGFYPSDIAVEWESNGQPENNYKTTPPVLDSDGSFFLYSKLTVDKSRWQQGNVFSCSV
MHEALHNHYTQKSLSLSPG
Durvalumab light chain sequence (SEQ ID NO: 18)
EIVLTQSPGTLSLSPGERATLSCRASQRVSSSYLAWYQQKPGQAPRLLIYDASSRATGIPDRFS
GSGSGTDFTLTISRLEPEDFAVYYCQQYGSLPWTFGQGTKVEIKRTVAAPSVFIFPPSDEQLKS
GTASVVCLLNNFYPREAKVQWKVDNALQSGNSQESVTEQDSKDSTYSLSSTLTLSKADYEK
HKVYACEVTHQGLSSPVTKSFNRGEC
Full ICV RNA 5′ constant sequence (SEQ ID NO: 19)
GGCGAACUAGUAUUCUUCUGGUCCCCACAGACUCAGAGAGAACCCGCCACCAUGAGAG
UGAUGGCCCCCAGAACCCUGAUCCUGCUGCUGUCUGGCGCCCUGGCCCUGACAGAGAC
AUGGGCCGGAAGC
Full ICV RNA 3′ constant sequence (SEQ ID NO: 20)
AUCGUGGGAAUUGUGGCAGGACUGGCAGUGCUGGCCGUGGUGGUGAUCGGAGCCGUG
GUGGCUACCGUGAUGUGCAGACGGAAGUCCAGCGGAGGCAAGGGCGGCAGCUACAGC
CAGGCCGCCAGCUCUGAUAGCGCCCAGGGCAGCGACGUGUCACUGACAGCCUAGUAAC
UCGAGCUGGUACUGCAUGCACGCAAUGCUAGCUGCCCCUUUCCCGUCCUGGGUACCCC
GAGUCUCCCCCGACCUCGGGUCCCAGGUAUGCUCCCACCUCCACCUGCCCCACUCACCA
CCUCUGCUAGUUCCAGACACCUCCCAAGCACGCAGCAAUGCAGCUCAAAACGCUUAGC
CUAGCCACACCCCCACGGGAAACAGCAGUGAUUAACCUUUAGCAAUAAACGAAAGUUU
AACUAAGCUAUACUAACCCCAGGGUUGGUCAAUUUCGUGCCAGCCACACCGAGACCUG
GUCCAGAGUCGCUAGCCGCGUCGCU
Full ICV Kozak RNA (SEQ ID NO: 21)
GGCGAACUAGUAUUCUUCUGGUCCCCACAGACUCAGAGAGAACCCGCCACC
Full ICV Kozak DNA (SEQ ID NO: 22)
GGCGAACTAGTATTCTTCTGGTCCCCACAGACTCAGAGAGAACCCGCCACC
short Kozak RNA (SEQ ID NO: 23)
UUCUUCUGGUCCCCACAGACUCAGAGAGAACCCGCCACC
short Kozak DNA (SEQ ID NO: 24)
TTCTTCTGGTCCCCACAGACTCAGAGAGAACCCGCCACC
sec RNA (SEQ ID NO: 25)
AUGAGAGUGAUGGCCCCCAGAACCCUGAUCCUGCUGCUGUCUGGCGCCCUGGCCCUGA
CAGAGACAUGGGCCGGAAGC
sec DNA (SEQ ID NO: 26)
ATGAGAGTGATGGCCCCCAGAACCCTGATCCTGCTGCTGTCTGGCGCCCTGGCCCTGACA
GAGACATGGGCCGGAAGC
sec protein (SEQ ID NO: 27)
MRVMAPRTLILLLSGALALTETWAGS
MITD RNA (SEQ ID NO: 28)
AUCGUGGGAAUUGUGGCAGGACUGGCAGUGCUGGCCGUGGUGGUGAUCGGAGCCGUG
GUGGCUACCGUGAUGUGCAGACGGAAGUCCAGCGGAGGCAAGGGCGGCAGCUACAGC
CAGGCCGCCAGCUCUGAUAGCGCCCAGGGCAGCGACGUGUCACUGACAGCC
MITD DNA (SEQ ID NO: 29)
ATCGTGGGAATTGTGGCAGGACTGGCAGTGCTGGCCGTGGTGGTGATCGGAGCCGTGGT
GGCTACCGTGATGTGCAGACGGAAGTCCAGCGGAGGCAAGGGCGGCAGCTACAGCCAG
GCCGCCAGCTCTGATAGCGCCCAGGGCAGCGACGTGTCACTGACAGCC
MITD protein (SEQ ID NO: 30)
IVGIVAGLAVLAVVVIGAVVATVMCRRKSSGGKGGSYSQAASSDSAQGSDVSLTA
Full ICV FI RNA (SEQ ID NO: 31)
CUCGAGCUGGUACUGCAUGCACGCAAUGCUAGCUGCCCCUUUCCCGUCCUGGGUACCC
CGAGUCUCCCCCGACCUCGGGUCCCAGGUAUGCUCCCACCUCCACCUGCCCCACUCACC
ACCUCUGCUAGUUCCAGACACCUCCCAAGCACGCAGCAAUGCAGCUCAAAACGCUUAG
CCUAGCCACACCCCCACGGGAAACAGCAGUGAUUAACCUUUAGCAAUAAACGAAAGUU
UAACUAAGCUAUACUAACCCCAGGGUUGGUCAAUUUCGUGCCAGCCACACCGAGACCU
GGUCCAGAGUCGCUAGCCGCGUCGCU
Full ICV FI DNA (SEQ ID NO: 32)
CTGGTACTGCATGCACGCAATGCTAGCTGCCCCTTTCCCGTCCTGGGTACCCCGAGTCTC
CCCCGACCTCGGGTCCCAGGTATGCTCCCACCTCCACCTGCCCCACTCACCACCTCTGCT
AGTTCCAGACACCTCCCAAGCACGCAGCAATGCAGCTCAAAACGCTTAGCCTAGCCACA
CCCCCACGGGAAACAGCAGTGATTAACCTTTAGCAATAAACGAAAGTTTAACTAAGCTA
TACTAACCCCAGGGTTGGTCAATTTCGTGCCAGCCACACCGAGACCTGGTCCAGAGTCGC
TAGCCGCGTCGCT
F element RNA (SEQ ID NO: 33)
CUGGUACUGCAUGCACGCAAUGCUAGCUGCCCCUUUCCCGUCCUGGGUACCCCGAGUC
UCCCCCGACCUCGGGUCCCAGGUAUGCUCCCACCUCCACCUGCCCCACUCACCACCUCU
GCUAGUUCCAGACACCUCC
F element DNA (SEQ ID NO: 34)
CTGGTACTGCATGCACGCAATGCTAGCTGCCCCTTTCCCGTCCTGGGTACCCCGAGTCTC
CCCCGACCTCGGGTCCCAGGTATGCTCCCACCTCCACCTGCCCCACTCACCACCTCTGCT
AGTTCCAGACACCTCC
I element RNA (SEQ ID NO: 35)
CAAGCACGCAGCAAUGCAGCUCAAAACGCUUAGCCUAGCCACACCCCCACGGGAAACA
GCAGUGAUUAACCUUUAGCAAUAAACGAAAGUUUAACUAAGCUAUACUAACCCCAGG
GUUGGUCAAUUUCGUGCCAGCCACACCG
I element DNA (SEQ ID NO: 36)
CAAGCACGCAGCAATGCAGCTCAAAACGCTTAGCCTAGCCACACCCCCACGGGAAACAG
CAGTGATTAACCTTTAGCAATAAACGAAAGTTTAACTAAGCTATACTAACCCCAGGGTTG
GTCAATTTCGTGCCAGCCACACCG
linker RNA (SEQ ID NO: 37)
GGCGGCUCUGGAGGAGGCGGCUCCGGAGGC
linker DNA (SEQ ID NO: 38)
GGCGGCTCTGGAGGAGGCGGCTCCGGAGGC
linker protein (SEQ ID NO: 39)
GGSGGGGSGG
Full ICV DNA 5′ constant sequence (SEQ ID NO: 40)
GGCGAACTAGTATTCTTCTGGTCCCCACAGACTCAGAGAGAACCCGCCACCATGAGAGT
GATGGCCCCCAGAACCCTGATCCTGCTGCTGTCTGGCGCCCTGGCCCTGACAGAGACATG
GGCCGGAAGC
Full ICV DNA 3′ constant sequence (SEQ ID NO: 41)
ATCGTGGGAATTGTGGCAGGACTGGCAGTGCTGGCCGTGGTGGTGATCGGAGCCGTGGT
GGCTACCGTGATGTGCAGACGGAAGTCCAGCGGAGGCAAGGGCGGCAGCTACAGCCAG
GCCGCCAGCTCTGATAGCGCCCAGGGCAGCGACGTGTCACTGACAGCCTAGTAACTCGA
GCTGGTACTGCATGCACGCAATGCTAGCTGCCCCTTTCCCGTCCTGGGTACCCCGAGTCT
CCCCCGACCTCGGGTCCCAGGTATGCTCCCACCTCCACCTGCCCCACTCACCACCTCTGCT
AGTTCCAGACACCTCCCAAGCACGCAGCAATGCAGCTCAAAACGCTTAGCCTAGCCACA
CCCCCACGGGAAACAGCAGTGATTAACCTTTAGCAATAAACGAAAGTTTAACTAAGCTA
TACTAACCCCAGGGTTGGTCAATTTCGTGCCAGCCACACCGAGACCTGGTCCAGAGTCGC
TAGCCGCGTCGCT
Full ICV RNA with 5′ GG from cap (SEQ ID NO: 42)
GGGGCGAACU AGUAUUCUUC UGGUCCCCAC AGACUCAGAG AGAACCCGCC
ACCAUGAGAG UGAUGGCCCC CAGAACCCUG AUCCUGCUGC UGUCUGGCGC
CCUGGCCCUG ACAGAGACAU GGGCCGGAAG CNAUCGUGGGA AUUGUGGCAG
GACUGGCAGU GCUGGCCGUG GUGGUGAUCG GAGCCGUGGU GGCUACCGUG
AUGUGCAGAC GGAAGUCCAG CGGAGGCAAG GGCGGCAGCU ACAGCCAGGC
CGCCAGCUCU GAUAGCGCCC AGGGCAGCGA CGUGUCACUG ACAGCCUAGU
AACUCGAGCU GGUACUGCAU GCACGCAAUG CUAGCUGCCC CUUUCCCGUC
CUGGGUACCC CGAGUCUCCC CCGACCUCGG GUCCCAGGUA UGCUCCCACC
UCCACCUGCC CCACUCACCA CCUCUGCUAG UUCCAGACAC CUCCCAAGCA
CGCAGCAAUG CAGCUCAAAA CGCUUAGCCU AGCCACACCC CCACGGGAAA
CAGCAGUGAU UAACCUUUAG CAAUAAACGA AAGUUUAACU AAGCUAUACU
AACCCCAGGG UUGGUCAAUU UCGUGCCAGC CACACCGAGA CCUGGUCCAG
AGUCGCUAGC CGCGUCGCUA AAAAAAAAAA AAAAAAAAAA AAAAAAAAAA
AAAAAAAAAA AAAAAAAAAA AAAAAAAAAA AAAAAAAAAA AAAAAAAAAA
AAAAAAAAAA AAAAAAAAAA AAAAAAAAAA AAAAAAAAA

What is claimed is:

1. A method for treating a pancreatic cancer tumor in a human patient in need thereof, comprising administering to the patient:
(a) an individualized RNA vaccine comprising one or more polynucleotides encoding one or more neoepitopes resulting from cancer-specific somatic mutations present in a pancreatic cancer tumor specimen obtained from the patient,
(b) a PD-1 axis binding antagonist, and
(c) a chemotherapeutic treatment;
wherein the RNA vaccine, the PD-1 axis binding antagonist, and the chemotherapeutic treatment are administered to the patient during a priming phase, a chemotherapy phase after the priming phase, and a boost phase after the chemotherapy phase, wherein:
(i) the priming phase comprises administering to the patient at least one dose of the RNA vaccine and at least one dose of the PD-1 axis binding antagonist,
(ii) the chemotherapy phase comprises administering to the patient the chemotherapeutic treatment, and
(iii) the boost phase comprises administering to the patient at least one dose of the RNA vaccine and at least one dose of the PD-1 axis binding antagonist.

2. The method of claim 1, wherein the pancreatic cancer tumor is a pancreatic ductal adenocarcinoma (PDAC) tumor.

3. The method of claim 1 or claim 2, wherein the pancreatic cancer tumor is resectable.

4. The method of claim 3, wherein the priming phase begins at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 weeks, at least about 14 weeks, or at least about 15 weeks after resection of the pancreatic cancer tumor from the patient.

5. The method of claim 3, wherein the priming phase begins between about 6 weeks and about 12 weeks after resection of the pancreatic cancer tumor from the patient.

6. The method of any one of claims 1-5, wherein the priming phase comprises administering one dose of the PD-1 axis binding antagonist.

7. The method of claim 6, wherein the priming phase comprises administering the PD-1 axis binding antagonist on day 1 of week 3 of the priming phase.

8. The method of any one of claims 1-5, wherein the priming phase comprises administering at least two doses of the PD-1 axis binding antagonist.

9. The method of any one of claims 1-5 and 8, wherein the priming phase comprises administering the PD-1 axis binding antagonist once every four weeks.

10. The method of claim 9, wherein the priming phase comprises administering the PD-1 axis binding antagonist on day 1 of week 1 of the priming phase and every four weeks thereafter.

11. The method of any one of claims 1-5 and 8-10, wherein the priming phase comprises administering two doses of the PD-1 axis binding antagonist.

12. The method of claim 11, wherein the priming phase comprises administering the PD-1 axis binding antagonist on day 1 of week 1 and on day 1 of week 5 of the priming phase.

13. The method of any one of claims 1-12, wherein the priming phase comprises administering any of 2, 3, 4, 5, 6, 7, or 8 doses of the RNA vaccine.

14. The method of claim 13, wherein the priming phase comprises administering 2 or 3 doses of the RNA vaccine.

15. The method of any one of claims 1-13, wherein the priming phase comprises administering between 6 and 8 doses of the RNA vaccine, or up to six doses of the RNA vaccine.

16. The method of claim 15, wherein the priming phase comprises administering 6 doses of the RNA vaccine.

17. The method of any one of claims 1-16, wherein the priming phase comprises administering the RNA vaccine once per week.

18. The method of claim 17, wherein the priming phase comprises administering the RNA vaccine on day 1 of week 1 of the priming phase and once per week thereafter.

19. The method of any one of claims 1-18, wherein the priming phase comprises administering six doses of the RNA vaccine.

20. The method of claim 19, wherein the priming phase comprises administering the RNA vaccine on day 1 of weeks 1, 2, 3, 4, 5, and 6 of the priming phase.

21. The method of any one of claims 1-20, wherein each dose of the PD-1 axis binding antagonist administered to the patient during the priming phase is administered on the same day as administration of a dose of the RNA vaccine.

22. The method of any one of claims 1-21, wherein the priming phase comprises six weeks.

23. The method of claim 22, wherein the RNA vaccine is administered on day 1 of weeks 1, 2, 3, 4, 5, and 6 of the priming phase, and the PD-1 axis binding antagonist is administered on day 1 of week 3 of the priming phase.

24. The method of claim 22, wherein the RNA vaccine is administered on day 1 of weeks 1, 2, 3, 4, 5, and 6 of the priming phase, and the PD-1 axis binding antagonist is administered on day 1 of weeks 1 and 5 of the priming phase.

25. The method of any one of claims 1-24, wherein the chemotherapy phase comprises administering the chemotherapeutic treatment for at least about 8 weeks, at least about 9 weeks, at least about 10 weeks, at least about 11 weeks, at least about 12 weeks, at least about 13 weeks, at least about 14 weeks, at least about 15 weeks, at least about 16 weeks, at least about 17 weeks, at least about 18 weeks, at least about 19 weeks, at least about 20 weeks, at least about 21 weeks, at least about 22 weeks, at least about 23 weeks, at least about 24 weeks, at least about 25 weeks, at least about 26 weeks, at least about 27 weeks, at least about 28 weeks, at least about 29 weeks, at least about 30 weeks, or more.

26. The method of any one of claims 1-25, wherein the chemotherapy phase comprises administering the chemotherapeutic treatment for 23 weeks.

27. The method of any one of claims 1-26, wherein the chemotherapeutic treatment is administered once every two weeks.

28. The method of claim 27, wherein the chemotherapy phase comprises administering the chemotherapeutic treatment on day 1 of week 1 of the chemotherapy phase and every two weeks thereafter.

29. The method of any one of claims 1-28, wherein the chemotherapy phase comprises administering at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, or at least 24, or more, administrations of the chemotherapeutic treatment.

30. The method of claim 29, wherein the chemotherapy phase comprises administering 12 administrations of the chemotherapeutic treatment.

31. The method of any one of claims 1-30, wherein the chemotherapy phase comprises 24 weeks.

32. The method of any one of claims 1-31, wherein the chemotherapy phase comprises administering the chemotherapeutic treatment on day 1 of weeks 1, 3, 5, 7, 9, 11, 13, 15, 17, 19, 21, and 23 of the chemotherapy phase.

33. The method of any one of claims 1-32, wherein the chemotherapy phase begins at least about 1 week, at least about 2 weeks, at least about 3 weeks, or at least about 4 weeks after the end of the priming phase and after the last administration of the RNA vaccine.

34. The method of any one of claims 1-32, wherein the chemotherapy phase begins no later than week 9, timing starting with week 1 of the priming phase.

35. The method of claim 34, wherein the priming phase comprises six weeks, and wherein the chemotherapy phase begins no later than week 9, timing starting with week 1 of the priming phase.

36. The method of any one of claims 1-35, wherein the priming phase comprises six weeks, and wherein the chemotherapy phase comprises administering the chemotherapeutic treatment starting on day 1 of week 7 and every two weeks thereafter, timing starting with week 1 of the priming phase.

37. The method of claim 36, wherein the chemotherapy phase comprises administering 12 administrations of the chemotherapeutic treatment.

38. The method of claim 37, wherein the chemotherapy phase comprises administering the chemotherapeutic treatment on day 1 of weeks 7, 9, 11, 13, 15, 17, 19, 21, 23, 25, 27, and 29, timing starting with week 1 of the priming phase.

39. The method of any one of claims 1-38, wherein the boost phase comprises administering 2, 3, 4, 5, 6, 7, or 8 doses of the RNA vaccine.

40. The method of any one of claims 1-39, wherein the boost phase comprises administering 2, 3, 4, 5, 6, 7, or 8 doses of the PD-1 axis binding antagonist.

41. The method of any one of claims 1-40, wherein the boost phase comprises administering 6 doses of the PD-1 axis binding antagonist and 6 doses of the RNA vaccine.

42. The method of any one of claims 1-41, wherein the boost phase comprises administering the PD-1 axis binding antagonist and the RNA vaccine once every four weeks.

43. The method of any one of claims 1-42, wherein the boost phase comprises administering the PD-1 axis binding antagonist on day 1 of week 1 of the boost phase and every four weeks thereafter.

44. The method of any one of claims 1-43, wherein the boost phase comprises administering the RNA vaccine on day 1 of week 1 of the boost phase and every four weeks thereafter.

45. The method of any one of claims 1-44, wherein administrations of the RNA vaccine and the PD-1 axis binding antagonist during the boost phase occur on the same day.

46. The method of claim 45, wherein the boost phase comprises administering the PD-1 axis binding antagonist and the RNA vaccine on day 1 of week 1 of the boost phase and every four weeks thereafter.

47. The method of any one of claims 1-46, wherein the boost phase comprises 21 weeks.

48. The method of any one of claims 1-47, wherein the RNA vaccine and the PD-1 axis binding antagonist are administered on day 1 of weeks 1, 5, 9, 13, 17, and 21 of the boost phase.

49. The method of any one of claims 1-48, wherein the boost phase begins at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 weeks, at least about 14 weeks, or at least about 15 weeks after the end of the chemotherapy phase.

50. The method of any one of claims 1-48, wherein the boost phase begins up to about 12 weeks after the end of the chemotherapy phase, optionally up to about 12 weeks after the last administration of the chemotherapeutic treatment.

51. The method of any one of claims 1-48, wherein the boost phase begins:
between about 3 weeks to about 12 weeks after the end of the chemotherapy phase, optionally between about 3 weeks to about 12 weeks after the last administration of the chemotherapeutic treatment; or
about three weeks or about four weeks after the end of the chemotherapy phase, optionally about three weeks or about four weeks after the last administration of the chemotherapeutic treatment.

52. The method of any one of claims 1-48, wherein the boost phase begins on week 27, timing starting with week 1 of the chemotherapy phase.

53. The method of any one of claims 1-48, wherein the boost phase begins on week 33, timing starting with week 1 of the priming phase.

54. The method of claim 53, wherein the boost phase comprises administering the RNA vaccine and the PD-1 axis binding antagonist on day 1 of week 33 and every four weeks thereafter, timing starting with week 1 of the priming phase.

55. The method of claim 54, wherein the RNA vaccine and the PD-1 axis binding antagonist are administered for six administrations during the boost phase.

56. The method of claim 55, wherein the boost phase comprises administering the RNA vaccine and the PD-1 axis binding antagonist on day 1 of weeks 33, 37, 41, 45, 49, and 53, timing starting with week 1 of the priming phase.

57. The method of any one of claims 1-7, 13-23 and 25-56, wherein:
(a) the priming phase comprises administering the RNA vaccine on day 1 of weeks 1, 2, 3, 4, 5, and 6 of the priming phase, and the PD-1 axis binding antagonist on day 1 of week 3 of the priming phase;
(b) the chemotherapy phase comprises administering the chemotherapeutic treatment on day 1 of weeks 7, 9, 11, 13, 15, 17, 19, 21, 23, 25, 27, and 29, timing starting with week 1 of the priming phase; and
(c) the boost phase comprises administering the RNA vaccine and the PD-1 axis binding antagonist on day 1 of weeks 33, 37, 41, 45, 49, and 53, timing starting with week 1 of the priming phase.

58. The method of any one of claims 1-5, 8-22, and 24-56, wherein:
(a) the priming phase comprises administering the RNA vaccine on day 1 of weeks 1, 2, 3, 4, 5, and 6 of the priming phase, and the PD-1 axis binding antagonist on day 1 of weeks 1 and 5 of the priming phase;
(b) the chemotherapy phase comprises administering the chemotherapeutic treatment on day 1 of weeks 7, 9, 11, 13, 15, 17, 19, 21, 23, 25, 27, and 29, timing starting with week 1 of the priming phase; and
(c) the boost phase comprises administering the RNA vaccine and the PD-1 axis binding antagonist on day 1 of weeks 33, 37, 41, 45, 49, and 53, timing starting with week 1 of the priming phase.

59. The method of claim 57 or claim 58, wherein the priming phase begins between about 6 weeks and about 12 weeks after resection of the pancreatic cancer tumor from the patient.

60. The method of any one of claims 1-59, wherein the PD-1 axis binding antagonist is a PD-1 binding antagonist.

61. The method of claim 60, wherein the PD-1 binding antagonist is an anti-PD-1 antibody.

62. The method of claim 61, wherein the anti-PD-1 antibody is nivolumab or pembrolizumab.

63. The method of any one of claims 1-59, wherein the PD-1 axis binding antagonist is a PD-L1 binding antagonist.

64. The method of claim 63, wherein the PD-L1 binding antagonist is an anti-PD-L1 antibody.

65. The method of claim 64, wherein the anti-PD-L1 antibody is avelumab or durvalumab.

66. The method of claim 64, wherein the anti-PD-L1 antibody comprises:
(a) a heavy chain variable region (VH) that comprises an HVR-H1 comprising an amino acid sequence GFTFSDSWIH (SEQ ID NO:1), an HVR-H2 comprising an amino acid sequence AWISPYGGSTYYADSVKG (SEQ ID NO:2), and HVR-H3 comprising an amino acid sequence RHWPGGFDY (SEQ ID NO:3), and
(b) a light chain variable region (VL) that comprises an HVR-L1 comprising an amino acid sequence RASQDVSTAVA (SEQ ID NO:4), an HVR-L2 comprising an amino acid sequence SASFLYS (SEQ ID NO:5), and an HVR-L3 comprising an amino acid sequence QQYLYHPAT (SEQ ID NO:6).

67. The method of claim 64, wherein the anti-PD-L1 antibody comprises a heavy chain variable region (VH) comprising an amino acid sequence of SEQ ID NO:7 and a light chain variable region (VL) comprising an amino acid sequence of SEQ ID NO:8.

68. The method of claim 64, wherein the anti-PD-L1 antibody is atezolizumab.

69. The method of any one of claims 1-68, wherein the PD-1 axis binding antagonist is administered intravenously to the patient.

70. The method of any one of claims 64-69, wherein the anti-PD-L1 antibody is administered to the patient at a dose of about 1200 mg or about 1680 mg.

71. The method of claim 70, wherein the anti-PD-L1 antibody is atezolizumab, and the atezolizumab is administered intravenously to the patient at a dose of about 1680 mg.

72. The method of any one of claims 1-71, wherein the chemotherapeutic treatment comprises one or more of gemcitabine, leucovorin, 5-fluorouracil, capecitabine, irinotecan, liposomal irinotecan, a platinum-based chemotherapeutic agent, a taxane, and any combination thereof.

73. The method of claim 72, wherein the platinum-based chemotherapeutic agent is cisplatin, oxaliplatin, or both.

74. The method of claim 72 or claim 73, wherein the taxane is paclitaxel, docetaxel, albumin-bound paclitaxel, or any combination thereof.

75. The method of any one of claims 1-72, wherein the chemotherapeutic treatment comprises leucovorin, 5-fluorouracil, irinotecan, and oxaliplatin.

76. The method of any one of claims 1-72, wherein the chemotherapeutic treatment is a FOLFIRINOX treatment or an mFOLFIRINOX treatment.

77. The method of any one of claims 1-72, wherein the chemotherapeutic treatment comprises:
oxaliplatin at a dose of about 85 mg/m²;
leucovorin at a dose of about 400 mg/m²;
irinotecan at a dose of about 150 mg/m²; and/or
5-fluorouracil at a dose of about 2400 mg/m².

78. The method of any one of claims 1-77, wherein the chemotherapeutic treatment is administered intravenously to the patient.

79. The method of any one of claims 1-78, wherein the RNA vaccine comprises one or more polynucleotides encoding 5-20 or 10⁻²⁰ neoepitopes resulting from cancer-specific somatic mutations present in the tumor specimen.

80. The method of any one of claims 1-79, wherein the one or more polynucleotides of the RNA vaccine are formulated with one or more lipids.

81. The method of claim 80, wherein the one or more polynucleotides of the RNA vaccine and the one or more lipids form a lipid nanoparticle.

82. The method of claim 80, wherein the one or more polynucleotides of the RNA vaccine and the one or more lipids form a lipoplex.

83. The method of claim 81 or claim 82, wherein the lipid nanoparticle or lipoplex comprises one or more lipids that form a multilamellar structure that encapsulates the one or more polynucleotides of the RNA vaccine.

84. The method of claim 83, wherein the one or more lipids comprise at least one cationic lipid and at least one helper lipid.

85. The method of claim 83, wherein the one or more lipids comprise (R)-N,N,N-trimethyl-2,3-dioleyloxy-1-propanaminium chloride (DOTMA) and 1,2-dioleoyl-sn-glycero-3-phosphoethanolamine (DOPE).

86. The method of claim 85, wherein at physiological pH the overall charge ratio of positive charges to negative charges of the lipid nanoparticle or lipoplex is 1.3:2 (0.65).

87. The method of any one of claims 1-86, wherein the one or more polynucleotides of the RNA vaccine are RNA molecules, optionally messenger RNA molecules.

88. The method of any one of claims 1-87, wherein the RNA vaccine is administered to the patient at a dose of about 15 μg, about 21 μg, about 21.3 μg, about 25 μg, about 38 μg, or about 50 μg.

89. The method of claim 88, wherein the RNA vaccine is administered to the patient at a dose of about 25 μg.

90. The method of any one of claims 1-89, wherein the RNA vaccine is administered intravenously to the patient.

91. The method of any one of claims 1-90, wherein the RNA vaccine comprises an RNA molecule comprising, in the 5′→3′ direction:
(1) a 5′ cap;
(2) a 5′ untranslated region (UTR);
(3) a polynucleotide sequence encoding a secretory signal peptide;
(4) a polynucleotide sequence encoding the one or more neoepitopes resulting from cancer-specific somatic mutations present in the tumor specimen;
(5) a polynucleotide sequence encoding at least a portion of a transmembrane and cytoplasmic domain of a major histocompatibility complex (MHC) molecule;
(6) a 3′ UTR comprising:
(a) a 3′ untranslated region of an Amino-Terminal Enhancer of Split (AES) mRNA or a fragment thereof; and
(b) non-coding RNA of a mitochondrially encoded 12S RNA or a fragment thereof; and
(7) a poly(A) sequence.

92. The method of claim 91, wherein the RNA molecule further comprises a polynucleotide sequence encoding an amino acid linker; wherein the polynucleotide sequences encoding the amino acid linker and a first of the one or more neoepitopes form a first linker-neoepitope module; and
wherein the polynucleotide sequences forming the first linker-neoepitope module are between the polynucleotide sequence encoding the secretory signal peptide and the polynucleotide sequence encoding the at least portion of the transmembrane and cytoplasmic domain of the MHC molecule in the 5′→3′ direction.

93. The method of claim 92, wherein the amino acid linker comprises the sequence
(SEQ ID NO: 39)
GGSGGGGSGG.

94. The method of claim 92, wherein the polynucleotide sequence encoding the amino acid linker comprises the sequence GGCGGCUCUGGAGGAGGCGGCUCCGGAGGC (SEQ ID NO:37).

95. The method of any one of claims 92-94, wherein the RNA molecule further comprises, in the 5′→3′ direction: at least a second linker-neoepitope module, wherein the at least second linker-neoepitope module comprises a polynucleotide sequence encoding an amino acid linker and a polynucleotide sequence encoding a neoepitope; wherein the polynucleotide sequences forming the second linker-neoepitope module are between the polynucleotide sequence encoding the neoepitope of the first linker-neoepitope module and the polynucleotide sequence encoding the at least portion of the transmembrane and cytoplasmic domain of the MHC molecule in the 5′→3′ direction; and wherein the neoepitope of the first linker-neoepitope module is different from the neoepitope of the second linker-neoepitope module.

96. The method of claim 95, wherein the RNA molecule comprises 5 linker-neoepitope modules, and wherein the 5 linker-neoepitope modules each encode a different neoepitope.

97. The method of claim 95, wherein the RNA molecule comprises 10 linker-neoepitope modules, and wherein the 10 linker-neoepitope modules each encode a different neoepitope.

98. The method of claim 95, wherein the RNA molecule comprises 20 linker-neoepitope modules, and wherein the 20 linker-neoepitope modules each encode a different neoepitope.

99. The method of any one of claims 91-98, wherein the RNA molecule further comprises a second polynucleotide sequence encoding an amino acid linker, wherein the second polynucleotide sequence encoding the amino acid linker is between the polynucleotide sequence encoding the neoepitope that is most distal in the 3′ direction and the polynucleotide sequence encoding the at least portion of the transmembrane and cytoplasmic domain of the MHC molecule.

100. The method of any one of claims 91-99, wherein the 5′ cap comprises a DI diastereoisomer of the structure:

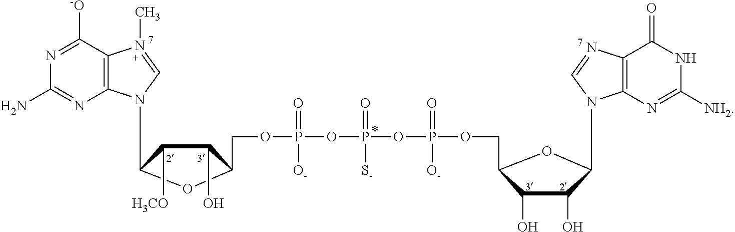

101. The method of any one of claims 91-100, wherein the 5′ UTR comprises the sequence
(SEQ ID NO: 23)
UUCUUCUGGUCCCCACAGACUCAGAGAGAACCCGCCACC.

102. The method of any one of claims 91-100, wherein the 5′ UTR comprises the sequence
(SEQ ID NO: 21)
GGCGAACUAGUAUUCUUCUGGUCCCCACAGACUCAGAGAGAACCCGCC
ACC.

103. The method of any one of claims 91-102, wherein the secretory signal peptide comprises the amino acid sequence MRVMAPRTLILLLSGALALTETWAGS (SEQ ID NO:27).

104. The method of any one of claims 91-102, wherein the polynucleotide sequence encoding the secretory signal peptide comprises the sequence
(SEQ ID NO: 25)
AUGAGAGUGAUGGCCCCCAGAACCCUGAUCCUGCUGCUGUCUGGCGCC
CUGGCCCUGACAGAGACAUGGGCCGGAAGC.

105. The method of any one of claims 91-104, wherein the at least portion of the transmembrane and cytoplasmic domain of the MHC molecule comprises the amino acid sequence
(SEQ ID NO: 30)
IVGIVAGLAVLAVVVIGAVVATVMCRRKSSGGKGGSYSQAASSDSAQG
SDVSLTA.

106. The method of any one of claims 91-104, wherein the polynucleotide sequence encoding the at least portion of the transmembrane and cytoplasmic domain of the MHC molecule comprises the sequence
(SEQ ID NO: 28)
AUCGUGGGAAUUGUGGCAGGACUGGCAGUGCUGGCCGUGGUGGUGAUC
GGAGCCGUGGUGGCUACCGUGAUGUGCAGACGGAAGUCCAGCGGAGGC
AAGGGCGGCAGCUACAGCCAGGCCGCCAGCUCUGAUAGCGCCCAGGGC
AGCGACGUGUCACUGACAGCC.

107. The method of any one of claims 91-106, wherein the 3′ untranslated region of the AES mRNA comprises the sequence
(SEQ ID NO: 33)
CUGGUACUGCAUGCACGCAAUGCUAGCUGCCCCUUUCCCGUCCUGGGU
ACCCCGAGUCUCCCCCGACCUCGGGUCCCAGGUAUGCUCCCACCUCCA
CCUGCCCCACUCACCACCUCUGCUAGUUCCAGACACCUCC.

108. The method of any one of claims 91-107, wherein the non-coding RNA of the mitochondrially encoded 12S RNA comprises the sequence
(SEQ ID NO: 35)
CAAGCACGCAGCAAUGCAGCUCAAAACGCUUAGCCUAGCCACACCCCC
ACGGGAAACAGCAGUGAUUAACCUUUAGCAAUAAACGAAAGUUUAACU
AAGCUAUACUAACCCCAGGGUUGGUCAAUUUCGUGCCAGCCACACCG.

109. The method of any one of claims 91-108, wherein the 3′ UTR comprises the sequence
(SEQ ID NO: 31)
CUCGAGCUGGUACUGCAUGCACGCAAUGCUAGCUGCCCCUUUCCCGUC
CUGGGUACCCCGAGUCUCCCCCGACCUCGGGUCCCAGGUAUGCUCCCA
CCUCCACCUGCCCCACUCACCACCUCUGCUAGUUCCAGACACCUCCCA
AGCACGCAGCAAUGCAGCUCAAAACGCUUAGCCUAGCCACACCCCCAC
GGGAAACAGCAGUGAUUAACCUUUAGCAAUAAACGAAAGUUUAACUAA
GCUAUACUAACCCCAGGGUUGGUCAAUUUCGUGCCAGCCACACCGAGA
CCUGGUCCAGAGUCGCUAGCCGCGUCGCU.

110. The method of any one of claims 91-109, wherein the poly(A) sequence comprises 120 adenine nucleotides.

111. The method of any one of claims 1-90, wherein the RNA vaccine comprises an RNA molecule comprising, in the 5′≥3′ direction:
the polynucleotide sequence
(SEQ ID NO: 19)
GGCGAACUAGUAUUCUUCUGGUCCCCACAGACUCAGAGAGAACCCGCC
ACCAUGAGAGUGAUGGCCCCCAGAACCCUGAUCCUGCUGCUGUCUGGC
GCCCUGGCCCUGACAGAGACAUGGGCCGGAAGC;
a polynucleotide sequence encoding the one or more neoepitopes resulting from cancer-specific somatic mutations present in the tumor specimen; and
the polynucleotide sequence
(SEQ ID NO: 20)
AUCGUGGGAAUUGUGGCAGGACUGGCAGUGCUGGCCGUGGUGGUGAUC
GGAGCCGUGGUGGCUACCGUGAUGUGCAGACGGAAGUCCAGCGGAGGC
AAGGGCGGCAGCUACAGCCAGGCCGCCAGCUCUGAUAGCGCCCAGGGC
AGCGACGUGUCACUGACAGCCUAGUAACUCGAGCUGGUACUGCAUGCA
CGCAAUGCUAGCUGCCCCUUUCCCGUCCUGGGUACCCCGAGUCUCCCC
CGACCUCGGGUCCCAGGUAUGCUCCCACCUCCACCUGCCCCACUCACC
ACCUCUGCUAGUUCCAGACACCUCCCAAGCACGCAGCAAUGCAGCUCA
AAACGCUUAGCCUAGCCACACCCCCACGGGAAACAGCAGUGAUUAACC
UUUAGCAAUAAACGAAAGUUUAACUAAGCUAUACUAACCCCAGGGUUG
GUCAAUUUCGUGCCAGCCACACCGAGACCUGGUCCAGAGUCGCUAGCC
GCGUCGCU.

112. The method of any one of claims 1-111, wherein the pancreatic cancer tumor is a resectable PDAC tumor, assessed by preoperative imaging in the patient with computed tomography (CT) scan with contrast or magnetic resonance imaging (MRI) prior to administration of the RNA vaccine, the PD-1 axis binding antagonist, and the chemotherapeutic treatment.

113. The method of any one of claims 1-112, wherein the pancreatic cancer tumor is a resectable PDAC tumor comprising one or more characteristics selected from the group consisting of:
clear fat plane around the celiac and superior mesenteric arteries;
patent superior mesenteric and portal veins;
no encasement of the superior mesenteric vein or portal veins;
no encasement of the superior mesenteric or hepatic arteries;
absence of metastatic disease; and
absence of extra-regional nodal disease.

114. The method of any one of claims 1-113, wherein the patient has a histologically confirmed diagnosis of PDAC prior to administration of the RNA vaccine, the PD-1 axis binding antagonist, and the chemotherapeutic treatment.

115. The method of any one of claims 1-114, wherein the patient has adenosquamous carcinoma of the pancreas prior to administration of the RNA vaccine, the PD-1 axis binding antagonist, and the chemotherapeutic treatment.

116. The method of any one of claims 1-115, wherein the pancreatic cancer tumor has tumor, lymph node, metastasis (TNM) pathological staging values of T1-T3, N0-N2, or MO prior to administration of the RNA vaccine, the PD-1 axis binding antagonist, and the chemotherapeutic treatment.

117. The method of any one of claims 1-116, wherein the pancreatic cancer tumor is a resectable PDAC tumor, and wherein:
the patient had no evidence of PDAC disease after resection of the PDAC tumor, and/or
the patient had a macroscopically complete resection of the PDAC tumor,
prior to administration of the RNA vaccine, the PD-1 axis binding antagonist, and the chemotherapeutic treatment, optionally wherein the patient had a RO or RI resection of the PDAC tumor.

118. The method of claim 117, wherein the patient had unequivocal absence of PDAC after resection of the PDAC tumor, optionally wherein the absence of PDAC is assessed by CT or MRI scans, one or more biochemical assays and/or clinical findings.

119. The method of any one of claims 1-118, wherein the pancreatic cancer tumor is a resectable PDAC tumor, and wherein following resection of the tumor, the patient did not have unresolved ≥Grade 3 postoperative complications prior to administration of the RNA vaccine, the PD-1 axis binding antagonist, and the chemotherapeutic treatment, optionally wherein the complications are assessed according to the Clavien-Dindo Classification of Surgical Complications.

120. The method of any one of claims 1-119, wherein the patient has a CA19-9 level of 180 U/mL or greater prior to administration of the RNA vaccine, the PD-1 axis binding antagonist, and the chemotherapeutic treatment.

121. The method of any one of claims 1-119, wherein the patient has a CA19-9 level of less than 180 U/mL prior to administration of the RNA vaccine, the PD-1 axis binding antagonist, and the chemotherapeutic treatment.

122. The method of any one of claims 1-121, wherein at least five neoepitopes resulting from cancer-specific somatic mutations are present in the tumor specimen obtained from the patient prior to administration of the RNA vaccine, the PD-1 axis binding antagonist, and the chemotherapeutic treatment.

123. The method of any one of claims 1-122, wherein the patient has an Eastern Cooperative Oncology Group (ECOG) Performance Status of 0 or 1 prior to administration of the RNA vaccine, the PD-1 axis binding antagonist, and the chemotherapeutic treatment.

124. The method of any one of claims 1-123, wherein the patient does not have intraductal papillary mucinous neoplasm-associated PDAC prior to administration of the RNA vaccine, the PD-1 axis binding antagonist, and the chemotherapeutic treatment.

125. The method of any one of claims 1-124, wherein the patient does not have a pancreatic endocrine tumor or acinar cell adenocarcinoma, pancreatic cystadenocarcinoma, or pancreatic malignant ampulloma prior to administration of the RNA vaccine, the PD-1 axis binding antagonist, and the chemotherapeutic treatment.

126. The method of any one of claims 1-125, wherein the patient has not received an adjuvant, neoadjuvant, or induction treatment for pancreatic cancer, or a systemic anti-cancer treatment for pancreatic cancer, prior to administration of the RNA vaccine, the PD-1 axis binding antagonist, and the chemotherapeutic treatment; optionally wherein the pancreatic cancer is PDAC.

127. The method of any one of claims 1-126, wherein the patient has not had a cytotoxic chemotherapy, immunotherapy, investigational therapy, or radiation therapy prior to administration of the RNA vaccine, the PD-1 axis binding antagonist, and the chemotherapeutic treatment.

128. The method of any one of claims 1-127, wherein the patient has a spleen prior to administration of the RNA vaccine, the PD-1 axis binding antagonist, and the chemotherapeutic treatment.

129. The method of any one of claims 1-128, wherein the patient has not had loss of spleen due to splenectomy, splenic injury/infarction, or functional asplenia prior to administration of the RNA vaccine, the PD-1 axis binding antagonist, and the chemotherapeutic treatment.

130. The method of any one of claims 1-129, wherein the patient has not had a distal pancreatectomy with splenectomy prior to administration of the RNA vaccine, the PD-1 axis binding antagonist, and the chemotherapeutic treatment.

131. The method of any one of claims 1-130, wherein the patient does not have preexisting neuropathy prior to administration of the RNA vaccine, the PD-1 axis binding antagonist, and the chemotherapeutic treatment.

132. The method of any one of claims 1-131, wherein the patient does not have an aUGT1A1 genotype associated with poor metabolizer phenotype prior to administration of the RNA vaccine, the PD-1 axis binding antagonist, and the chemotherapeutic treatment.

133. The method of any one of claims 1-132, wherein the patient does not have an autoimmune disease, immune deficiency, or primary immunodeficiency prior to administration of the RNA vaccine, the PD-1 axis binding antagonist, and the chemotherapeutic treatment.

134. The method of any one of claims 1-133, wherein the patient has not been treated with:
monoamine oxidase inhibitors (MAOIs) within 3 weeks,
a systemic immunostimulatory agent within 4 weeks or 5 drug-elimination half-lives, whichever is longer, or
a systemic immunosuppressive medication within 2 weeks,
prior to administration of the RNA vaccine, the PD-1 axis binding antagonist, and the chemotherapeutic treatment.

135. The method of any one of claims 1-134, wherein the patient has not had an allogeneic stem cell or solid organ transplantation prior to administration of the RNA vaccine, the PD-1 axis binding antagonist, and the chemotherapeutic treatment.

136. The method of any one of claims 1-135, further comprising assessing disease-free survival (DFS) of the patient after treatment with the RNA vaccine, the PD-1 axis binding antagonist, and the chemotherapeutic treatment.

137. The method of claim 136, wherein administration of the RNA vaccine, the PD-1 axis binding antagonist, and the chemotherapeutic treatment results in an improvement in DFS of the patient as compared to DFS of a corresponding patient not administered the RNA vaccine, the PD-1 axis binding antagonist, and the chemotherapeutic treatment.

138. The method of any one of claims 1-137, further comprising assessing overall survival (OS) of the patient after treatment with the RNA vaccine, the PD-1 axis binding antagonist, and the chemotherapeutic treatment.

139. The method of claim 138, wherein administration of the RNA vaccine, the PD-1 axis binding antagonist, and the chemotherapeutic treatment results in an improvement in OS of the patient as compared to OS of a corresponding patient not administered the RNA vaccine, the PD-1 axis binding antagonist, and the chemotherapeutic treatment.

140. The method of any one of claims 1-139, further comprising performing one or more clinical assessments of the patient before, during and/or after treatment with the RNA vaccine, the PD-1 axis binding antagonist, and the chemotherapeutic treatment, wherein the one or more clinical assessments are selected from the group consisting of European Organisation for Research and Treatment of Cancer QLQ-C30 Questionnaire (EORTC QLQ C30), European Organisation for Research and Treatment of Cancer QLQ-PAN26 Questionnaire (EORTC QLQ PAN26), National Cancer Institute's Patient-Reported Outcomes Common Terminology Criteria for Adverse Events (PRO CTCAE), and European Organisation for Research and Treatment of Cancer Item Library 46 Questionnaire (EORTC IL46).

141. The method of claim 140, wherein administration of the RNA vaccine, the PD-1 axis binding antagonist, and the chemotherapeutic treatment results in an improvement in the one or more clinical assessments as compared to the one or more clinical assessments in the patient prior to administration of the RNA vaccine, the PD-1 axis binding antagonist, and the chemotherapeutic treatment, and/or as compared to the one or more clinical assessments in a corresponding patient not administered the RNA vaccine, the PD-1 axis binding antagonist, and the chemotherapeutic treatment.

142. The method of any one of claims 1-141, further comprising assessing antigen- and/or tumor-specific T-cell responses in the patient before, during and/or after treatment with the RNA vaccine, the PD-1 axis binding antagonist, and the chemotherapeutic treatment.

143. The method of claim 142, wherein administration of the RNA vaccine, the PD-1 axis binding antagonist, and the chemotherapeutic treatment results in an improvement in antigen- and/or tumor-specific T-cell responses in the patient as compared to prior to administration of the RNA vaccine, the PD-1 axis binding antagonist, and the chemotherapeutic treatment, and/or as compared to a corresponding patient not administered the RNA vaccine, the PD-1 axis binding antagonist, and the chemotherapeutic treatment.

144. The method of any one of claims 137, 139, 141, and 143, wherein the corresponding patient is a patient with a corresponding pancreatic cancer tumor, optionally wherein the pancreatic cancer tumor is a PDAC tumor and the corresponding patient has a PDAC tumor.

145. The method of any one of claims 137, 139, 141, and 143-144, wherein the corresponding patient was treated with a standard of care treatment for pancreatic cancer, PDAC, or resectable or resected PDAC.

146. The method of claim 145, wherein the standard of care treatment comprises a gemcitabine combination therapy or an mFOLFIRINOX chemotherapy.

147. The method of any one of claims 137, 139, 141, and 143-144, wherein the corresponding patient was treated with a control treatment comprising an mFOLFIRINOX chemotherapy.

148. The method of claim 146 or claim 147, wherein the mFOLFIRINOX chemotherapy comprises oxaliplatin at dose of about 85 mg/m², leucovorin at dose of about 400 mg/m², irinotecan at dose of about 150 mg/m², and 5-fluorouracil at dose of about 2400 mg/m², administered intravenously in 14-day cycles, on day 1 of each cycle for a total of up to 12 cycles.

149. The method of any one of claims 1-148, wherein the RNA vaccine dose is administered to the patient in two equal half-doses.

150. The method of claim 149, wherein the two equal half-doses are administered sequentially, optionally with an observation period between the administered equal half-doses.

151. The method of claim 89, wherein the dose of about 25 μg is split into two equal half-doses of about 12.5 μg, each administered over 1 minute, optionally with a 5-minute observation period between the administered equal half-doses.

152. An individualized RNA vaccine for use in a method for treating a pancreatic cancer tumor in a human patient in need thereof, wherein the RNA vaccine is to be administered in combination with a PD-1 axis binding antagonist and a chemotherapeutic treatment according to the method of any one of claims 1-151,
wherein the RNA vaccine comprises one or more polynucleotides encoding one or more neoepitopes resulting from cancer-specific somatic mutations present in a pancreatic cancer tumor specimen obtained from the patient.

153. A PD-1 axis binding antagonist for use in a method for treating a pancreatic cancer tumor in a human patient in need thereof, wherein the PD-1 axis binding antagonist is to be administered in combination with an individualized RNA vaccine and a chemotherapeutic treatment according to the method of any one of claims 1-151,
wherein the RNA vaccine comprises one or more polynucleotides encoding one or more neoepitopes resulting from cancer-specific somatic mutations present in a pancreatic cancer tumor specimen obtained from the patient.

154. Use of an individualized RNA vaccine in the manufacture of a medicament for treating a pancreatic cancer tumor in a human patient in need thereof, wherein the RNA vaccine is to be administered in combination with a PD-1 axis binding antagonist and a chemotherapeutic treatment according to the method of any one of claims 1-151, and
wherein the RNA vaccine comprises one or more polynucleotides encoding one or more neoepitopes resulting from cancer-specific somatic mutations present in a pancreatic cancer tumor specimen obtained from the patient.

155. Use of a PD-1 axis binding antagonist in the manufacture of a medicament for treating a pancreatic cancer tumor in a human patient in need thereof, wherein the PD-1 axis binding antagonist is to be administered in combination with an individualized RNA vaccine and a chemotherapeutic treatment according to the method of any one of claims 1-151, and
wherein the RNA vaccine comprises one or more polynucleotides encoding one or more neoepitopes resulting from cancer-specific somatic mutations present in a pancreatic cancer tumor specimen obtained from the patient.

156. A kit comprising an individualized RNA vaccine, for use in a method for treating a pancreatic cancer tumor in a human patient in need thereof, wherein the RNA vaccine is to be administered in combination with a PD-1 axis binding antagonist and a chemotherapeutic treatment according to the method of any one of claims 1-151,
wherein the RNA vaccine comprises one or more polynucleotides encoding one or more neoepitopes resulting from cancer-specific somatic mutations present in a pancreatic cancer tumor specimen obtained from the patient.

157. A kit comprising a PD-1 axis binding antagonist for use in a method for treating a pancreatic cancer tumor in a human patient in need thereof, wherein the PD-1 axis binding antagonist is to be administered in combination with an individualized RNA vaccine and a chemotherapeutic treatment according to the method of any one of claims 1-151,
wherein the RNA vaccine comprises one or more polynucleotides encoding one or more neoepitopes resulting from cancer-specific somatic mutations present in a pancreatic cancer tumor specimen obtained from the patient.

158. A method of selecting a human patient having a cancer tumor as likely to respond to a therapy comprising an individualized RNA vaccine, the method comprising:
a) measuring the number and/or frequency of de novo significantly expanded (SE) TCR clones in a sample from the patient by T cell receptor sequencing;
b) comparing the number and/or frequency of de novo SE TCR clones measured in a) to a reference number and/or frequency; and
c) selecting the patient as more likely to respond to the therapy comprising the individualized RNA vaccine when the number and/or frequency of de novo SE TCR clones in the sample from the patient is above the reference number and/or frequency;
wherein the individualized RNA vaccine comprises one or more polynucleotides encoding one or more neoepitopes resulting from cancer-specific somatic mutations present in a cancer tumor specimen obtained from the patient, and
wherein a number and/or frequency of de novo SE TCR clones above the reference number and/or frequency indicates that the patient is more likely to respond to the therapy comprising the individualized RNA vaccine.

159. A method of selecting a human patient having a cancer tumor as likely to respond to a therapy comprising an individualized RNA vaccine, the method comprising:
a) comparing the number and/or frequency of de novo significantly expanded (SE) TCR clones in a sample from the patient to a reference number and/or frequency; and
b) selecting the patient as more likely to respond to the therapy comprising the individualized RNA vaccine when the number and/or frequency of de novo SE TCR clones in the sample from the patient is above the reference number and/or frequency;
wherein the individualized RNA vaccine comprises one or more polynucleotides encoding one or more neoepitopes resulting from cancer-specific somatic mutations present in a cancer tumor specimen obtained from the patient,
wherein the number and/or frequency of de novo SE TCR clones is measured by T cell receptor sequencing, and
wherein a number and/or frequency of de novo SE TCR clones above the reference number and/or frequency indicates that the patient is more likely to respond to the therapy comprising the individualized RNA vaccine.

160. The method of claim 158 or claim 159, further comprising selecting the therapy comprising the individualized RNA vaccine or recommending the therapy comprising the individualized RNA vaccine.

161. A method of treating a human patient having a cancer tumor, the method comprising:
a) measuring the number and/or frequency of de novo significantly expanded (SE) TCR clones in a sample from the patient by T cell receptor sequencing;
b) comparing the number and/or frequency of de novo SE TCR clones measured in a) to a reference number and/or frequency; and
c) selecting the patient as more likely to respond to the therapy comprising the individualized RNA vaccine when the number and/or frequency of de novo SE TCR clones in the sample from the patient is above the reference number and/or frequency, thereby treating the cancer tumor;
wherein the individualized RNA vaccine comprises one or more polynucleotides encoding one or more neoepitopes resulting from cancer-specific somatic mutations present in a cancer tumor specimen obtained from the patient, and
wherein a number and/or frequency of de novo SE TCR clones above the reference number and/or frequency indicates that the patient is more likely to respond to the therapy comprising the individualized RNA vaccine.

162. A method of treating a human patient having a cancer tumor, the method comprising:
a) comparing the number and/or frequency of de novo significantly expanded (SE) TCR clones in a sample from the patient to a reference number and/or frequency; and
b) selecting the patient as more likely to respond to the therapy comprising the individualized RNA vaccine when the number and/or frequency of de novo SE TCR clones in the sample from the patient is above the reference number and/or frequency, thereby treating the cancer tumor;
wherein the individualized RNA vaccine comprises one or more polynucleotides encoding one or more neoepitopes resulting from cancer-specific somatic mutations present in a cancer tumor specimen obtained from the patient,
wherein the number and/or frequency of de novo SE TCR clones is measured by T cell receptor sequencing, and
wherein a number and/or frequency of de novo SE TCR clones above the reference number and/or frequency indicates that the patient is more likely to respond to the therapy comprising the individualized RNA vaccine.

163. The method of claim 161 or claim 162, further comprising administering the therapy comprising the individualized RNA vaccine to the patient when the number and/or frequency of de novo SE TCR clones in the sample from the patient is above the reference number and/or frequency, thereby treating the cancer tumor.

164. The method of claim 163, further comprising selecting the therapy comprising the individualized RNA vaccine when the number and/or frequency of de novo SE TCR clones in the sample from the patient is above the reference number and/or frequency, thereby treating the cancer tumor.

165. The method of any one of claims 158-164, wherein the number and/or frequency is measured after six doses of the individualized cancer vaccine.

166. The method of any one of claims 158-165, wherein the reference number is six de novo SE TCR clones.

167. The method of any one of claims 158-165, wherein the reference frequency is 104 de novo SE TCR clones.

168. The method of any one of claims 158-167, wherein the cancer tumor is a pancreatic cancer tumor.

169. The method of any one of claims 158-168, wherein the cancer tumor is a pancreatic ductal adenocarcinoma (PDAC) tumor.

170. The method of any one of claims 158-169, wherein the therapy comprising the individualized RNA vaccine further comprises a PD-1 axis binding antagonist.

171. The method of claim 170, wherein the therapy further comprises a chemotherapeutic treatment and wherein the RNA vaccine, the PD-1 axis binding antagonist, and the chemotherapeutic treatment are administered to the patient during a priming phase, a chemotherapy phase after the priming phase, and a boost phase after the chemotherapy phase, wherein:
(i) the priming phase comprises administering to the patient at least one dose of the RNA vaccine and at least one dose of the PD-1 axis binding antagonist,
(ii) the chemotherapy phase comprises administering to the patient the chemotherapeutic treatment, and
(iii) the boost phase comprises administering to the patient at least one dose of the RNA vaccine and at least one dose of the PD-1 axis binding antagonist.

172. The method of claim 170 or 171, wherein the PD-1 axis binding antagonist is atezolizumab.

173. The method of claim 171 or 172, wherein the chemotherapeutic treatment is a FOLFIRINOX treatment or an mFOLFIRINOX treatment.

174. The method of any one of claims 1-151, wherein, prior to the administering step, the patient is selected by a method comprising:
a) measuring the number and/or frequency of de novo significantly expanded (SE) TCR clones in a sample from the patient by T cell receptor sequencing;
b) comparing the number and/or frequency of de novo SE TCR clones measured in a) to a reference number and/or frequency; and
c) selecting the patient as more likely to respond to the therapy comprising the individualized RNA vaccine when the number and/or frequency of de novo SE TCR clones in the sample from the patient is above the reference number and/or frequency;
wherein the individualized RNA vaccine comprises one or more polynucleotides encoding one or more neoepitopes resulting from cancer-specific somatic mutations present in a cancer tumor specimen obtained from the patient, and
wherein a number and/or frequency of de novo SE TCR clones above the reference number and/or frequency indicates that the patient is more likely to respond to the therapy comprising the individualized RNA vaccine.

175. In vitro use of number and/or frequency of de novo significantly expanded (SE) TCR clones for selecting a patient having a cancer tumor more likely to respond to a therapy comprising an individualized RNA vaccine, wherein a number and/or frequency of de novo SE TCR clones in the sample from the patient above a reference number and/or frequency selects that the patient is more likely to respond to the therapy comprising the individualized RNA vaccine.

176. Use of a number and/or frequency of de novo significantly expanded (SE) TCR clones for the manufacture of a diagnostic for assessing the likelihood of a response of a patient having a cancer tumor to a therapy comprising an individualized RNA vaccine.
